(19) United States
(12) Patent Application Publication
(10) Pub. No.: US 2025/0212918 A1
(43) Pub. Date: Jul. 3, 2025

(54) METHOD FOR ARABINOXYLAN OLIGOSACCARIDE EXTRACTION FROM CORN MATERIAL

(71) Applicant: TATE & LYLE SOLUTIONS USA LLC, Hoffman Estates, IL (US)

(72) Inventors: Xin Nie, Hoffman Estates, IL (US); James Gaddy, Hoffman Estates, IL (US); Juhi Tomar, Hoffman Estates, IL (US); Zheng You, Hoffman Estates, IL (US)

(21) Appl. No.: 18/852,045

(22) PCT Filed: Mar. 30, 2023
(86) PCT No.: PCT/US2023/016895

§ 371 Date: Sep. 27, 2024

Related U.S. Application Data
(60) Provisional application No. 63/325,971, filed on Mar. 31, 2022.
(60) Provisional application No. 63/405,628, filed on Sep. 12, 2022.

(51) Int. Cl. A23L 7/104; A23L 5/20; A23L 33/21; C12N 9/26; C12N 9/42

(52) U.S. Cl. CPC A23L 7/107; A23L 5/25; A23L 33/21; C12N 9/2414; C12N 9/2437; C12Y 302/01001; C12Y 302/01004; C12Y 302/01008

(57) ABSTRACT

The present disclosure related generally to a method of providing an arabinoxylan product from a corn material such as a corn fiber. The method includes de-starching the corn material to provide a de-starched corn material; maintaining an aqueous mixture of the destarched corn material at an extraction pH in the range of 5-14 for a time and at a temperature effective to extract arabinoxylan from the de-starched corn material into an aqueous phase of the aqueous mixture and to provide a corn residue; separating the aqueous phase containing the extracted arabinoxylan from the corn residue; and providing the arabinoxylan product from the aqueous phase.

### FIELD OF THE DISCLOSURE

The present disclosure relates generally to soluble fiber extraction and isolation from corn-based materials, such as arabinoxylan oligosaccharide extraction and isolation from corn fiber.

### TECHNICAL BACKGROUND

Arabinoxylan fibers are natural extracts of plants that can provide advantages to prebiotic health by increasing the absorption of calcium and magnesium, reducing cholesterol absorption, lowering plasma cholesterol, decreasing cholesterol accumulation in the liver, and contributing to desirable bifidogenic effects. The prebiotic benefits of arabinoxylan products on gut microbiota adjustment and chronic disease are attractive in today's health and wellness market.

Arabinoxylan is a polysaccharide having a backbone repeat unit of [β-(1,4)-D-xylopyranose]. The backbone is substituted with, inter alia, arabinose side chains, often with a portion of the arabinose side chains being esterified with phenolic compounds, in particular phenolic acids such as coumaric acid and ferulic acid (e.g., one or two phenolic acid moieties may be substituted on a single xylose repeat unit of the backbone). Partial hydrolysis of polymeric arabinoxylan can yield arabinoxylan oligosaccharides, which are sometimes referred to by the acronym “AXOS.” As used herein, “arabinoxylan” refers to arabinoxylan in polymeric or oligomeric form.

Arabinoxylan is known to be present in corn materials like so-called “corn fiber,” e.g., the fibrous residue resulting as a product stream in the wet milling of corn. The arabinoxylan polysaccharides in com fiber are tough, gummy, high molecular weight substances that are covalently crosslinked (e.g., through ferulic acid ester substituents) both to themselves and to lignin structures in the com fiber. Extracting arabinoxylan substances in a soluble form from the corn fiber generally involves a cleavage of the covalent bonds involved in such crosslinking. However, extraction of arabinoxylan from com fiber still faces challenges in providing a high quality product in an effective and efficient matter. There remains a need in the art to improve extraction methods of arabinoxylan. Here, the present inventors have found a method of providing an arabinoxylan product with high quality and yield.

### SUMMARY

In one aspect, the present disclosure provides a method of providing an arabinoxylan product from corn fiber, the method comprising:
providing a corn material (e.g., a corn fiber);
de-starching the com material to provide a de-starched com material (e.g., a destarched corn fiber);
maintaining an aqueous mixture of the de-starched corn material at an extraction pH in the range of 5-14 for a time and at a temperature effective to extract arabinoxylan from the de-starched com material into an aqueous phase of the aqueous mixture and to provide a corn residue (e.g., a corn fiber residue);
separating the aqueous phase containing the extracted arabinoxylan from the corn residue; and
providing the arabinoxylan product from the aqueous phase.

Another aspect of the present disclosure provides an arabinoxylan product prepared by the methods as described herein. In one aspect of the present disclosure, the arabinoxylan product has:
a dietary fiber content in the range of 60-95% by weight on a dry solids basis;
a digestible carbohydrate content of less than 20% by weight on a dry solids basis;
a protein content of less than 15% by weight on a dry solids basis; and
a fat of less than 10% by weight on a dry solids basis.

Another aspect of the present disclosure provides a method for making a food product, the method comprising providing an arabinoxylan product as described herein or an arabinoxylan product prepared by the method as described herein and combining it with one or more other food ingredients. Another aspect of the present disclosure provides a food product made by the method as described herein. Another aspect of the present disclosure provides a food product comprising an arabinoxylan product as described herein or an arabinoxylan product prepared by the method as described herein.

### BRIEF DESCRIPTION OF FIGURES

The accompanying drawings are included to provide a further understanding of the methods of the disclosure, and are incorporated in and constitute a part of this specification. The drawings are not necessarily to scale, and sizes of various elements may be distorted for clarity. The drawings illustrate one or more embodiment(s) of the disclosure, and together with the description serve to explain the principles and operation of the disclosure.

FIG. 1 is a schematic of the extraction process as described herein under neutral conditions.

FIG. 2 is a schematic of a comparative extraction process under basic conditions.

FIG. 3 is a schematic of a comparative extraction process under acidic conditions.

FIG. 4 is a graph of the extraction product content extracted under neutral, basic and acidic conditions.

FIG. 5 is a graph of the extraction product content from a dent corn fiber feed and a waxy corn fiber feed, treated with different de-starching enzymes.

FIG. 6 is a graph of the amount of starch in corn fiber and de-starched products, as grams starch per 200 grams corn fiber sample.

FIG. 7 is a graph of the extraction product content extracted under neutral conditions for 1.5 and 3 hours.

FIG. 8 is a chromatogram of the extraction products obtained from extracted under neutral conditions at 1.5 and 3 hours.

FIG. 9 is a graph of the starch content of de-starched products, treated with destarching enzymes.

FIG. 10 is a bar graph of the starch content of de-starched products, treated with de-starching enzymes and washing.

FIG. 11 is a bar graph of the starch content of de-starched products, treated with de-starching enzymes at different reaction time1.

FIG. 12 is a graph of the starch content of de-starched products, treated with destarching enzymes.

FIG. 13A is a chromatogram of the extraction products obtained before nano-filtration.

FIG. 13B is a chromatogram of the extraction products obtained after nano-filtration.

FIG. 14 is a graph of shear rate vs. viscosity for different arabinoxylan products treated with enzymes.

FIG. 15 is a schematic of the extraction and purification process as described herein.

FIG. 16A is a picture of extracted arabinoxylan prepared by the process as described here.

FIG. 16B is a picture of extracted arabinoxylan prepared by the process as described here.

FIG. 17A is a viscosity curve of the arabinoxylan product prepared by the processes as described herein at different pHs and solid content

FIG. 17B is a viscosity curve of the arabinoxylan product prepared by the processes (C-AX) as described herein in comparison with wheat arabinoxylan medium viscosity (WM) and wheat arabinoxylan low viscosity (WL) Both WM and WL are obtained from Megazyme. (Product codes: P-WAXYM, PWAXYL)

FIG. 18A is a picture of the arabinoxylan product dispersed in solution.

FIG. 18B is a picture of the arabinoxylan product dispersed in solution.

FIG. 19 is a schematic of the extraction and purification process for hydrolyzed arabinoxylan as described herein.

FIG. 20 is a picture of hydrolyzed arabinoxylan prepared by the process as described here.

FIG. 21A is a viscosity curve of the arabinoxylan product without enzyme hydrolysis prepared by the processes as described herein.

FIG. 21B is a viscosity curve of the arabinoxylan product with enzyme hydrolysis prepared by the processes as described herein.

FIG. 21C is a viscosity curve of the arabinoxylan product with enzyme hydrolysis prepared by the processes as described herein.

FIG. 21D is a viscosity curve of a comparative soluble arabinoxylan product.

FIG. 21E is a plot of the viscosity versus temperature of the arabinoxylan product prepared by the processes as described herein.

FIG. 22 is a picture of the arabinoxylan product with enzyme hydrolysis dispersed in solution.

FIG. 23A is a graph of the change in mass versus relative humidity of the arabinoxylan product prepared by the processes as described herein.

FIG. 23B is a graph of the change in mass versus time of the arabinoxylan product prepared by the processes as described herein.

FIG. 24A is a chromatograph of an arabinoxylan product as described herein treated with an endoxylanase.

FIG. 24B is a chromatograph of an arabinoxylan product as described herein treated with an endoxylanase.

FIG. 25A is a chromatograph of a comparative arabinoxylan product treated with an endoxylanase.

FIG. 25B is a chromatograph of a comparative arabinoxylan product treated with an endoxylanase.

FIG. 25C is a chromatograph of a comparative arabinoxylan product treated with an endoxylanase.

FIG. 26 is a chromatograph of an arabinoxylan product as described herein treated with an endoxylanase.

FIG. 27A is a chromatograph of an arabinoxylan product as described herein before and after treatment with different enzymes. :XOS is a comparative xylo oligosaccharide product as a reference.

FIG. 27B is a chromatograph of a comparative arabinoxylan product treated with different enzymes.

FIG. 27C is a chromatograph of a comparative arabinoxylan product treated with different enzymes.

FIG. 28 is a GPC distribution plot of arabinoxylan product as described herein treated with different enzymes.

FIG. 29 is a time course of AX crude extract hydrolysis by FRONTIA® Fiber Wash 2.0.

FIG. 30 is a time course of AX spray-dried hydrolysis by FRONTIA® Fiber Wash 2.0.

FIG. 31A is a time course of purified AX treated by FRONTIA® Fiber Wash 2.0.

FIG. 31B is a plot of viscosity of AX samples after treatment by FRONTIA® Fiber Wash 2.0 for different time period at different shear rates.

FIG. 32 is a photograph of the arabinoxylan product in solution before and after treatment with FRONTIA® Fiber Wash 2.0 as described herein.

FIG. 33 Time course for molecular weight reduction using FRONTIA® Fiber Wash and FRONTIA® Fiber Wash 2.0.

FIG. 34 is a photograph of emulsions prepared with the arabinoxylan products as described herein.

FIG. 35 is a photograph of emulsions prepared with the arabinoxylan products as described herein.

### DETAILED DESCRIPTION

The present inventors have noted that current methods used in extracting arabinoxylan oligosaccharides from corn fiber, such as under highly acidic conditions, do not provide the desired high quality arabinoxylan product necessary for use in food products. Using highly acidic conditions for extracting arabinoxylan oligomers from corn fiber can significantly degrade the arabinoxylan oligosaccharides (e.g., generates relatively large amounts of mono-and disaccharide) and therefore require close control of the process parameters (e.g., amounts of acid and com fiber, temperature). The residual acid in the extract also must be neutralized, generating salts which typically must be removed to make the extract suitable for use as a food grade ingredient.

Accordingly, the present inventors have developed a method for extraction of arabinoxylan from corn materials, such as corn fiber under conditions that overcome the shortcomings associated with acidic extraction conditions.

As described above, the present disclosure provides a method of providing an arabinoxylan product from a corn material such as a corn fiber, the method includes: providing a com material (e.g., a corn fiber); de-starching the corn material to provide a de-starched corn material (e.g., a de-starched corn fiber); maintaining an aqueous mixture of the de-starched corn material at an extraction pH in the range of 5-14 for a time and at a temperature effective to extract arabinoxylan from the de-starched corn material into an aqueous phase of the aqueous mixture to provide a corn residue (e.g., a corn fiber residue); separating the aqueous phase containing the extracted arabinoxylan from the corn residue; and providing the arabinoxylan product from the aqueous phase.

A variety of com materials include arabinoxylan that can be extracted using the processes described herein. While the present disclosure focuses specifically on one such corn material, corn fiber, the person of ordinary skill in the art will adapt the methods of the disclosure for use with other corn materials. Accordingly, the present disclosure in all embodiments and aspects contemplates the use of corn materials other than corn fiber, and the description herein with respect to corn fiber is understood to be generalizable with respect to other corn materials.

The source of the corn fiber is not particularly limited and can be selected from any source known to those skilled in the art. For example, in some embodiments of the disclosure as described herein, the corn fiber is a by-product of corn wet-milling. In particular embodiments, the corn fiber is corn pericarp.

In various embodiments of the present disclosure as otherwise described herein, especially as a by-product of corn wet-milling, the corn fiber is provided as an aqueous mixture having a degree of solids in the range of 5-40%. For example, in various embodiments as otherwise described herein, the corn fiber is provided as an aqueous mixture having a degree of solids in the range of 5-35%, or 5-30%, or 10-40%, or 10-35%, or 10-30%, or 15-40%, or 15-35%, or 15-30%, or 20-40%, or 20-35%, or 20-30%, or 25-40%, or 25-35%, or 25-30%. The material can be, e.g., diluted or concentrated as appropriate for further processing as described herein.

As described above, the method as described herein includes de-starching the corn material (e.g., com fiber) to provide a de-starched corn material (e.g., de-starched corn fiber). As will be understood by the person of ordinary skill in the art, there are many methods to de-starch corn materials such as corn fiber. While the de-starching method chosen here is not particularly limited, the present inventors have identified particularly suitable methods, such as washing with warm aqueous media and treatment with enzymes.

Accordingly, in various embodiments as otherwise described herein, de-starching the corn material (e.g., corn fiber) includes washing the com material with a washing aqueous medium having a temperature up to 80° C., e.g., up to 75° C. or up to 70° C. The aqueous washing medium desirably has a pH in the range of 5-9. Water, e.g., purified or from a municipal source, can advantageously be used as the aqueous washing medium.

The person of ordinary skill in the art can select washing conditions to provide the desired degree of de-starching of the corn material. For example, in various embodiments of the present disclosure, the washing aqueous medium has a temperature in the range of 20-80° C., e.g., or 20-75° C., or 20-70° C., or 30-80° C., or 30-75° C., or 30-70° C., or 40-80° C., or 40-75° C., or 40-70° C., or 50-80° C., or 50-75° C. or 50-70° C.

In various embodiments of the present disclosure, the mass of the washing aqueous medium is at least 5 times the mass on a dry solids basis of the corn fiber. For example, the mass of the washing aqueous medium may be at least 10 times or at least 20 times the mass on a dry solids basis of the corn fiber. When de-starching, the total amount of washing aqueous medium may be used in one wash cycle or in multiple washing cycles. For example, de-starching the corn fiber may include contacting the total mass of the washing aqueous medium with the corn fiber and separating the de-starched corn fiber from the used washing aqueous medium. In other examples, de-starching the corn fiber may include contacting an amount of the total mass of the washing aqueous medium with the corn fiber, separating the de-starched corn fiber from the used washing aqueous medium, and repeating the contacting and separating steps until the total mass of the washing aqueous medium is used. Similarly, the corn material may be continuously washed with the washing aqueous medium, with continuous removal of used washing aqueous medium.

In various embodiments of the disclosure, the washing is performed for a time that is no more than an hour, e.g., no more than 45 minutes, or no more than 30 minutes. As noted herein, contact with aqueous media at relatively neutral pH can, over time and at a higher temperature (e.g., greater than 80° C.), causes extraction of arabinoxylan from the corn material; the person of ordinary skill in the art can, based on the present disclosure, select washing conditions and times that provide a desired degree of de-starching without an undue amount of extraction of arabinoxylan into the washing aqueous medium.

In various embodiments of the present disclosure as described herein, de-starching the corn fiber comprises treating the corn fiber with one or more enzymes in an enzyme treatment aqueous medium. In some embodiments of the present disclosure as described herein, the one or more enzymes include one or more amylases, one or more glucoamylases, one or more xylanases, one or more arabinofuranosidases, and/or one or more cellulases. For example, in various embodiments, the one or more enzymes includes a mixture of xylanases and arabinofuranosidases, such as the FRONTIA® Fiber wash product available from Novozymes A/S. In other examples, the one or more enzymes include alpha-amylases, such as the BAN 480 LS enzyme, from Novozymes. In various embodiments, treating the corn fiber with one or more enzymes in an enzyme treatment aqueous medium may include one or more treatment steps. For example, in some embodiments, treating the corn fiber with one or more enzymes may including treating the corn fiber with a first enzyme treatment aqueous medium and then a second enzyme treatment aqueous medium. The first and second enzyme treatment aqueous medium may include the same or different enzymes. For example, in some embodiments, the first enzyme aqueous treatment medium Includes a mixture of xylanases and arabinofuranosidases and the second enzyme aqueous treatment medium includes alpha-amylase.

The present inventors have noted that the form of bound starch in corn materials like com fiber is largely granular starch, and that the granular starch is a desirable product stream. For example, enzymes like xylanases, arabinofuranosidases, and cellulases can provide a granular starch product stream while de-starching the corn fiber, as they primarily break bonds other than the alpha-1,4-and alpha-1,6-glycosidic bonds of the starch itself. For example, xylanases operate primarily on xylans like arabinoxylan, and cellulases operate primarily on cellulose. Breaking these bonds can free the starch granules from the mass of corn material without degrading the starch granules themselves. The resulting starch product stream can be purified and provided as a native corn starch, or can be modified using any desirable modifications. Normally, using amylases and glucoamylases in the de-starching can degrade the granular starch. However, the present inventors note that in practice, the amylases and glucoamylases do not influence recovery of the granular starch, if used in the right order, but can improve the quality of the fiber recovered.

The person of ordinary skill in the art will select suitable conditions depending on the particular enzymes used. For example, in various embodiments as otherwise described herein, the one or more enzymes are present in the enzyme treatment aqueous medium in a total amount of 0.02 to 3 wt. % based on the total weight of dried solids. In some embodiments, the one or more enzymes are present in the enzyme treatment aqueous medium in a total amount of 0.05 to 3 wt. %, or 0.1 to 3 wt. %, or 0.5 to 3 wt. %, or 1 to 3 wt. %, or 0.02 to 2 wt. %, or 0.05 to 2 wt. %, or 0.1 to 2 wt. %, or 0.5 to 2 wt. %, or 1 to 2 wt. %, or 0.02 to 1 wt. %, or 0.05 to 1 wt. %, or 0.1 to 1 wt. %, or 0.5 to 1 wt. %, based on the total weight of dietary starch. As will be understood by the person of ordinary skill in the art, the enzyme treatment aqueous medium can also include further components to provide a desired degree of enzyme stability and efficacy. For example, inorganic salts, inorganic acids and/or bases, and organic acids and/or bases, may be present in the enzyme treatment aqueous medium. In various embodiments of the present disclosure, and depending on the particular enzyme(s) used, treating the corn material with the one or more enzymes in an enzyme treatment aqueous medium is conducted at a pH in the range of 3 to 9. For example, in various embodiments the pH is in the range of 3 to 8, or 3 to 7, or 4 to 9, or 4 to 8, or 4 to 7. In some embodiments of the present disclosure as described herein, treating the corn fiber with one or more enzymes in an enzyme treatment aqueous medium is conducted for a time and at a temperature sufficient to de-starch the corn fiber. For example, in some embodiments of the present disclosure, treating the corn fiber with one or more enzymes in an enzyme treatment aqueous medium is conducted for a time of at least 30 minutes (e.g., at least 1 hour, at least 1.5 hours, at least 2 hours, at least 3 hours, or at least 3.5 hours). For example, in various embodiments, treating the corn fiber with one or more enzymes in an enzyme treatment aqueous medium is conducted for a time in the range of 1-6 hours, or 1-5 hours, or 1-4 hours, or 2-6 hours, or 2-5 hours, or 2-4 hours, or 3-6 hours, or 3-5 hour, or 3-4 hours. In some embodiments of the present disclosure, treating the corn fiber with one or more enzymes in an enzyme treatment aqueous medium is conducted for a temperature in the range of 25° C. to 70° C. For example, in various embodiments the temperature is in the range of 25° C. to 60° C., or 30° C. to 70° C., or 30° C. to 60° C. After de-starching with enzymes, further de-starching methods can be used. For example, in some embodiments, after treating the com material with one or more enzyme treatment aqueous medium, the de-starched com material is washed with an aqueous medium. The aqueous washing medium desirably has a pH in the range of 5-9. Water, e.g., purified or from a municipal source, can advantageously be used as the aqueous washing medium. In some embodiments of the present disclosure, the mass of the washing aqueous medium is in the range of 75%-200% of the mass of the corn material. For example, in various embodiments, the mass of the washing aqueous medium is at least 2 times, or at least 3 times the mass of the corn material. When de-starching, the total amount of washing aqueous medium may be used in one wash cycle or in multiple washing cycles. For example, de-starching the corn fiber may include contacting the total mass of the washing aqueous medium with the corn fiber and separating the de-starched corn fiber from the used washing aqueous medium. In other examples, de-starching the corn fiber may include contacting an amount of the total mass of the washing aqueous medium with the corn fiber, separating the de-starched corn fiber from the used washing aqueous medium, and repeating the contacting and separating steps until the total mass of the washing aqueous medium is used. Similarly, the corn material may be continuously washed with the washing aqueous medium, with continuous removal of used washing aqueous medium.

The de-starched corn material can be retained as a solid in the enzymatic de-starching reaction mixture; the person of ordinary skill in the art can use conventional methods (e.g., filtration, centrifugation, optionally followed by washing) to separate the solid de-starched corn material from the enzyme treatment aqueous medium containing the removed starch component. Depending on the destarching procedure, the person of ordinary skill in the art would be able to select an appropriate separation technique. For example, when the de-starching provides a particulate starch, filtration by size exclusion may be used to separate the solid de-starched corn material from the particulate starch component.

As noted above, the de-starching method is not particularly limited. But in particular embodiments of the present disclosure as described herein, the de-starching reduces a starch content of the corn material by at least 1%. For example, in various embodiments, the de-starching reduces a starch content of the corn material by at least 5 wt. %, or by at least 10 wt. %, or by at least 15 wt. %. In various embodiments as otherwise described herein, the de-starched corn material has no more than 20 wt. % starch on a dry solids basis. For example, in various embodiments, the de-starched corn material has no more than 15 wt. %, no more than 12 wt. %, no more than 10 wt. %, no more than 8 wt. %, or no more than 5 wt. % starch on a dry solids basis.

As described above, the method also includes maintaining an aqueous mixture of the de-starched corn fiber at an extraction pH in the range of 5-14. This step solubilizes arabinoxylan from the corn material into the aqueous medium, separating it from other components (e.g., solid lignin material) that remain as a solid corn residue. The present Inventors have determined that extraction at a pH in the range of 5-14 can provide a highly desirable combination of extraction efficiency and product characteristics. A variety of aqueous media can be used in this step; water (e.g., purified or from a municipal source) can be especially desirable.

The present inventors have noted that a variety of extraction pH values can be suitable. In various embodiments of the present disclosure as otherwise described herein, the extraction pH is in the range of 5 to 13, or 5 to 12.5, or 5 to 12, or 5 to 11.5, or 5 to 11, or 5 to 10, or 5 to 9, or 6 to 14, or 6 to 13, or 6 to 12.5, or 6 to 12, or 6 to 11.5, or 6 to 11, or 6 to 10, or 7 to 14, or 7 to 13, or 7 to 12.5, or 7 to 12, or 7 to 11.5, or 7 to 11, or 7 to 10, or 8 to 14, or 8 to 13, or 8 to 12.5, or 8 to 12, or 8 to 11.5, or 8 to 11, or 8 to 10. For example, the extraction pH is in the range of 6.2 to 14, or 6.2 to 13, or 6.2 to 12, or 6.2 to 11.5, or 6.2 to 11, or 6.2 to 10, or 6.2 to 9, or 7.2 to 14, or 7.2 to 13, or 7.2 to 12.5, or 7.2 to 12, or 7.2 to 11.5, or 7.2 to 11, or 7.2 to 10, or 8.2 to 14, or 8.2 to 13, or 8.2 to 12.5, or 8.2 to 12, or 8.2 to 11.5, or 8.2 to 11, or 8.2 to 10, or 6.5 to 14, or 6.5 to 13, or 6.5 to 12, 6.5 to 11.5, or 6.5 to 11, or 6.5 to 10, or 6.5 to 9, or 7.5 to 14, or 7.5 to 13, or 7.5 to 12.5, or 7.5 to 12, or 7.5 to 11.5, or 7.5 to 11, or 7.5 to 10, or 8.5 to 14, or 8.5 to 13, or 8.5 to 12.5, or 8.5 to 12, or 8.5 to 11.5, or 8.5 to 11, or 8.5 to 10. In some embodiments, the extraction pH is in the range of 9 to 14, e.g., in the range of 9 to 13, or 9 to 12.5, or 9 to 12, or 9 to 11.5, or 9 to 11, or 10 to 14, or 10 to 13, or 10 to 12.5, or 10 to 12, or 10 to 11.5, or 10.5 to 14, or 10.5 to 13, or 10.5 to 12.5, or 10.5 to 12, or 11 to 14, or 11 to 13, or 11 to 12.5, or 11.5 to 14, or 11.5 to 13, or 11.5 to 12.5.

In various embodiments of the present disclosure as otherwise described herein, the extraction pH is in the range of 5 to 9, for example, 5 to 8.5, or 5 to 8.2, or 5 to 8, or 5 to 7.5, or 5 to 7.2, or 5 to 7, or 6 to 9, or 6 to 8.5, or 6 to 8.2, or 6 to 8, or 6 to 7.5, or 6 to 7.2, or 6 to 7, or 6.2 to 9, or 6.2 to 8.5, or 6.2 to 8.2, or 6.2 to 8, or 6.2 to 7.5, or 6.2 to 7.2, or 6.2 to 7, or 6.5 to 9, or 6.2 to 8.5, or 6.2 to 8.2, or 6.5 to 8, or 6.5 to 7.5, or 6.5 to 7.2, or 6.5 to 7. In some embodiments of the present disclosure, one or more pH adjustments are performed on the aqueous mixture of de-starched corn fiber to provide the aqueous mixture the extraction pH. For example, the one or more pH adjustments may be performed by the addition of an aqueous medium (e.g., water) having a pH in the range of 5-9 (e.g., in the range of 5 to 8, or 5 to 7.5, or 6 to 9, or 6 to 8, or 6 to 7.5, or 6.2 to 9, or 6.2 to 8, or 6.2 to 7.5, or 6.5 to 9, or 6.5 to 8, or 6.5 to 7.5). In some embodiments, however, no pH adjustment may be necessary; simply taking up the de-starched com material in water may provide an aqueous mixture with a desired pH.

In various embodiments, as described above, the extraction pH is highly basic. For example, in various embodiments as otherwise described herein, the extraction pH is In various embodiments, the extraction pH is in the range of 10-14, for example, 10-13.7, or 10-13.5, or 10-13.3, or 10-13, or 10-12.7, or 10-12.5, or 10-12.3, or 10-12, or 10-11.7, or 10-11.5, or 10-11.3, or 10-11. In various embodiments, the extraction pH is in the range of 10.3-14, for example, 10.3-13.7, or 10.3-13.5, or 10.3-13.3, or 10.3-13, or 10.3-12.7, or 10.3-12.5, or 10.3-12.3, or 10.3-12, or 10.3-11.7, or 10.3-11.5, or 10.3-11.3, or 10.3-11. In various embodiments, the extraction pH is in the range of 11.5-14, for example, 11.5-13.7, or 11.5-13.5, or 11.5-13.3, or 11.5-13, or 11.5-12.7, or 11.5-12.5, or 11.5-12.3, or 11.5-12, or 11.5-11.7, or 11.5-11.5, or 11.5-11.3, or 11.5-11. In various embodiments, the extraction pH is in the range of 10.7-14, for example, 10.7-13.7, or 10.7-13.5, or 10.7-13.3, or 10.7-13, or 10.7-12.7, or 10.7-12.5, or 10.7-12.3, or 10.7-12, or 10.7-11.7, or 10.7-11.5, or 10.7-11.3. In various embodiments, the extraction pH is in the range of 11-14, for example, 11-13.7, or 11-13.5, or 11-13.3, or 11-13, or 11-12.7, or 11-12.5, or 11-12.3, or 11-12, or 11-11.7, or 11-11.5. In various embodiments, the extraction pH is in the range of 11.3-14, for example, 11.3-13.7, or 11.3-13.5, or 11.3-13.3, or 11.3-13, or 11.3-12.7, or 11.3-12.5, or 11.3-12.3, or 11.3-12, or 11.3-11.7. In various embodiments, the extraction pH is in the range of 11.5-14, for example, 11.5-13.7, or 10-13.5, or 11.5-13.3, or 11.5-13, or 11.5-12.7, or 11.5-12.5, or 11.5-12.3, or 11.5-12. In various embodiments, the extraction pH is in the range of 11.7-14, for example, 11.7-13.7, or 10-13.5, or 11.7-13.3, or 11.7-13, or 11.7-12.7, or 11.7-12.5, or 11.7-12.3.

In some embodiments, acids or bases can be used to adjust the pH of the aqueous mixture. These can be in concentrated form, or in the form of an aqueous medium (e.g., an acidic or alkaline solution) having a pH in the range of 5-9. For example, an acid (e.g., having a pH at most 4.9) or a base (e.g., having a pH of at least 9.1) may be added to provide the aqueous mixture at a desired pH of the de-starched corn material or during the maintaining step. Such acidic aqueous media may include organic as well as inorganic acids such as phosphoric acid, hydrochloric acid, citric acid, malic acid, acetic acid, other carboxylic acids, and combinations thereof. Such basic aqueous media may include inorganic bases such as ammonium hydroxide, sodium hydroxide, sodium carbonate and the like, and combinations thereof. In some embodiments, the one or more pH adjustments are performed by the addition of an alkaline solid. For example, the solid may be selected from sodium hydroxide, calcium hydroxide, or potassium hydroxide. In various embodiments, no catalytic agent is added to provide the aqueous mixture of the de-starched corn material or during the maintaining step. In some embodiments, ethanol and/or hydrogen peroxide are present in the aqueous mixture of the de-starched com material or during the maintaining step.

As described above, the aqueous mixture of the de-starched corn fiber at the extraction pH is heated for a time and at a temperature effective to extract arabinoxylan from the de-starched corn fiber into an aqueous phase of the aqueous mixture. In some embodiments of the present disclosure as described herein, the maintaining step is conducted for a time of at least 1 hour. For example, the maintaining step may in some embodiments be conducted for a time of at least 2 hours or at least 3 hours. In some embodiments of the present disclosure, the maintaining is conducted for a time in the range of 1 to 6 hours. For example, the maintaining step may be conducted for a time in the range of 1 to 4 hours, or 2 to 6 hours, or 2 to 4 hours, or 3 to 6 hours. In some embodiments of the present disclosure as described herein, the maintaining step is conducted at a temperature in the range of 70° C. to 150° C. For example, the maintaining step may be conducted at a temperature in the range of 70° C. to 140° C., or 70° C. to 130° C., or 70° C. to 120° C., or 70° C. to 110° C., or 70° C. to 100° C., or 80° C. to 150° C., or 80° C. to 140° C., or 80° C. to 130° C., or 80° C. to 120° C., or 80° C. to 110° C., or 80° C. to 100° C., or 90° C. to 150° C., or 90° C. to 140° C., or 90° C. to 130° C., to 90° C. to 120° C., or 90° C. to 110° C., or 90° C. to 100° C. The person of ordinary skill in the art will appreciate that maintaining under pressure can provide for temperatures higher than 100° C. to be reached. The person of ordinary skill in the art can provide heat and/or insulation to a vessel containing the material in order to maintain the desired temperature.

However, the present inventors have noted that extremes of pressure and temperature are not always necessary to provide a desirable product. Accordingly, in various embodiments as otherwise described herein, the maintaining step of the extraction is performed at a pressure of no more than 1.1 atmosphere, e.g., no more than 1.05 atmosphere. In various embodiments, the maintaining step of the extraction is performed at a temperature of no more than 100° C.

In some embodiments as described herein, after maintaining, the method further comprises adjusting the pH of the mixture. For example, in some embodiments, the method further comprises adjusting the pH of the aqueous mixture to a pH in the range of 7 to 10, e.g., in the range of 7 to 9.5, or 7 to 9.2, or 7 to 9, after maintaining. The pH adjustment can be accomplished as described herein.

As described above, the method also includes separating the aqueous phase containing the extracted arabinoxylan from the corn residue. The method of separation is not particularly limited and can be selected from any solid-liquid separation process known to those skilled in the art. For example, the separation step may be accomplished by filtration, centrifugation, or combinations thereof. In particular embodiments of the present disclosure as described herein, the separating step is accomplished by filtration. In particular embodiments of the present disclosure as described herein, the separating step is accomplished by filtration and centrifugation.

In some embodiments, after the separating step, the extracted arabinoxylan is further treated. For example, in some embodiments after the separating step, the extracted arabinoxylan is filtered (e.g., via nanofiltration and/or diafiltration). In various embodiments as described herein, the extracted arabinoxylan is filtered with a filter having a molecular weight cut-off of at least 0.5 kDa (e.g., at least 1 kDa).

The separated aqueous phase has extracted arabinoxylan extracted therein. As described above, the method also includes providing the arabinoxylan product from the aqueous phase. For example, in some embodiments, providing the arabinoxylan product from the aqueous phase comprises concentrating the aqueous phase containing the extracted arabinoxylan oligosaccharide. Concentrating the aqueous phase containing the extracted arabinoxylan can be accomplished by any method as known to the person of ordinary skill in the art. For example, concentrating the aqueous phase containing the extracted arabinoxylan may be accomplished by evaporation of water from the separated aqueous phase to provide an arabinoxylan product with a desired degree of solids, e.g., as a syrup or as a solid. The evaporation of the solvent may be accomplished by any method as known in the art, for example, spray drying, freeze drying, rotary evaporation, distillation, etc. In particular embodiments of the disclosure as described herein, providing the arabinoxylan product from the aqueous phase comprises spray-drying the aqueous phase containing the extracted arabinoxylan oligosaccharide. In some embodiments of the present disclosure as described herein, providing the arabinoxylan product from the aqueous phase comprises evaporation only to the extent needed to provide a more concentrated aqueous phase containing the extracted arabinoxylan oligosaccharides (e.g., a syrup). In other embodiments, the aqueous phase is evaporated to provide a solid; further drying of a solid, such as oven drying or drying with a stream of air or other gas (optionally heated) may also be used.

In some embodiments of the present disclosure as described herein, the arabinoxylan product is provided as a solid. For example, in various embodiments the arabinoxylan product is a solid having a total dry solids content in an amount of at least 90% by weight (e.g., at least 95 wt %). In other embodiments of the present disclosure as otherwise described herein, the arabinoxylan product is provided as a syrup. For example, in some embodiments of the present disclosure, the arabinoxylan product is a syrup having a total dry solids content in an amount of at least 50% by weight (e.g., at least 60%, or at least 70%, or at least 80% by weight). In various embodiments as otherwise described herein, the arabinoxylan product is a syrup having a total dry solids content in an amount in the range of 50-90% by weight, e.g., in the range of 50-85%, or 50-80%, or 50-75%, or 50-80%, or 55-95%, or 55-90%, or 55-85%, or 55-80%, or 55-75%, or 60-95%, or 60-90%, or 60-85%, or 60-80%, or 60-75%, or 65-95%, or 65-90%, or 65-85%, or 65-80%, or 65-75% by weight.

In some embodiments of the disclosure as described herein, the arabinoxylan product is provided as an emulsion (e.g., an oil-in-water emulsion). For example, in some embodiments, the emulsion comprises an aqueous phase comprising the arabinoxylan product and an oil. The aqueous phase and oil may be present in a weight ratio of at least 1:1 (e.g., at least 2:1, or at least 3:1). In some embodiments, the aqueous phase of the emulsion comprising the arabinoxylan product has a pH of at least 3 (e.g., at least 3.2, at least 3.5, or at least 4). In some embodiments, the aqueous phase of the emulsion comprising the arabinoxylan product has a pH in the range of 3-8 (e.g., in the range of 3-7, or 3-6, or 4-8, or 4-7, or 4-6). In some embodiments, the aqueous phase of the emulsion comprises the arabinoxylan product in an amount of at least 3% degree of solids (e.g., at least 3.5% degree of solids, at least 4.0% degree of solids, or at least 4.5% degree of solids). In some embodiments, the aqueous phase of the emulsion comprises the arabinoxylan product in an amount of no more than 10% degree of solids (e.g., no more than 8% degree of solids, or no more than 5% degree of solids).

The arabinoxylan product can be provided with a variety of molecular weights. In various embodiments of the present disclosure as described herein, the arabinoxylan product has a number average molecule weight (Mₙ) in the range of 2000 to 6000 g/mol. For example, in some embodiments, the arabinoxylan product has a number average molecule weight (Mₙ) in the range of 3000 to 6000 g/mol, or 4000 to 6000 g/mol, or 2000 to 5000 g/mol, or 3000 to 5000 g/mol. In some embodiments of the present disclosure as described herein, the arabinoxylan product has a weight average molecular weight (M_{w}) in the range of 50,000 to 400,000 g/mol. For example, in some embodiments the arabinoxylan product has a weight average molecular weight (Mw) in the range of 100,000 to 400,000 g/mol, or 150,000 to 400,000 g/mol, or 200,000 to 400,000 g/mol, or 50,000 to 300,000 g/mol, or 100,000 to 300,000 g/mol, or 150,000 to 300,000 g/mol, or 200,000 to 300,000 g/mol, or 50,000 to 200,000 g/mol, or 100,000 or 200,000 g/mol, or 150,000 or 200,000 g/mol. In some embodiments of the present disclosure as described herein, the extracted arabinoxylan oligosaccharides have a polydispersity in the range of 5-25. For example, in various embodiments, the arabinoxylan product has a polydispersity in the range of 8-25, or 10-25, or 12-25, or 15-25, or 5-20, or 8-20, or 10-20, or 15-20. Molecular weights are determined using liquid chromatography using pullulan as standards.

Notably, the processes described herein can be performed so as to provide a desirable arabinoxylan product without the need for further treatment or purification. In some embodiments of the present disclosure as described herein, no further cleaning steps are performed after the separating step. For example, the extracted arabinoxylan oligosaccharides are not further treated with acid or base after the separating step. In other embodiments, the extracted arabinoxylan oligosaccharides are not further washed with an aqueous medium after the separating step. In some embodiments of the present disclosure as described herein, the extracted arabinoxylan oligosaccharide are not subjected to further ion exchange processes or activated carbon treatment processes after the separating step.

However, in some embodiments of the present disclosure as described herein, additional treatment steps can be performed on the arabinoxylan product that is provided from the separated aqueous phase. For example, hydrolysis or condensation can be used to adjust the molecular weight of the material, e.g., in the presence of a catalyst. A variety of catalysts can be used, e.g., acids, bases, and enzymes (e.g., amylases, glucoamylases). The water content of the reaction medium can be adjusted to provide for overall condensation and molecular weight increase (i.e., at lower water contents) or to provide for overall hydrolysis and molecular weight reduction (i.e., at higher water contents). In other embodiments, after the separating step, the extracted arabinoxylan is filtered. The person of ordinary skill in the art can adjust reaction temperature, time and catalyst concentration to provide a desired degree of polymerization.

And in some embodiments, ion exchange processes or activated carbon treatment processes can be used, e.g., on the separated aqueous phase, to remove color and impurities from the material.

In some embodiments of the disclosure as described herein, the method further comprises treating the arabinoxylan product to decrease its viscosity. For example, in some embodiments, treating the arabinoxylan product comprises contacting the arabinoxylan product with one or more enzymes in a second enzyme treatment aqueous medium. In some embodiments of the present disclosure as described herein, the one or more enzymes include one or more amylases, one or more glucoamylases, one or more xylanases and/or one or more cellulases. For example, in some embodiments, the one or more enzymes include one or more endo-xylanases, such as Endo-xylanases SHEARZYME® from Novozymes. In various embodiments, treating the arabinoxylan product comprises contacting the arabinoxylan product with one or more enzymes in a second enzyme treatment aqueous medium is conducted for a time of at least 15 minutes (e.g., at least 30 minutes, at least 45 minutes, or at least 60 minutes). In various embodiments, treating the arabinoxylan product comprises contacting the arabinoxylan product with one or more enzymes in a second enzyme treatment aqueous medium is conducted for a time of at least 2 hours (e.g., at least 3 hours, at least 4 hours, or at least 5 hours). In various embodiments, treating the arabinoxylan product comprises contacting the arabinoxylan product with one or more enzymes in a second enzyme treatment aqueous medium is conducted for a temperature in the range of 25° C. to 70° C. (e.g., in the range of 25° C. to 60° C., or 30° C. to 70° C., or 30° C. to 60° C.). In various embodiments, treating the arabinoxylan product comprises contacting the arabinoxylan product with one or more enzymes in a second enzyme treatment aqueous medium is conducted at a pH in the range of 3 to 9, (e.g. in the range of 3 to 8, or 3 to 7, or 4 to 9, or 4 to 8, or 4 to 7).

The processes can be used herein to provide a high-fiber product. For example, in some embodiments of the methods as described herein, the arabinoxylan product has a dietary fiber content of 60-95% by weight on a dry solids basis; a digestible carbohydrate content of less than 20% by weight on a dry solids basis; a protein content of less than 15% by weight on a dry solids basis; and a fat content of less than 10% by weight on a dry solids basis.

Another aspect of the present disclosure as described herein provides an arabinoxylan product as prepared by the methods as described herein. For example, in some embodiments of the present disclosure, the arabinoxylan product has a dietary fiber content of 60-95% by weight on a dry solids basis; a digestible carbohydrate content of less than 20% by weight on a dry solids basis; a protein content of less than 15% by weight on a dry solids basis; and a fat content of less than 10% by weight on a dry solids basis.

As described above, the arabinoxylan product includes dietary fiber. In some embodiments, the arabinoxylan product has a dietary fiber content of 60-90% by weight on a dry solids basis. For example, in various embodiments the dietary fiber content is 60-85%, or 60-80%, or 65-85%, or 65-80%, 70-85%, or 70-80%, or 75-90%, or 75-85%, or 75-80%, or 80-90%, or 80-85% by weight on a dry solids basis. As used herein, dietary fiber is quantified using the standard AOAC 2009.01 method.

The arabinoxylan product desirably has a low digestible carbohydrate content. For example, in some embodiments of the present disclosure, the arabinoxylan product has a digestible carbohydrate content of less than 15% (e.g., less than 12%, or less than 10%) by weight on a dry solids basis. As used herein, digestible carbohydrate is quantified as the “available carbohydrate” as described in B. V. McCleary et al., “Measurement of available carbohydrates, digestible, and resistant starch in food ingredients and products,” Cereal Chemistry, 97(1), 114-137 (2019), available at https://onlinelibrary.wiley.com/doi/full/10.1002/cche.10208, which is hereby incorporated herein by reference in its entirety.

In some embodiments of the present disclosure as described herein, a ratio of dietary fiber to digestible carbohydrate is at least 4:1 (e.g., at least 5:1, or 6:1, or 7:1, or 8:1) by weight on a dry solids basis. For example, in some embodiments, the dietary fiber and digestible carbohydrate are present in a ratio in the range of 4-20:1 (e.g., in the range of 5-20:1, or 7-20:1, or 4-10:1, or 5-10:1, or 7-10:1).

As described above, the arabinoxylan product also includes protein and fat. For example, in some embodiments of the present disclosure as described herein, the protein is present in an amount of less than 12% (e.g., less than 8% or less than 5%) by weight on a dry solids basis. In other embodiments, the protein is present in an amount in the range of 1-15% by weight on a dry solids basis. For example, in some embodiments, the protein is present in an amount in the range of 1-12%, or 1-10%, or 1-8%, or 1-6%, or 1-4%, or 2-8% or 2-6%, or 2-4% by weight on a dry solids basis. In some embodiments of the present disclosure, the fat is present in an amount of less than 8% (e.g., less than 5%, or 3%, or 2%) by weight on a dry solids basis. In some embodiments of the present disclosure as described herein, the protein and fat are present in a combined amount of less than 15% (e.g., less than 10%) by weight on a dry solids basis. Protein is determined using the Kjeldahl method, using a conversion factor of 6.25 to convert Total Kjeldahl nitrogen to protein. Fat is determined using the method described in “Determination of Crude Oils and Fats,” Official Journal of the European Communities, No. 15/29, 18.1.84.

In some embodiments as described herein, the arabinoxylan product comprises an ash content of less than 10% by weight. For example, in some embodiments, the ash content is less than 8% by weight, or less than 5% by weight, or less than 3% by weight.

In some embodiments as described herein, the arabinoxylan product comprises arabinose, xylose, galactose, and glucose. In various embodiments as described herein, the arabinoxylan product comprises arabinose in an amount of no more than 50% by weight, no more than 45% by weight, or no more than 40% by weight, on a dry solids basis. In various embodiments as described herein, the arabinoxylan product comprises xylose in an amount of no more than 50% by weight, no more than 45% by weight, or no more than 40% by weight, on a dry solids basis. In various embodiments as described herein, the arabinoxylan product comprises galactose in an amount of less than 5% by weight, or less than 4.5% by weight, less than 4% by weight, or less than 3.5% by weight, or less than 3% by weight, on a dry solids basis. In various embodiments as described herein, the arabinoxylan product comprises glucose in an amount of less than 5% by weight, or less than 4% by weight, or less than 3% by weight, or less than 2% by weight, on a dry solids basis.

In some embodiments as described herein, the arabinoxylan product comprises arabinose in the range of 20-50% by weight on a dry solids basis. For example, in various embodiments, the arabinoxylan product comprises arabinose in the range of 20-45% by weight, or 20-40% by weight, or 20-35% by weight, or 25-50% by weight, or 25-45% by weight, or 25-40% by weight, or 25-35% by weight, on a dry solids basis. In some embodiments as described herein, the arabinoxylan product comprises xylose in the range of 20-50% by weight on a dry solids basis. For example, in various embodiments, the arabinoxylan product comprises xylose in the range of 20-45% by weight, or 20-40% by weight, or 20-35% by weight, or 25-50% by weight, or 25-45% by weight, or 25-40% by weight, or 25-35% by weight, on a dry solids basis.

In some embodiments as described herein, the arabinoxylan product comprises xylose and arabinose in a weight ratio of no more than 1:5:1 (e.g., no more than 1.4:1, or no more than 1:3:1, or no more than 1.2:1, or no more than 1.1:1) on dry solids basis. For example, in various embodiments, the arabinoxylan product comprises xylose and arabinose in weight ratio in the range of 1:1 to 1.5:1, or 1:1 to 1.4:1, or 1:1 to 1.3:1, or 1:1 to 1.2:1, or 1:1 to 1.1:1, on a dry solids basis.

In some embodiments as described herein, the arabinoxylan product comprises ferulic acid. For example, in various embodiments, the ferulic acid is present in an amount of at least 500 μg/g of arabinoxylan product, at least 1,000 μg/g of arabinoxylan product, or at least 5,000 μg/g of arabinoxylan product. In various embodiments, the ferulic acid is present in an amount of no more than 15,000 μg/g of arabinoxylan product, or 10,000 μg/g of arabinoxylan product, or no more than 7,500 μg/g of arabinoxylan product.

Another aspect of the present disclosure provides a method for making a food product, the method comprising providing an arabinoxylan product as described herein or an arabinoxylan product prepared by the method as described herein. Another aspect of the present disclosure provides a food product made by the method as described herein. Another aspect of the present disclosure provides a food product comprising an arabinoxylan product as described herein or an arabinoxylan product prepared by the method as described herein.

In some embodiments of the present disclosure as described herein, the food product comprises a confectionary composition. For example, the confectionary composition may be a chocolate composition, in which the arabinoxylan product is disposed. In some embodiments of the present disclosure as described herein, the food product is a candy, a bar (e.g., energy bar, snack bar, breakfast bar), a frozen dessert, or a baked good comprising the confectionary composition (e.g., chocolate composition, confectionary coating composition, enrobed baked good, or baked good with inclusions of chocolate or confectionary coating composition).

In other embodiments of the present disclosure as described herein, the food product is a fatty spread.

In some embodiments of the present disclosure as described herein, the food product is a cereal, a granola, a muesli, a topping, a coating, a baked good (e.g., cookie, a biscuit, a bread, a pastry, a pizza crust, a flatbread), a bar (e.g., snack bar, cereal bar, granola bar, energy bar), a meat alternative, a filling (e.g., a fruit filling or a crème filling), a fruit snack such as a fruit leather, a pasta, a sweetener, a frozen dessert, a dairy product (e.g., a yogurt, a quark, an ice cream), a dairy alternative product (e.g. yogurt alternative), a glaze, a frosting, a beverage, a syrup, a pet food, a medical food, a flavoring, or a dry blend. In other embodiments of the present disclosure as described herein, the food product is a meat alternative or a meat substitute.

In some embodiments of the present disclosure as described herein, the food product is a beverage. For example, the food product may be a dairy drink, a tea, a coffee, a water, a slimming beverage, a coarse grain drink, a fermented beverage, or a malted beverage.

The present inventors contemplate a number of particular uses for the arabinoxylan materials of the disclosure. As a soluble fiber with relatively low viscosity, the arabinoxylan product can be used in wide range of applications for fiber fortification, sugar and calorie reduction with affordable cost.

For example, the arabinoxylan product of the disclosure can be used in dairy drinks, such as dairy drinks with added fruit and cereal grains, dairy-based smoothies, yogurt, kefir, drinkable yogurt, long shelf life yogurt, dairy based meal replacement drinks, dairy-based drink mixes and any other dairy containing beverages. The arabinoxylan product can provide fiber fortification and potentially allow for low/no/reduced sugar claims, while providing enhanced mouthfeel in such beverages. Claims such as enhanced digestive health, weight management, and increase in satiety may be made through fiber addition.

As another example, the arabinoxylan product of the disclosure can be used in ready-to-drink, teas and coffees, tea and coffee drink mixes and textured teas or coffees. The arabinoxylan product can provide fiber fortification and potentially allow for low/no/reduced sugar claims, while providing enhanced mouthfeel in such beverages or textured teas/coffees. Claims such as enhanced digestive health may be made through fiber addition.

As another example, the arabinoxylan product of the disclosure can be used in juices. Juices are an example of a category that consumers already link to digestive health; such claims can be achieved through fiber addition. This product can be used in fruit and vegetable juices, e.g., blended juices, purees, and coulis.

As another example, the arabinoxylan product of the disclosure can be used in dairy alternatives such as nut milks, oat milk, dairy-free beverage mixes, cereal/grain drinks, almond milks, rice, cashew, soy milk, hemp milk, and coconut milk. Traditionally these products can be perceive as being ‘thin’ or not having the same texture/mouthfeel as more traditional products. Fiber addition can deliver benefits both in terms of texture/mouthfeel as well as digestive health benefits.

As another example, the arabinoxylan product of the disclosure can be used in waters, flavored/or unflavored, and sparkling or still. Fiber can be added to these drinks for enhanced digestive health claims and adjustment of texture/mouthfeel.

As another example, the arabinoxylan product of the disclosure can be used in slimming beverages. This arabinoxylan product can be used to make claims related to weight management/slimming benefits, increase in satiety and enhanced digestive health.

As another example, the arabinoxylan product of the disclosure can be used in coarse grain drinks/food products, such as beverages made with coarse grains, coarse grain drink mixes/powders, and coarse grain drinks combined with other beverages.

As another example, the arabinoxylan product of the disclosure can be used in fermented beverages such as beer, wine, kombucha, sauerkraut juice, mead, and rice wine.

A variety of other examples include: use as a binding agent; use in cereals, granola, and muesli; use in cereal bars, baked bars, and nut clusters; use in snack toppings and cereal toppings, for example in yoghurts and parfaits; use in coatings and/or extruded cereals; use in cookies, biscuits and shortbread; use in bread and baked goods, such as in a sugar base (e.g., in scones, muffins, strudels, cakes) and in products such as pizza crusts, flatbreads, naan breads; use in pasta; use in mixes such as for cake, muffins and break; use as sweetener in nut milk-based drinks; use in breakfast/energy bars, e.g., soft baked bars, crunchy bars, protein bars, fiber bars; use in aerated protein bars/bites, e.g., to create texture and foam; use in meat alternatives such as vegan fillets, and mock meats, for example to provide texturizing; use in fillings for sandwich cookies; use in fruit snacks and leathers to provide a desired texture, which can be controlled using molecular weight; use in soups, sauces and dressings, to provide consistency and texture based on desired viscosity profiles; use in meal replacement applications; use in ice creams, frozen yogurts and frozen desserts; use in frostings and glazes; use in malted beverages; use in flavored syrups; use in pet foods and pet treats; use in ingredient compositions such as spray dried flavors, dry blends, plated products; use in any confectionery applications, e.g., chocolates, chocolate truffles/bars/fillings, fondants; use in aerated products, chews, leathers, candies, and gummies; use in dietary supplements, e.g., in the form of powders, chews, gelcaps, tablets, and gummies; and use in fruit fillings, fruit preparations and flavored fruit snacks.

### EXAMPLES

The Examples that follow are illustrative of specific embodiments of the method of the disclosure, and various uses thereof. They are set forth for explanatory purposes only, and are not to be taken as limiting the scope of the disclosure.

### Example 1. Extraction of Arabinoxylan at Neutral, Basic, and Acidic pH

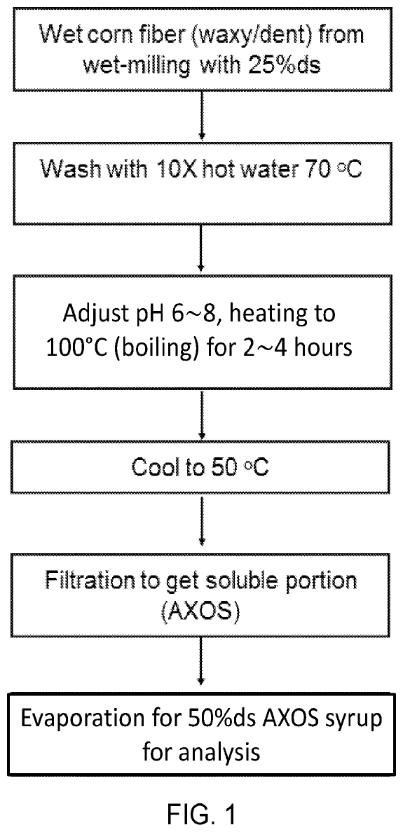

A wet com fiber obtained from a wet-milling process with 25% degree of solids was used as a starting material in a measure of the efficiency of extracting arabinoxylan under neutral pH. To de-starch the corn fiber, the wet corn fiber was washed with hot water at 70° C. at 10 times the mass on a dry solids basis of the wet corn fiber. The soluble products were removed and the solid de-starched corn fiber was used for the extraction process. The de-starched corn fiber was heated to 100° C. at a pH of 7 for 3 hours. The mixture was then cooled to 50° C. and filtered to isolate the aqueous phase of the de-starched corn fiber slurry. The aqueous phase was then evaporated to 50% degree solids for analysis. FIG. 1 provides a flowchart of this extraction process.

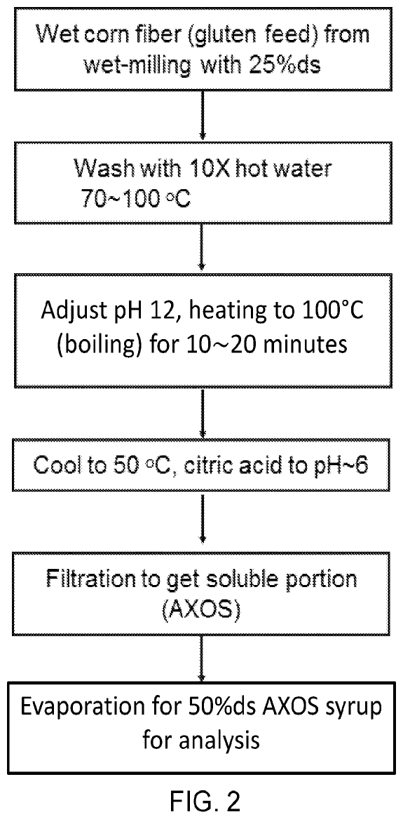

Similar procedures were followed to extract arabinoxylan under basic and acidic conditions as comparative examples. Specifically, for the extraction under basic conditions, a wet corn fiber obtained from a wet-milling process with 25% degree of solids was used. To de-starch the corn fiber, the wet corn fiber was washed with hot water at 70° C. at 10 times the mass on a dry solids basis of the wet corn fiber. The soluble products were removed and the de-starched corn fiber slurry was used for the extraction process under basic conditions. The de-starched corn fiber was heated to 100° C. at a pH of 12 for 10-20 minutes. The mixture was then cooled to 50° C. and the pH was adjusted to 6 using citric acid. The mixture was then filtered to isolate the aqueous phase of the de-starched corn fiber slurry. The aqueous phase was then evaporated to 50% degree of solids for analysis. FIG. 2 provides a flowchart of this extraction process under basic conditions.

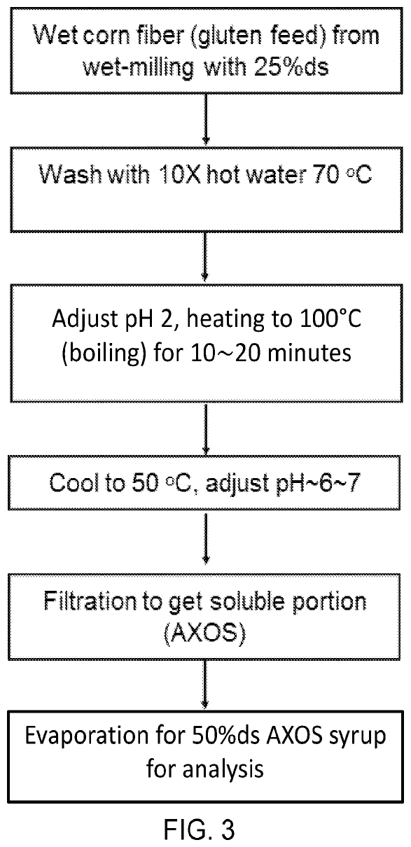

For the extraction under acidic conditions, again a wet corn fiber obtained from a wet-milling process with 25% degree of solids was used. To de-starch the com fiber, the wet corn fiber was washed with hot water at 70° C. at 10 times the mass on a dry solids basis of the wet com fiber. The soluble products were removed and the de-starched corn fiber slurry was used for the extraction process under basic conditions. The de-starched corn fiber was heated to 100° C. at a pH of 2 for 10-20 minutes. The mixture was then cooled to 50° C. and the pH was adjusted to between 6 and 7. The mixture was then filtered to isolate the aqueous phase of the de-starched com fiber slurry. The aqueous phase was then evaporated to 50% degree of solids for analysis. FIG. 3 provides a flowchart of this extraction process under acidic conditions.

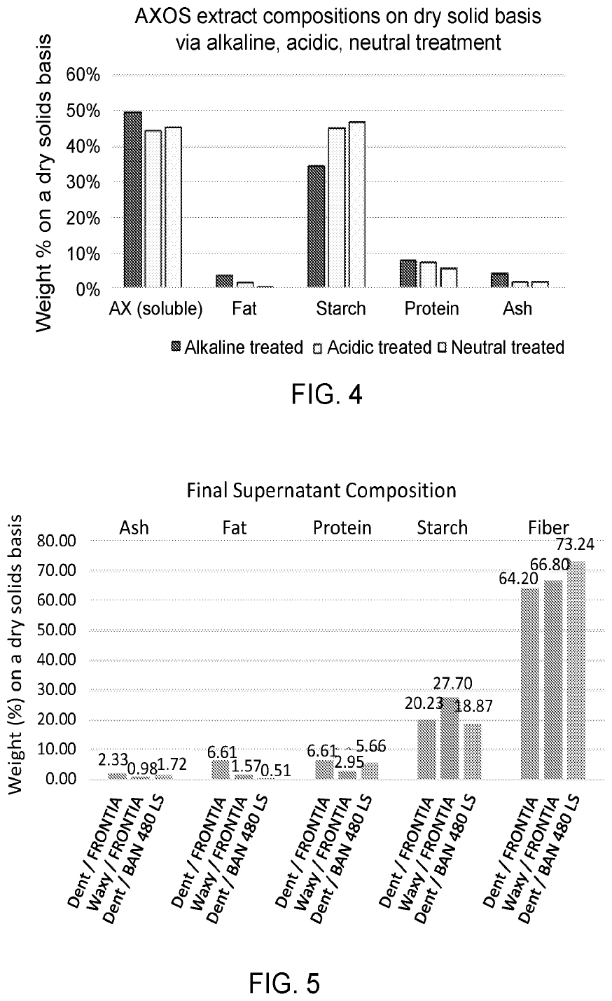

The resulting arabinoxylan containing syrup for these three extraction process where then analyzed for their arabinoxylan, fat, starch, protein, and ash content. These results are reported in FIG. 4. From FIG. 4, it is clear that all three extraction methods produced products with arabinoxylan content higher than 40% by weight on a dried solids basis, with the neutral and basic extraction processes providing more arabinoxylan than the acidic extraction. These results correspond to an 8% yield of the corn fiber on a dry solids basis, with approximately 30% arabinoxylan recovery. Residual starch was present in the products from all of the extraction processes with 34-47% by weight on a dry solid basis.

While the basic extraction product included the highest amount of arabinoxylan, it also contained the most ash due to the pH change. Conversely, the neutral pH had the least ash content. Both the neutral pH and acidic extraction products have a much lighter yellow color compared to the basic extraction product which appeared brown. Further, the neutral pH extraction product had the lowest amount of fat and protein present.

### Example 2. Extraction of Arabinoxylan at Neutral pH With Enzymatic De-Starching

A preliminary enzymatic de-starching was performed, followed by neutral pH treatment for 30 minutes vs. 3 hours to extract arabinoxylan from corn fiber. These experiments provide insights into the efficiency of the starch de-starching and the time necessary for the neutral pH extraction treatment.

For the extraction process with the enzyme, corn wet milling washed fiber (300 g of dry solid) was diluted in water to roughly 10% degree of solids. To the mixture of corn fiber, an a-amylase enzyme (BAN 480 LS, from Novozymes) was added under a pH of approximately 7 at 70° C. for 60 minutes. The mixture is then centrifuged to collect the de-starched insoluble phase for further extraction of arabinoxylan. The de-starched insoluble phase is evenly split into two beakers and heated to 100° C. under a pH of 6-8 with stirring. The first beaker was allowed to react for 3 hours, while the second beaker was reacted for 30 minutes. The pH of the insoluble phase reaction was continuously checked and water was added as needed to maintain a pH between 6 and 8. At the end of the reaction time, the mixture was cooled to 50° C. The soluble phase and insoluble by-product phase were separated by centrifugation, and the arabinoxylan product was isolated by concentrating the soluble phase using a rotary evaporator.

As a comparison, the hot water de-starching was performed, followed by neutral pH treatment for 3 hours. Specifically, corn wet milling washed fiber (150 g, dry solid) was diluted in water to roughly 10% degree of solids. The corn fiber mixture was heated to 70° C. at a pH ˜8 for 90 minutes to remove soluble starch, protein, and some organic acids. The cleaned insoluble material was then heated to 100° C. under a pH of 6-8 with stirring for 3 hours. The pH of the insoluble phase reaction was continuously checked and water was added as needed to maintain a pH between 6 and 8. At the end of the reaction time, the mixture was cooled to 50° C. The soluble phase and insoluble by-product phase were separated by centrifugation, and the arabinoxylan product was isolated by concentrating the soluble phase using a rotary evaporator.

### Example 3. Extraction of Arabinoxylan From Different Corn Fiber Sources at Neutral pH With Enzymatic De-Starching

Other sources of corn fiber as well as different de-starching enzymes were also evaluated. A dent corn fiber feed (before dewatering) and a waxy corn feed (before dewatering) were used with the FRONTIA® de-starching enzyme from Novozymes.

The dent corn fiber feed (at 15% degree of solids) was also used to track the starch release performance of FRONTIA®. Specifically, 300 g of the dent corn fiber was diluted with water to approximately 6% degree of solids. To the mixture, 0.04% w/w of FRONTIA® enzyme was added for de-starching at 50° C. and a pH of 4.3 for 1 hour. During the enzyme treatment, de-starched slurry samples were taken at different time points and separated to provide the de-starched insoluble phase. The de-starched insoluble phase was then collected and heated at 100° C. under a pH of 6-8 for 3 hours with stirring. At the end of the reaction time, the mixture was cooled to 50° C. The soluble phase and insoluble by-product phase were separated by centrifugation, and the arabinoxylan product was isolated by concentrating the soluble phase using a rotary evaporator.

The same procedure as described in the above paragraph was used to treat a dent corn fiber feed and a waxy corn fiber feed, except that de-starched slurry samples were not taken at different time points. Rather, the entire corn fiber slurry (either dent or waxy) was allowed to be de-starched for 1 hour. Following this enzymatic de-starching with FRONTIA®, both the de-starched dent corn fiber mixture and the de-starched waxy corn fiber mixture were subjected to arabinoxylan extraction under neutral pH (e.g., pH 6-8) for 3 hours, as described above. At the end of the reaction time, the mixture was cooled to 50° C. The soluble phase and insoluble by-product phase were separated, and the arabinoxylan product was isolated by concentrating the soluble phase using a rotary evaporator.

FIG. 5 shows the results of the analysis of the final supernatant composition prepared from the dent corn fiber feed and the waxy corn fiber feed. These results are also show to the final supernatant composition as prepared according to Example 2 (i.e., dent/BAN480 LS). The ash, fat, and protein content for all supernatants are less than 10 wt. %. while the starch content is greater than 15 wt. % and the fiber content is greater than 60 wt. %.

### Example 4. Improvements in FRONTIA® Enzymatic De-Starching for Arabinoxylan Extraction

The enzymatic extraction process was further investigated to improve upon the results shown in Example 3. Specifically, a corn wet milling washer fiber (300 g, dry solid) was diluted with water to obtain a mixture of approximately 15% degree of solids. To the mixture, 0.04% w/w of the FRONTIA® enzyme was added for de-starching at 50° C. and pH 4.3 for 1 hour. After the reaction, no centrifuge was applied. Rather, the soluble phase was separated using a spatula from the de-starched insoluble phase. The de-starched insoluble phase was then washed twice by equal amounts of water. The de-starched insoluble phase was then heated at 100° C. under a pH of 6-8 with stirring for either 1.5 hours or 3 hours. The pH was continuously checked during the reaction and maintained by adding water as needed. The soluble phase and insoluble by-product phase were separated by centrifugation, and the arabinoxylan product was isolated by concentrating the soluble phase using a rotary evaporator.

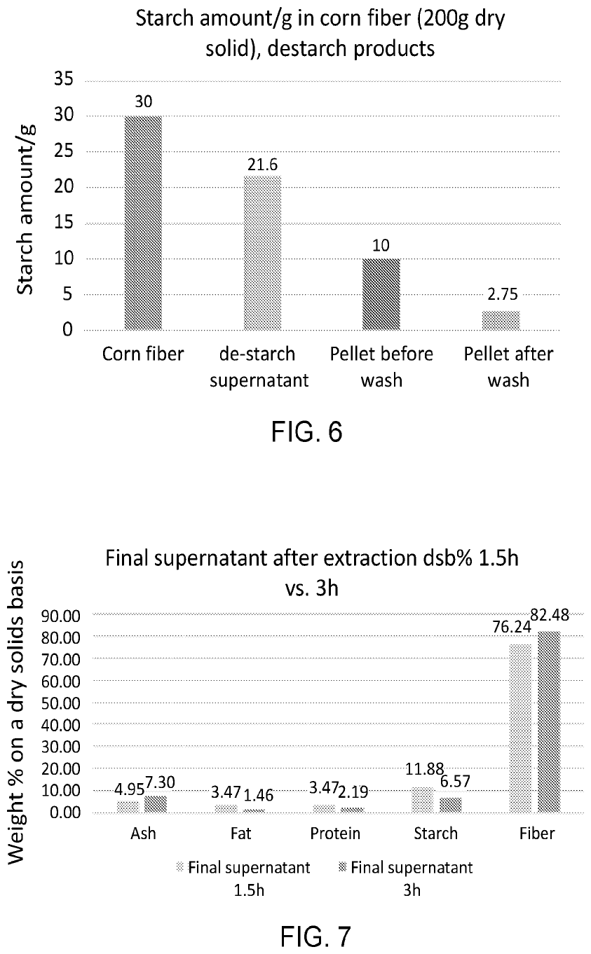

FIG. 6 compares the amount of starch in the corn fiber, the de-starched supernatant, the pellet before washing, and the pellet after watching. FIG. 6 shows that starch can continue to be removed after the FRONTIA® treatment by a water wash.

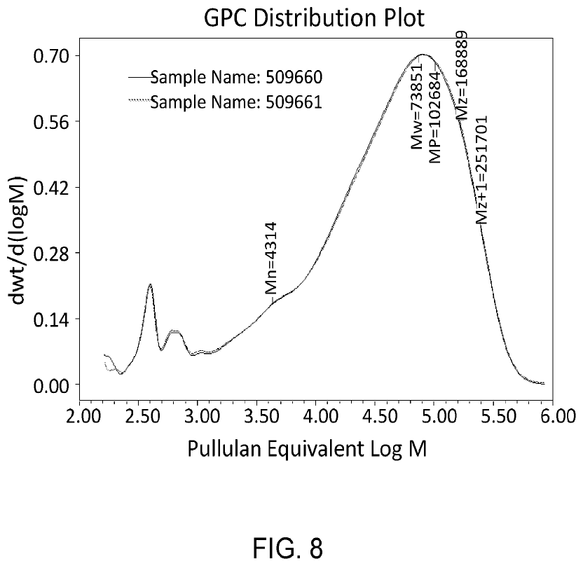

The final products for the neutral pH treatment of 1.5 and 3 hours as described above were then analyzed for their fiber, starch, protein, fat, and ash content, the results of which are shown in FIG. 7. The 3 hour treatment provided more fiber and less starch, protein, and fat than the 1.5 hour treatment. But the 1.5 hour treatment had the least amount of ash present in the final product. These final products for the 1.5 and 3 hour treatments were also analyzed with Gel Permeation Chromatography to analyze the arabinoxylan product obtained. Table 1 reports the GPC results and FIG. 8 shows the resulting chromatographs.

TABLE 1

| Sample | Mₙ (Pullulan | M_{w} (Pullulan | |
|---|---|---|---|
| Name | Equivalent Log M) | Equivalent Log M) | Polydispersity |
| Final | 4364 | 72313 | 16.6 |
| Product | | | |
| (1.5 hrs.) | | | |
| Final | 4469 | 76072 | 17 |
| Product | | | |
| (3 hrs.) | | | |

### Example 5. Concentration Dependence of FRONTIA® Enzymatic De-Starching for Arabinoxylan Extraction

The enzymatic extraction process was further evaluated to improve upon the results shown in Examples 3 and 4. Four trials (Trials 1-4) were performed to evaluate the effect of enzyme concentration on the de-starching process. Additionally, a control was measured that did not include any enzyme. For each trial, a waxy corn fiber (300 g, dry solid) was diluted to obtain a mixture that was approximately 17% degree of solids. To each trial mixture, different amounts of the FRONTIA® enzyme was added for de-starching at 45° C. and pH of 4.4 for 150 minutes. Table 2 shows the reaction parameters for Trials 1-4.

TABLE 2

| | Trial 1 | Trial 2 | Trial 3 | Trial 4 |
|---|---|---|---|---|
| Waxy corn fiber | 304.6 | 303.1 | 305.1 | 303.1 |
| (g) | | | | |
| Weight after | 802.2 | 804 | 804.2 | 802.1 |
| water addition | | | | |
| (g) | | | | |
| DS (g) | 51.8 | 51.5 | 51.9 | 51.5 |
| Enzyme dosage | 35 | 70 | 140 | 280 |
| (μL) | | | | |
| Enzyme dosage | 0.081% | 0.163% | 0.324% | 0.652% |
| (g/g DS) | | | | |
| Enzyme | 0.052 | 0.104 | 0.209 | 0.419 |
| concentration | | | | |
| (mg/g slurry) | | | | |
| Reaction time | 150 | 150 | 150 | 150 |
| (minutes) | | | | |

Each reaction slurry was then dived into two portions, A and B. Portion A was filtered through a 75 μm screen with no further washing and portion B was filtered and washed with 150 g of water. The wet de-starched fiber was then collected, weighed, and dried at 50° C. in an oven for approximately 12 hours. The de-starched dried fiber was then analyzed for fiber content.

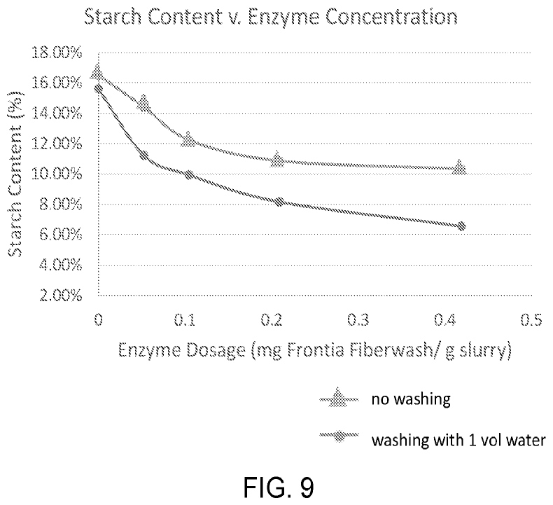

FIG. 9 compares the amount of starch in the de-starched fiber for Portions A and B of Trials 1-4. It is observed that the residual starch decreases with the increasing concentration of the FRONTIA® enzyme, and that the highest concentration in this test (0.418 mg enzyme/g slurry) reduces the residual starch from 16.5% (the original starting material) to 10.33%. Additional improvement is observed when washing with 1 volume of water (i.e., the amount of water with the same weight of the starting crude fiber), in this case 150 g of water.

### Example 6. FRONTIA® Enzymatic De-Starching for Arabinoxylan Extraction With Washing Step

As described in Example 5, de-starching of the fiber was improved with increased enzyme concentration and with an additional washing step after enzymatic de-starching. To further investigate the effects of the washing step, four more trials (Trials 5-8) were conducted. The same de-starching procedure as described in Example 5 were used in this Example. Table 3 shows the reaction parameters of Trials 5-8.

TABLE 3

| | Trial 5 | Trial 6 | Trial 7 | Trial 8 |
|---|---|---|---|---|
| Waxy corn fiber | 302.1 | 303.4 | 304.25 | 400.6 |
| (g) | | | | |
| Weight after | 801 | 814 | 803.4 | 802.4 |
| water addition | | | | |
| (g) | | | | |
| DS (g) | 41.4 | 51.6 | 51.7 | 68.1 |
| Enzyme dosage | 280 | 140 | 280 | 280 |
| (μL) | | | | |
| Enzyme dosage | 0.654% | 0.326% | 0.650% | 0.493% |
| (g/g DS) | | | | |
| Enzyme | 0.419 | 0.206 | 0.418 | 0.419 |
| concentration | | | | |
| (mg/g slurry) | | | | |
| Reaction time | 150 | 240 | 240 | 240 |
| (minutes) | | | | |

Trials 5 and 7 are the same conditions except for the reaction time, and Trial 6 is run at half the enzyme concentration of Trial 5 but with increased reaction time. Trials 7 and 8 are run at the same conditions expect for the solids content of the starting material.

In each of these trials, as with Example 5, the reaction slurry was then dived into two portions, A and B. In this case, Portion A was filtered with no further washing and Portion B was filtered and washed with 400 g of water, for Trials 5-7, and 600 g of water for Trial 8. The wet de-starched fiber was then collected, weighed, and dried at 50° C. in an oven for approximately 12 hours. The de-starched dried fiber was then analyzed for fiber content.

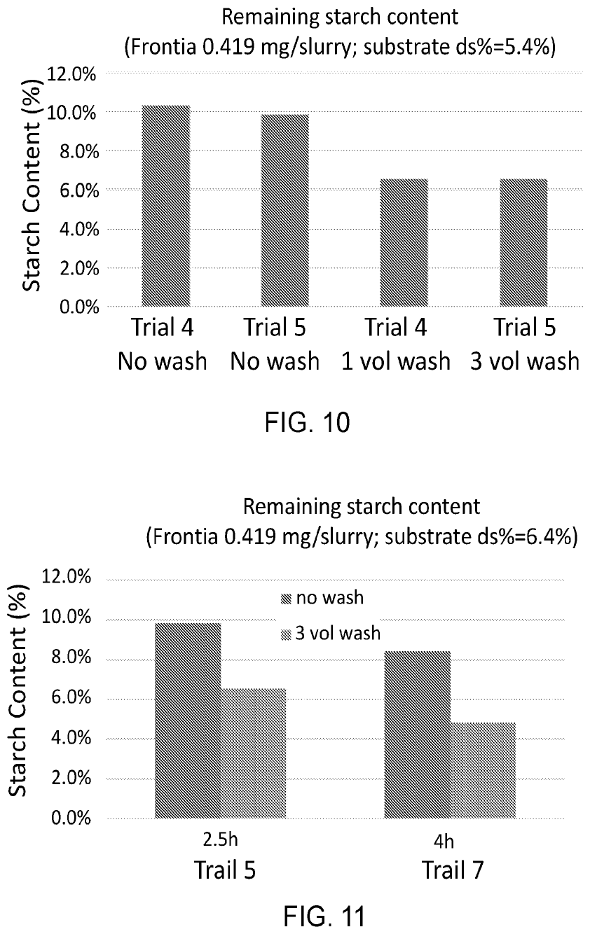

FIG. 10 shows the impact of the increased volume of washing material by comparing the starch content of Trial 4 of Example 5 and Trial 5 of Example 6. Portion B of Trial 5 was washed with 3 volumes of water instead of 1 volume as in Trial 4. However, the residual starch content in these two sample are similar (6.6% vs. 6.5%).

FIG. 11 shows the impact of reaction time by comparing the starch content of Trial 5 and Trial 7. Trial 5 was run for 2.5 hours and Trial 7 was run for 4 hours. From the graph of FIG. 11, it is observed that running the enzymatic de-starching for 4 hour further decreases the starch content when compared to treatment for 2.5 hours. For Portion A, without washing, the increased reaction time decreased the starch content to 8.4% from 9.87%, and for Portion B, with washing (3 volumes); the increased reaction time decreased the starch content from 6.53% to 4.8%.

Additionally, the de-starched products of Trials 5-8 as well as the starting material (as described in Example 5) were analyzed for their protein, fat, and ash content. The results are shown in Table 4.

TABLE 4

| | Ash | Protein | Fat | Starch | Total | Non-starch |
|---|---|---|---|---|---|---|
| | (%) | (%) | (%) | (%) | carbohydrate | carbohydrate |
| Trial 5 | 0.72 | 10.57 | 5.11 | 6.53 | 80.6 | 74.0 |
| Trial 6 | 0.67 | 10.60 | 5.49 | 7.19 | 80.5 | 73.3 |
| Trial 7 | 0.66 | 9.94 | 4.76 | 4.82 | 82.1 | 77.3 |
| Trial 8 | 0.72 | 9.55 | 5.20 | 4.6 | 82.0 | 77.4 |
| Waxy corn fiber | 3.57 | 21.30 | 5.21 | 16.45 | 64.1 | 47.7 |
| (starting material) | | | | | | |

These de-starched fibers were able to be dried to lower moisture content compared to the original crude fiber. Without being bound by theory, the present inventors hypothesize that this is because starch is more moisture binding. The results of Table 4 show that the ash, protein and starch content are significantly lowered after the de-starching process.

### Example 7. FRONTIA® Enzymatic De-Starching for Arabinoxylan Extraction With Increased Enzyme Concentration

As described in Examples 5 and 6, de-starching of the fiber was improved with increased enzyme concentration, an additional washing step after enzymatic de-starching, and increased reaction time. To further investigate how much more starch can be removed when the enzyme concentration is further increased, two more trials (Trials 9 and 10) were tested using the same de-starching procedure as described in Example 5. Table 5 shows the reaction parameters of Trials 9 and 10.

TABLE 5

| | Trial 9 | Trial 10 |
|---|---|---|
| Waxy corn fiber (g) | 400.95 | 401.1 |
| Weight after water addition | 801.5 | 801.37 |
| (g) | | |
| DS (g) | 68.2 | 68.2 |
| Enzyme dosage (μL) | 560 | 1120 |
| Enzyme dosage (g/g DS) | 0.986% | 1.971% |
| Enzyme concentration | 0.838 | 1.677 |
| (mg/g slurry) | | |
| Reaction time (minutes) | 235 | 245 |

The enzyme concentration compared to that used for Trial 8 of Example 6 was doubled for Trial 9 and quadrupled for Trial 10. As with Example 6, the reaction slurry was then dived into two portions, A and B. In this case, Portion A was filtered with no further washing and Portion B was filtered and washed 600 g of water. The wet de-starched fiber was then collected, weighed, and dried at 50° C. in an oven for approximately 12 hours. The de-starched dried fiber was then analyzed for fiber content.

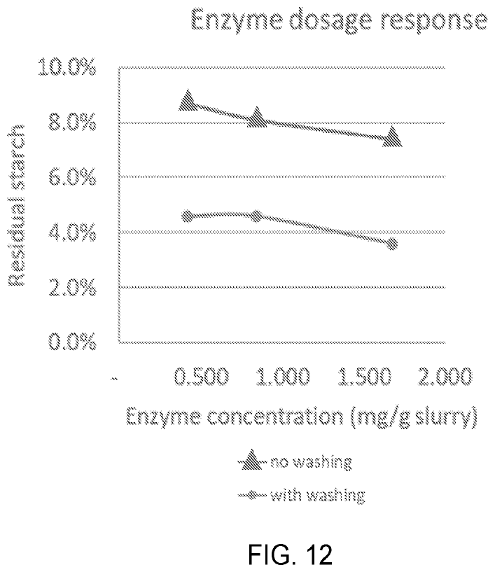

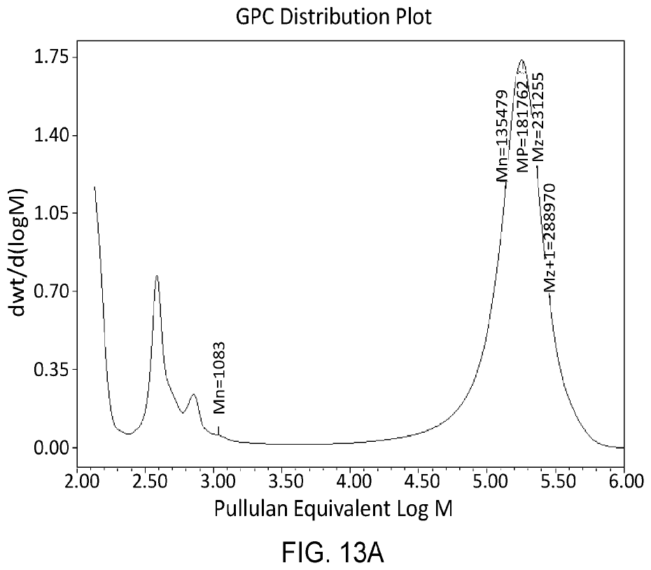

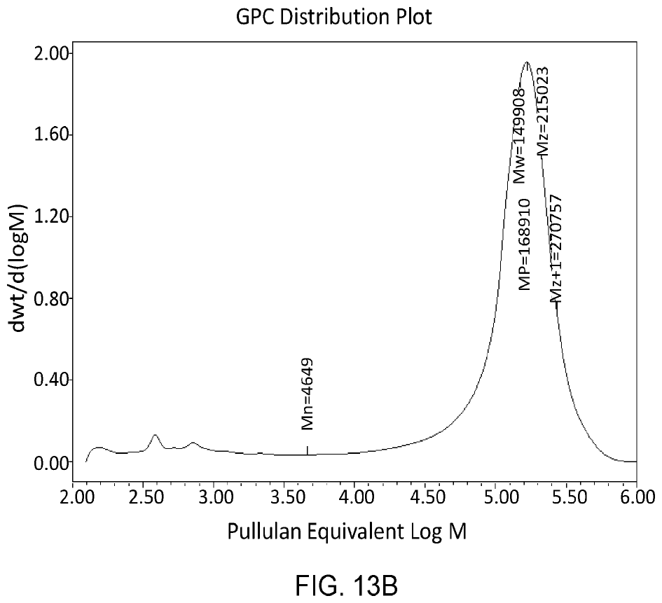

FIG. 12 shows the residual starch from Trials 8 of Example 6, and Trials 9 and 10. Doubling the enzyme concentration from 0.419 mg enzyme/g slurry (Trial 8) to 0.838 mg enzyme/g slurry (Trial 9) resulted in a starch decrease from 8.7% to 8.10% with no washing, and now difference with washing. Further quadrupling the enzyme concentration does not provide very significant improvements.

Overall, from Examples 5-7, increasing the FRONTIA® enzyme concentration, additional washing after the enzymatic de-starching, and increasing the reaction time improved the starch removal from the corn fiber.

### Example 8. Effect of pH on Extraction of Arabinoxylan

Arabinoxylan was extracted from enzymatically de-starched fibers, as described in Examples 5-7 (with FRONTIA® enzymes), using extraction solutions at different pHs. To perform these experiments, 303 g of the de-starched fiber was weighed into a large beaker and 1124 g of water was added. The degree of solids of the de-starched fiber was 26%, providing a final degree of solids of the slurry of 6.4%. The slurry was then divided into three portions and the pH was adjusted to 8.8 (Trial 11), 7.5 (Trial 12), and 5.5 (Trial 13) with a 5% by volume NaOH solution.

Each sample was then heated with a 97° C. water bath and stirred, with overhead stirring, for 3 hours at 200 rpm. The samples were then removed from the water bath and the liquid portion was collected and centrifuged at 5000 rpm for 15 minutes. The supernatant was collected and the pH and Brix number of the supernatant were measured. Table 6 details the reaction conditions and the results for these three samples.

TABLE 6

| | Trial 11 | Trial 12 | Trial 13 |
|---|---|---|---|
| Beaker (g) | 198.26 | 197.5 | 213.4 |
| Slurry (9) | 401.98 | 406.5 | 410.98 |
| pH | 8.84 | 7.5 | 5.53 |
| 5% NaOH used (g) | 2.37 | 1.56 | 0 |
| Starting DS (g) | 25.7 | 26.0 | 26.3 |
| Weight of | 206 | 212 | 215 |
| supernatant (g) | | | |
| pH of supernatant | 6.84 | 6.66 | 5.24 |
| Brix | 0.7 | 0.7 | 0.5 |
| Extracted DS (g) | 1.442 | 1.484 | 1.075 |
| Crude yield based | 5.6% | 5.7% | 4.1% |
| on DS (g) | | | |
| Yield based on total | 11.7% | 11.9% | 8.5% |
| available AX | | | |

For Trials 11-13, a pH drop was observed in the supernatant, with the initially high pH (8.84) decreasing the most. The three supernatants of Trials 11-13 were then concentrated on a rotary evaporator and analyzed for their starch and protein content. These results are reported in Table 7 and are described in more detail below.

The extraction of Trials 11-13 were conducted at 97° C. at three different pHs. To further evaluate the effect of temperature on the extraction process, the same enzymatically de-starched fiber used above was formed into a slurry with 305 g of the wet fiber and 902 g of water. The pH of the slurry was adjusted from an initial pH of 5.6 to a final pH of 7.5 with a 5% by volume NaOH solution. A total of 2.92 g of the NaOH solution was used.

The pH adjusted slurry was then divided into 3 samples. Each sample was placed in a retort machine with the temperature set to 135° C. The reaction was conducted for 1 hour once the internal temperature reached 133° C. The samples were cooled down to room temperature and the materials were combined. The final pH of the combined sample was 5.3, a significant drop from the initial pH of 7.5. The soluble materials were collected after centrifugation at 5000 rpm for 15 minutes. A total of 476 g of supernatant with a Brix number of 0.8 was obtained. The total extracted dry solid product was 3.8 g, making the crude extraction yield 8.4% based on the amount of de-starch fiber of the sample. The extract was concentrated on a rotary evaporator and the composition was analyzed. These results are reported in Table 7.

TABLE 7

| | Trial 11 | Trial 12 | Trial 13 | Trial 14 |
|---|---|---|---|---|
| Extraction | 97° C., 3 hours, | 97° C., 3 hours, | 97° C., 3 hours, | 133° C., 1 |
| Conditions | initial pH 8.8 | initial pH 7.5 | initial pH 5.5 | hour, initial |
| | | | | pH 7.5 |
| ds % of the | 3.10% | 3.40% | 4.30% | 9.30% |
| concentrate | | | | |
| Crude Yield | 5.6% | 5.7% | 4.1% | 8.40% |
| based on DS of | | | | |
| destarched fiber | | | | |
| Starch % dsb | 39.0% | 40.0% | 47.2% | 25.2% |
| Protein % dsb | 14% | 12% | 12% | 6% |

From the results of Table 7, it is clear that arabinoxylan extract can be improved by increasing the extraction temperature as well as by increasing the pH.

### Example 9. Effect of Using Ground Corn Fiber on Arabinoxylan Extraction

In the previous trials of Examples 1-8, the corn fiber was de-starched without any other pre-extraction conditioning. In this Example, the effect of using a ground corn fiber on the arabinoxylan extraction was determined. As a comparison, the enzymatically de-starched fiber was used by first drying the fiber in an oven (at 50° C.) to provide an accurate dry solid weight. For the slurry, 33.26 g of dry fiber (degree of solids—72.15%) was diluted to 400 g (degree of solids—6.0%). Two parallel extractions were run at 97° C. for 3 hours with an initial pH adjusted to 8.9 with a 5% by volume NaOH solution (Trial 15) as described in Example 8.

After the reaction was complete, the samples were removed from the water bath and the two samples were combined and filtered through Whatman filter paper over a Büchner funnel. The filtrate (Trial 15A) was collected and the pH and Brix number were measured. The insoluble fiber in the funnel was washed with 400 g of water and collected (Trial 15B). The crude yield of these filtrates are reported in Table 8.

TABLE 8

| | | | Trial 15A & | Dried Solids of |
|---|---|---|---|---|
| | | | Trial 15B, | destarched |
| | Trial 15A | Trial 15B | combined | fiber (g) |
| Mass (g) | 313 | 408 | — | 48 |
| Brix (%) | 0.8 | 0.4 | — | — |
| DS collected (g) | 2.5 | 1.6 | — | — |
| pH | 6.65 | 7.27 | — | — |
| Crude Yield | 5.2% | 3.3 | 8.5 | — |

To provide the ground corn fiber, the enzymatically de-starched fiber was first dried in an oven at 50° C. and ground to a powder in a coffee grinder. Two slurries were formed at 6% degree of solids (Trial 16) and 10% degree of solids (Trial 17), respectively. For each of these trials, two parallel reactions were conducted. The reactions were performed at 97° C. for 3 hours, with an initial pH of 8.0. After the reaction was complete, the samples were combined. The final pH dropped to 6.6 for both trials. The soluble materials were collected after centrifugation at 5000 rpm for 15 minutes. The insoluble corn fiber was further washed with water to recover more soluble product. The total dry solid collected from Trial 16 was 6.0 g and from Trial 17 was 7.2 g. The crude yield of these trials were 12.5% (for Trial 16) and 9% (for Trial 17). This result indicates that the extraction efficiency was lower when starting at a higher solid level. However, compared to Trial 15A, Trial 15B, and the combination of Trials 15A and 15B described above, grinding the corn fiber prior to performing the extraction significantly increases the yield.

The combined Trials 15A and 15B and the extract of Trials 16 and 17 were concentrated on a rotary evaporator and analyzed. These results are reported in Table 9.

TABLE 9

| | Trials 15A & | | |
|---|---|---|---|
| | 15b, combined | Trial 16 | Trial 17 |
| Crude Yield | 8.5% | 12.5% | 9% |
| AX concentration | 8.1% ds | 8.4% ds | 11.9% ds |
| mc % | 91.90% | 91.60% | 88.10% |
| Starch % dsb | 34.4% | 28.5% | 45.1% |
| Protein % dsb | 5.3% | 13.6% | 11.8% |

### Example 10. Alkaline Extraction of Arabinoxylan

Trials 18 and 19 were performed to evaluate whether increasing the initial pH of the extraction would increase the extraction yield. For Trial 18, the residual fiber from Trial 16 was dried in an oven overnight and 24 g (dried solids basis) was diluted in water to 400 g, providing a 5% degree of solids slurry. The pH of the slurry was adjusted to 12.0 with 5% by volume NaOH solution. From the slurry, 2 mL was withdrawn into a centrifuge tube and the soluble portion was collected after centrifugation. This was used as the baseline for the Brix measurement.

The slurry was heated with a water bath set to 97° C. and stirred at 100 rpm for 3 hours. The sample was then allowed to cool to room temperature and the mass was determined to be 556.5 g, meaning that 46.67 g of water was lost to evaporation. The slurry was then filtered through a Büchner funnel with filter paper and the filtrate was collected and massed. This first filtrate of Trial 18 was found to have a mass of 252.84 g, a pH of 8.22 and a Brix value of 1.3 (ds=3.29 g). The dry solids were then washed with another 200 g of water and the filtrate was collected and massed. This second filtrate of Trial 18 had a mass of 196.8 g, a pH of 8.59 and a Brix value of 0.3 (ds=0.6 g). This washing procedure was repeated and a third filtrate of Trial 18 was collected having a mass of 205.9 g, a pH of 9.02, and a Brix value of 9. The first and second filtrates of Trial 18 were combined into a single solution with a mass of 444.94 g and the third filtrate was discarded. The combined filtrate of Trial 18 was then poured into a round bottom flask, and rotary evaporated at 55° C. at 120 rpm and 60 mbar until concentrated.

For Trial 19, the same reaction conditions as Trial 18 were used, but the starting material was the enzymatically destarched fiber. Two parallel reactions were run with 6% degree of solids for Trial 19 with an initial pH of 12.0 at 97° C. for 3 hours. The slurry was allowed to cool to room temperature and then filtered through a Büchner funnel with filter paper and the filtrate was collected and massed. This first filtrate of Trial 19 was 493 g and had a pH of 8.65 and a Brix value of 2.1 (ds=10.3 g). The insoluble fiber was then washed with another 200 g of water and a second filtrate was collected having a mass of 408.5 g, a pH of 8.79 and a Brix value of 0.5 (ds=2.0 g). The first and second filtrates of Trial 19 were then combined into a single mass of 901.59 g, poured into a round bottom flask, and rotary evaporated at 55° C. at 120 rpm and 60 mbar until concentrated.

Since larger quantities of NaOH were used in Trials 18 and 19, the crude yield was calculated by the following formula:

Crude yield=(total mass of dried solids in the extract−mass of NaOH)/mass of de-starched fiber

Table 10 shows the crude yield and compositions of Trials 18 and 19.

TABLE 10

| | Trial 18 | Trial 19 |
|---|---|---|
| 5% NaOH | 14.65 | 41.5 |
| solution used (g) | | |
| Starting corn | 24 | 48 |
| fiber (g) | | |
| DS in the extract | 3.89 | 12.3 |
| (g) | | |
| DS in the extract | 3.15 | 10.23 |
| minus NaOH (g) | | |
| Crude yield | 13.2% | 21.3% |
| AX Concentrate- | 3.00% | 10.70% |
| DS % | | |
| AX Concentrate - | 6.0% | 4.7% |
| starch % dsb | | |
| Residual fiber - | 96.84% | 91.37% |
| DS % | | |
| Residual fiber - | 1.2% | 2.2% |
| starch % dsb | | |

A summary of the trials and results of Examples 8-10 is shown in Table 11.

TABLE 11

| Trial | | Initial | Temp. | Reaction | Initial | Crude |
|---|---|---|---|---|---|---|
| No. | Description | pH | (° C.) | time (h) | fiber ds % | yield |
| 11 | De-starched fiber un- | 8.8 | 97 | 3 | 6.4% | 5.6% |
| | milled | | | | | |
| 12 | De-starched fiber un- | 7.5 | 97 | 3 | 6.4% | 5.7% |
| | milled | | | | | |
| 13 | De-starched fiber un- | 5.6 | 97 | 3 | 6.4% | 4.1% |
| | milled | | | | | |
| 14 | De-starched fiber un- | 7.5 | 133 | 1 | 6% | 8.4% |
| | milled | | | | | |
| 15 | De-starched fiber un- | 8.0 | 97 | 3 | 6% | 8.5% |
| | milled | | | | | |
| 16 | De-starched fiber | 8.0 | 97 | 3 | 6% | 12.5% |
| | milled and residual | | | | | |
| | fiber washed with | | | | | |
| | extra water | | | | | |
| 17 | De-starched fiber | 8.0 | 97 | 3 | 10% | 9% |
| | milled and residual | | | | | |
| | fiber washed with | | | | | |
| | extra water | | | | | |
| 18 | Starting fiber was the | 12 | 97 | 3 | 6% | 13.2% |
| | residual fiber after | | | | | |
| | extraction in Trial 16 | | | | | |
| 19 | De-starched fiber | 12 | 97 | 3 | 6% | 21.3% |
| | milled and residual | | | | | |
| | fiber washed with | | | | | |
| | extra water | | | | | |

The extractions carried out in the neutral pH zone (pH 5-8) in Trials 11-16 gave low crude extraction yield (4.1-12.5%), and it is observed that crude yield increases with the increase of initial pH. Extraction at a higher temperature of 133° C. resulted in slightly higher yield than extractions performed at 97° C., but it is still only 8.4%. Among these reactions, a better yield of 12.5% in Trial 16 was achieved when the starting material was ground to powder, and extra washing with water was used to recover more soluble product from the residual fiber after extraction. The degree of solids of starting material also had an impact on the extraction yield. When the starting material level increased to 10% in Trial 17, the extraction yield was reduced to 9% compared to that in Trial 16. Much better yield was achieved when the initial pH was increased to 12 as seen with the crude yield of 13.2% and 21.3% for Trial 18 and Trial 19. The lower extraction yield in Trial 18 was likely due to lower availability of soluble arabinoxylan since the starting material was the residual fiber from Trial 16.

For Trials 11-16, not only was the crude extraction yield low, but the product also contained high percentages of starch and protein. The starch content in the products of Trials 18 and 19 was significantly lower.

### Example 11. Additional Enzyme Destarching and Alkaline Extraction of Arabinoxylan

Wet de-starched fiber (1500 g) from FRONTIA® Enzymatic De-starching treatment, as described in Examples 5-7, was dried in a 40° C. oven for approximately 48 hours and ground to a powder on the Thomas Mill using a 1 mm screen to provide 272 g powder. Approximately 100 g of milled fiber (DSB) was added to a beaker and tap water was added until the total mass was 1004.5 g. The initial pH of the solution was 5.01 and adjusted with NaOH to pH 5.98. The solution was then mixed with 0.066 g of CaCl₂ and placed on a hotplate at 65° C. with a stir bar rotating at 200 rpm. Once the temperature of the solution was at 65° C., 42 μL of alpha-amylase (BAN 480 LS enzyme) was added. A 1 mL aliquot of the solution was then removed with a syringe to measure the Brix value. This was repeated every 20 minutes for 80 minutes resulting in the Brix values of 0.4, 0.5, 0.7, 0.7, and 0.8. The slurry was then filtered through a Buchner funnel and the dry solid was collected and dried.

Extraction of the arabinoxylan (Trial 20) was performed by adding 30 g (dry solid based) of the dried BAN 480LS de-starched fiber into the 1L glass beaker and adding water to approximately 500 g. NaOH pellets (4.0 g) was added to the slurry (NaOH usage level at 0.8 g/100 g slurry), and the beaker was then placed on a hotplate with a stir bar at 97° C., 250 rpm for 1 hour. After the reaction, the slurry was cooled down to the room temperature, and was split into two 250-mL centrifuge bottles. The slurry was centrifuged at 6000 rpm for 15 min. The supernatant was combined (269 g) and the pH was measured to be pH 12. Each pellet in the bottle was re-suspended in 100 g water and the centrifugation was repeated. The combined supernatant (439 g) was adjusted to pH 7 with 1N HCl, and Brix of the solution was 5.3. Therefore, approximately total dry solid of 23.3 g was extracted. The filtrate was then concentrated on a rotary evaporated at 55° C., 60 mbar, and 120 rpm. The mass of the concentrated extract was 130.93 g with a Brix value of 16.8 (22.2 g dry solid recovered). The solid content of this concentrate was also measured on the Computrac to be 17%, similar to the value on the Brix refractometer. The residual fiber after extraction was dried in the oven and the composition was analyzed. The composition of the arabinoxylan extract and the residual fiber are reported in Table 12.

TABLE 12

| | starch | | | | |
|---|---|---|---|---|---|
| Sample Name | content | protein | ash | fat | fiber |
| Trial 20 - Residual | 4.47% | 1.55% | 18.22% | 12.17% | 63.59% |
| Fiber After | | | | | |
| alkaline extraction | | | | | |
| Trial 20 - AX- | 2.59% | 6.55% | 26.85% | 0.69% | 63.33% |
| Concentrate from | | | | | |
| alkaline extraction | | | | | |

The carbohydrate composition of the extract was measured using gas chromatography mass spectroscopy after acid hydrolysis of all polysaccharides to monosaccharides. The carbohydrate composition of the arabinoxylan extract is reported in Table 13.

TABLE 13

| | Trial 20 - AX- |
|---|---|
| | Concentrate |
| | % Composition |
| Arabinose | 40.9 ± 5.1 |
| Xylose | 45.6 ± 6.3 |
| Galactose | 2.4 ± 0.2 |
| Glucose | 1.0 ± 0.1 |

### Example 12. Impact of Crude Extraction Yield by NaOH Level, Reaction Time, and Solid Level of De-Starched Corn Fiber

Arabinoxylan extractions were performed at different NaOH concentrations on the de-starched com fiber after FRONTIA® Fiber wash enzyme treatment, as described in Examples 5-6. The additional treatment with alpha-amylase, as described in Example 11. was not performed. The extraction procedure was similar as that described in Example 11, with variation in parameters the following parameters: NaOH usage level, reaction time, and de-starched corn fiber content. The pH was adjusted to 7.0 after the extraction was complete and the slurry was cooled to room temperature. Centrifugation and pellet washing was performed in the similar procedures as described above to collect the soluble extract. The crude extraction yield was determined as the following:

crude yield=(mass of combined supernatant collected after centrifugation X Brix %−mass of NaOH added)/mass of de-starched corn fiber

The crude extraction yield and various parameters of this example are shown in Table 14.

TABLE 14

| | Trial 21 | Trial 22 | Trial 23 | Trial 24 | Trial 25 |
|---|---|---|---|---|---|
| Reaction | 1 | 2 | 4 | 1 | 1 |
| time (h) | | | | | |
| Temperature | 97 | 97 | 97 | 97 | 97 |
| (° C.) | | | | | |
| DS % of | 6% | 6% | 6% | 10% | 10% |
| destarched corn | | | | | |
| fiber | | | | | |
| DS of | 30 | 30 | 30 | 50 | 50 |
| destarched | | | | | |
| fiber (g) | | | | | |
| Slurry weight | 500 | 500 | 500 | 500 | 500 |
| (g) | | | | | |
| NaOH pellet | 4 | 2 | 2 | 4 | 6.6 |
| (g) | | | | | |
| pH at the end | 12 | 9.56 | 9.6 | 10.99 | 12.3 |
| of the | | | | | |
| reaction | | | | | |
| pH after | 7 | 7 | 7 | 7 | 7 |
| adjustment | | | | | |
| Supernatant | 439 | 320.16 | 466.21 | 367.11 | 423.62 |
| mass (g) | | | | | |
| Supernatant | 5.3 | 4.4 | 3.6 | 7 | 7.6 |
| Brix | | | | | |
| Amount of | 23.267 | 14.1 | 16.8 | 25.7 | 32.2 |
| solid | | | | | |
| extracted (g) | | | | | |
| Crude | 64.2 | 40.3 | 49.3 | 43.4 | 51.2 |
| Extraction | | | | | |
| Yield | | | | | |

### Example 13. Diafiltration to Lower Ash Content

A skid filtration system with UA60 membrane (1 kDa molecular weight cut-off) was flushed with water, washed with 2 L of a pH 2 acid wash solution and 2 L of a pH 11 alkaline wash solution. The device was then flushed twice with water again to clean out the system.

An arabinoxylan extract solution (2778 g), prepared by the methods as described herein, was loaded into the filtration device. Retentate and permeate samples were collected and an extra volume of 1400 ml of water was repeatedly added to dilute the retentate during the nanofiltration. Retentate and permeate samples were collected from this filtration run and another volume of 1400 ml of water was poured into the device. This was repeated for a total six washings and the Brix value for the permeate and retentate samples was measured and recorded. These results are reported in Table 15.

TABLE 15

| | | % ds (by | Permeate | | | | |
|---|---|---|---|---|---|---|---|
| | | Refractive | Volume | Time to | Permeate | | |
| Wash | | Index, | filled | fill | Flow rate | Flux | DS |
| No. | Description | Brix) | (g) | (min) | (g/min) | (L/m²/h) | (g) |
| Control | Water at 75 psi | — | — | — | 450 | 59 | — |
| 1 | AXOS NF DV 0.5 | 0.9 | 1400 | 16.2 | 86 | 11 | 12.6 |
| | perm | | | | | | |
| 1 | AXOS NF DV 0.5 | 4.3 | — | — | — | 0 | — |
| | ret | | | | | | |
| 2 | AXOS NF DV 1 | 0.7 | 1400 | 13.5 | 104 | 14 | 9.8 |
| | perm | | | | | | |
| 2 | AXOS NF DV 1 ret | 3.7 | — | — | — | 0 | — |
| 3 | AXOS NF DV 1.5 | 0.4 | 1400 | 11.3 | 124 | 16 | 5.6 |
| | perm | | | | | | |
| 3 | AXOS NF DV 1.5 | 3.6 | — | — | — | 0 | — |
| | ret | | | | | | |
| 4 | AXOS NF DV 2 | 0.3 | 1400 | 11 | 127 | 17 | 4.2 |
| | perm | | | | | | |
| 4 | AXOS NF DV 2 ret | 3.5 | | | | 0 | |
| 5 | AXOS NF DV 2.5 | 0.2 | 1400 | 11 | 128 | 17 | 2.8 |
| | perm | | | | | | |
| 5 | AXOS NF DV 2.5 | 3.4 | — | — | — | 0 | — |
| | ret | | | | | | |
| 6 | AXOS NF DV 3 | 0.2 | 1400 | 10.6 | 132 | 17 | 2.8 |
| | perm | | | | | | |
| 6 | AXOS NF DV 3 ret | 3.3 | — | — | — | 0 | — |
| | AXOS NF Final | 0.2 | — | — | — | — | — |
| | Perm | | | | | | |
| | AXOS NF Final ret | 4.1 | — | — | — | — | — |

The final retentate was collected and measured to have a mass of 724.06 g and a Brix value of 4.1. The final retentate was then concentrated on a rotary evaporator. The concentrate solution was found to have a mass of 237.13 and a Brix value of 10.0, although extreme foaming occurred during the rotary evaporating process. The recovered mass in the retentate was 23.7 g. The mass input and recollected in the filtering process are shown in Table 16.

TABLE 16

| | |
|---|---|
| total DS in permeate (g) | 37.8 |
| DS in Retentate before rotary evaporation (g) | 29.7 |
| Retentate concentrated (g) | 23.7 |
| total DS in permeate and retentate before rotary evaporation (g) | 67.5 |
| total DS input (g) | 116.7 |
| mass loss in nano-filtration (g) | 49.2 |

The composition before and after the diafiltration was compared are reported in Table 17 and 18.

TABLE 17

| | | | | | Fiber |
|---|---|---|---|---|---|
| | Starch % | Ash % | Protein % | Fat % | content |
| Description | dsb | (dsb) | (dsb) | (dsb) | (calc.) |
| Trial 26 - AX | 5.39% | 28.2% | 8.457% | 0.296% | 57.6% |
| concentrate | | | | | |
| before | | | | | |
| diafiltration | | | | | |
| Trial 26 - AX | 5.63% | 6.9% | 10.60% | 0.52% | 76.4% |
| retentate | | | | | |
| after | | | | | |
| diafiltration | | | | | |

TABLE 18

| | Trial 26 - | Trial 26 - |
|---|---|---|
| | AX concentrate | AX retentate |
| | before | after |
| Total % carbohydrate | diafiltration | diafiltration |
| composition | (dsb) | (dsb) |
| Arabinose | 33.8 ± 6.2 | 36.9 ± 3.4 |
| Xylose | 33.2 ± 4.2 | 39.7 ± 2.4 |
| Galactose | 2.2 ± 0.5 | 2.8 ± 0.4 |
| Glucose | 1.0 ± 0.3 | 0.8 ± 0.2 |
| Ferulic acid (μg/g) | 8523.4 ± 760.9 | 10.4 ± 1.0 |

Additionally, the molecular weight before and after diafiltration was compared, the results of which are reported in Table 19. The diafiltration was able to remove some of the small molecular weight substance that appears in the GPC chromatogram from 2.5 min to 3.00 min retention time, resulting in lower polydispersity, i.e. narrower molecular weight distribution. The molecular weight of the AX polymer was measured to be 150 kDa on the GPC after diafiltration.

TABLE 19

| Sample Name | Mn | Mw | Polydispersity |
|---|---|---|---|
| Trial 26 - AX concentrate | 1094 | 136389 | 124.6 |
| before diafiltration | | | |
| Trial 26 - AX retentate | 4859 | 150587 | 31.1 |
| after diafiltration | | | |

### Example 14. Endo-Xylanase to Lower Viscosity of Arabinoxylan

Endo-xylanase SHEARZYME® (Novozymes) was used to treat an arabinoxylan concentrate. The enzyme was diluted 50-fold before adding to the arabinoxylan. Arabinoxylan concentrate (10 g) was added into each of the 2.4 oz. glass jars, and various amounts of diluted SHEARZYME® was added. No enzyme was added in reaction #1 (Table 19). The reactions were incubated at 45° C. for 1h, followed by 10 min in the 95° C. water bath to deactivate the enzyme. The viscosity of the reaction mixtures were than measured on the rheometer DHR-3 with the parallel plate at 25° C. (LIMS 524000-524002). Table 20 reports the reaction conditions for treating the arabinoxylan.

TABLE 20

| | | | Diluted |
|---|---|---|---|
| | AX concentration | Tap | SHEARZYME ® 2X |
| Reaction # | trial 16 (g) | water (g) | (μl) |
| 1 | 10 | 10 | 0 |
| 2 | 10 | 10 | 10 |
| 3 | 10 | 10 | 50 |

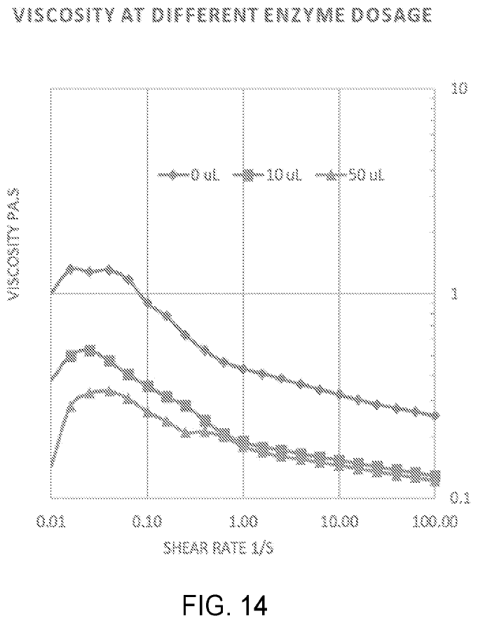

FIG. 14 show the flow curves of each reaction and shows that all the samples show shear thinning behavior. The enzyme treated AX extract showed lower viscosity at all shear rate than the non-treated arabinoxylan.

### Example 15. Production and Purification of Arabinoxylan

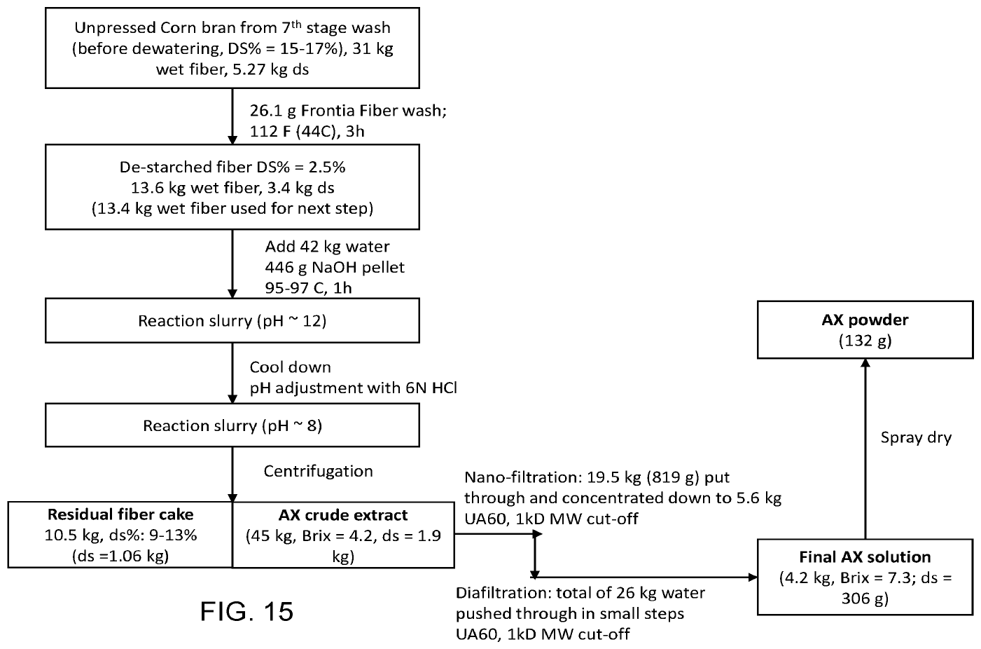

The extraction and purification process of arabinoxylan as described herein was conducted at a larger scale to better understand the process and collect hundreds of grams of sample for physical and compositional characterization. The multi-step process to produce arabinoxylan from corn bran includes a de-starching step with FRONTIA® Fiber wash enzyme, arabinoxylan extraction at alkaline pH and high temperature, removing insoluble fiber through centrifugation, diafiltration to remove ash and finally spray drying to obtain the final product. FIG. 15 provides an overview of this process.

### Destarching

To destarch crude com fiber, a total mass of 31042 g was measured into the Kettle along with an equivalent mass of water (31292 g). The Kettle agitator was then turned on to mix the corn fiber and heat the solution to approximately 112° F. (44.44° C.). Once at temperature, a total mass of 26.0 9 g (0.84 g/kg) of FRONTIA® Fiber wash was added to the solution. The reaction was conducted for 3 hours, at which point it was drained into 5 gallon pails and filtered to remove any liquid present. A total mass of 13686 g of corn fiber was obtained from this procedure.

A mass of approximately 100 g (102.49 g) of this corn fiber sample was placed in the 50° C. oven to dry overnight while the rest was placed in the cold room for future use. The final weight of dried fiber was 27.61 g, which has a residual moisture content of 5.72% on the Computrac. Therefore, the DS % in the wet fiber was calculated to be 25.4% after destarching.

### Extraction

The de-starched wet fiber 13384 g (ds=3346 g) was mixed with 42382 g water and 446 g NaOH (solid). The slurry was heated to 97° C. and maintained at 95-97° C. for 1 h. The slurry was cooled down to room temperature with cooling water and the pH was adjusted to 8.0 with 6N HCl (1372 g HCl solution). The pH change was very slowly initially and dropped faster after pH 10.8.

The slurry was divided into 4 buckets for further processing with a centrifugation to separate the soluble fraction from insoluble. The supernatant was then poured through 2 5um screen to remove any large particles that did not settle well in the centrifuge. The moisture level in the fiber cake was measured on the Computrac, and the solid content in the filtrate was measured on the hand-held refractometer (Brix meter). These results are shown in Table 21.

TABLE 21

| | | | fiber | ds in | % ds in | DS in |
|---|---|---|---|---|---|---|
| Slurry | filtrate | filtrate | cake | filtrate | fiber | filter |
| No. | (g) | Brix | (g) | (g) | cake | cake (g) |
| 1 | 12493 | 4.2 | 2720 | 524.706 | 13% | 353.6 |
| 2 | 12227 | 4.2 | 2833 | 513.534 | 9% | 254.97 |
| 3 | 10929 | 4.2 | 2687 | 459.018 | 9% | 241.83 |
| 4 | 9655 | 4.2 | 2287 | 405.51 | 9% | 205.83 |
| total | 45304 | | 10527 | 1902.77 | | 1056.23 |

The crude extraction yield is calculated following the formula below, which gave a crude yield of 43.5%.

Crude yield=(total mass of DS in the extract—mass of NaOH)/mass of de-starched fiber

To understand how much more soluble product could be recovered from washing the residual fiber cake, 500 g of the fiber cake was washed with 500 g of water, and an extra 5 g of ds was recovered (500 g supernatant with Brix 1.1). Therefore, even with an extra washing step, the crude yield would only be of 47% (105 g of soluble ds would be recovered from 10527 g of fiber cake).

The crude extraction yield for the AX extraction step is lower than what normally achieved at lab scale (see Example 12), possible causes are: loss of materials during the material transfer (e.g., some materials stuck in the pipes from kettle to bucket) and inaccurate solid content measurement (too high) in the de-starched corn fiber, and extraction somehow inefficient in this run (reason unknown).

### Filtration

The skid filtration system (with a UA60 (1 kDa molecular weight cut-off) membrane) was flushed twice with water. Another water washing was then conducted to measure the flux of the filtration device. The device was then washed with 2 L of a pH 2 acid wash solution, 2 L of a pH 11 alkaline wash solution, and then flushed twice with water again to clean the system.

19.5 kg (ds=819 g) of the AX extract was processed by nanofiltration followed by diafiltration (the rest was stored for later use). The nanofiltration concentrated the AX extract to 5.6 kg. Effort was made to concentrate the extract as much as possible before performing the diafiltration to increase the de-salting efficiency. Diafiltration was mostly run at 200 psi with flow rate of 36-40 mL/min measured by the weight filtrate collected in 1 min. The pressure was also increased to 250 psi, and the collected filtrate flow rate was observed to be around 60 g/min. Total amount of 26 kg water was pushed through in small batches (e.g., for every 2 kg filtrate collected, 2 kg fresh water was added to the sample tank). 4200 g retentate was collected, and Brix measured to be 7.3.

The filtering process removed the ash effectively which lowered the ash content from 34.5% to 4.4%.

### Spray-Drying

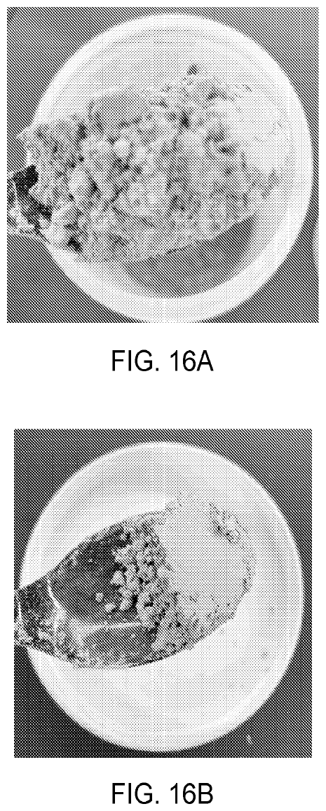

The filtered AX product was then spray-dried. The spray-dryer in the pilot lab was used instead. The inlet air temperature was set to 220° while the outlet air temperature was kept within 85° to 95°. The spray drier recovered approximately 132 g of powdered AX from 4.1 kg solution at Brix of 7.3 (299 g ds), which gave the recovery yield of 44.1%. A second darker colored AX flakes was recovered from the wall of the chamber and collected into a separate bag (30 g). FIG. 16A and 16B show pictures of the powdered AX and darker color AX collected, respectively. A 20g sample of the AX powder was placed in a glass vial for composition analysis and the remaining powder was stored for future use.

### Composition Analysis

Samples were collected at different points during the process described above and their composition was evaluated. All samples were dried in the oven at 50° C. and milled in the coffee grinder before analysis, except for the final retentate from diafiltration that was spray dried. Table 22 reports these compositions. Additionally, the carbohydrate composition of the arabinoxylan product was evaluated and compared to Trail 26 as discussed in Example 13. These results are reporting in Table 23.

TABLE 22

| Sample Information | starch | protein | fat | ash | moisture |
|---|---|---|---|---|---|
| destarched fiber | 3.43% | 6.80% | 6.10% | 0.43% | 8.36% |
| residual fiber after | 4.66% | 2.44% | 4.81% | 9.84% | 3.59% |
| Rotabel (dry) | | | | | |
| residual fiber after | 4.37% | 2.32% | 4.98% | 9.40% | 5.86% |
| floor centrifuge (dry) | | | | | |
| residual fiber after | 4.74% | 1.44% | 5.29% | 5.08% | 3.45% |
| extra washing (dry) | | | | | |
| AX extract | 0 | 7.07% | | 34.60% | 5.35% |
| concentrate (dry) | | | | | |
| retentate from | 0 | 10.30% | 1.17% | 4.43% | 5.39% |
| diafiltration (dry) | | | | | |

TABLE 23

| | Trial 26 - AX | Trial 26 - AX | |
|---|---|---|---|
| | concentrate | retentate | Retentate |
| | before | after | from |
| | diafiltration | diafiltration | diafiltration |
| | (dsb) | (dsb) | (dry) |
| Arabinose | 33.8 ± 6.2 | 36.9 ± 3.4 | 34.3 ± 0.9 |
| Xylose | 33.2 ± 4.2 | 39.7 ± 2.4 | 37.4 ± 1.0 |
| Galactose | 2.2 ± 0.5 | 2.8 ± 0.4 | 9.7 ± 0.7 |
| Glucose | 1.0 ± 0.3 | <1 | <1 |
| Uronic acid | 4.5 ± 0.2 | 8.2 ± 0.3 | 6.3 ± 0.3 |
| Ferulic Acid μg/g | 8523.4 ± 760.9 | 10.4 ± 1.0 | 940.8 ± 92.7 |

### Viscosity

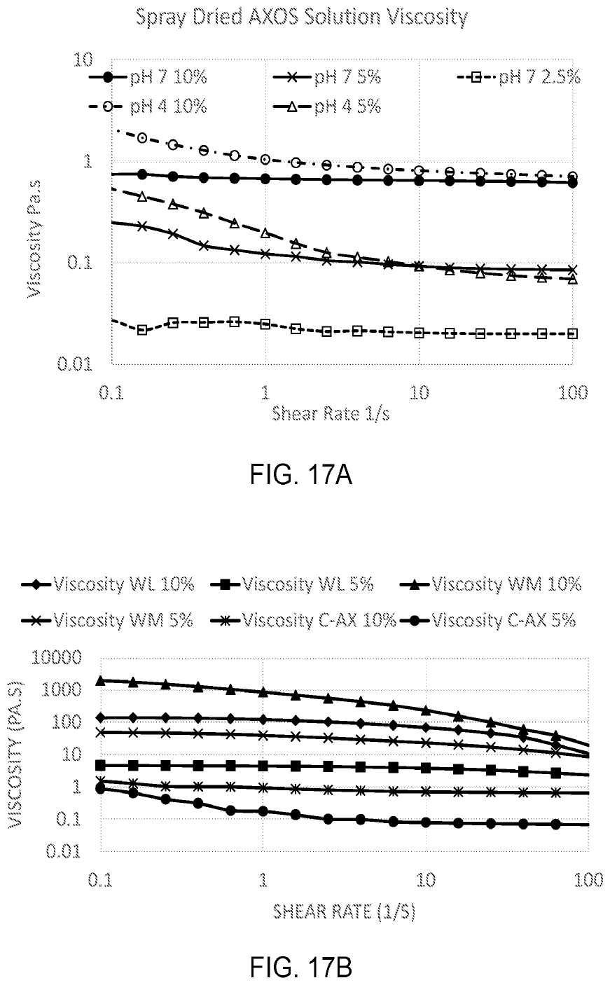

Spray-dried AX (10 g), as prepared above, was dispersed in DI water to make 10% ds solution, and the pH was 7.2. The sample was serial diluted to 5% and 2.5%. 40 g of the 10% solution at pH 7.2 was pH adjusted to 4.0 (0.48 g 2N HCl used). The 5% solution at pH 7.2 was similarly adjusted to a pH of 4. The viscosity sweep at different shear rate was performed. The viscosity curves for these experiments are reported in FIG. 17A and 17B. Shear-thinning at pH 4.0 was observed at 10% and 5%. Without being bound by theory, it is hypothesized that this is due to protein particles being present.

Similar viscosity experiment was performed previously with wheat arabinoxylan samples obtained from Megazyme (WM: wheat AX medium viscosity; WL: wheat AX low viscosity). Additionally, the AX product of Trail 26 (retentate after diafiltration) was also measured. The Trail 26 product showed higher viscosity at the same shear rate than the spray-dried AX product in this study (e.g., 0.92 Pa·s vs 0.67 Pa·s).

### Appearance

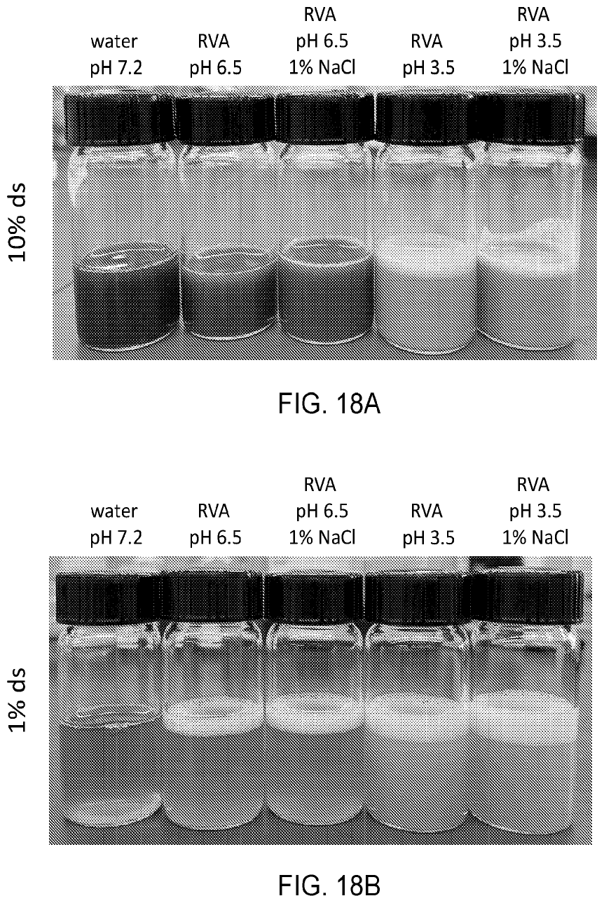

The spray-dried AX was dissolved in water and in different buffers to 10% ds. After a homogenous suspension was formed, 1 g of the 10% suspension was diluted in the same buffer to 1% ds. The look of these different solutions/suspensions is in FIG. 18A and 18B, respectively. In these figures, from left to right the buffers are as follows: In water as-is (pH 7.2), RVA buffer at pH 6.5, RVA buffer pH 6.5 with 1% NaCl, RVA buffer pH 3.5, RVA buffer pH 3.5 with 1% NaCl. The AX product obtained is with color and the solubility is pH dependent. At lower pH, the solution becomes cloudy.

### Example 16. Production of Medium Viscosity Arabinoxylan by Enzymatic Treatment

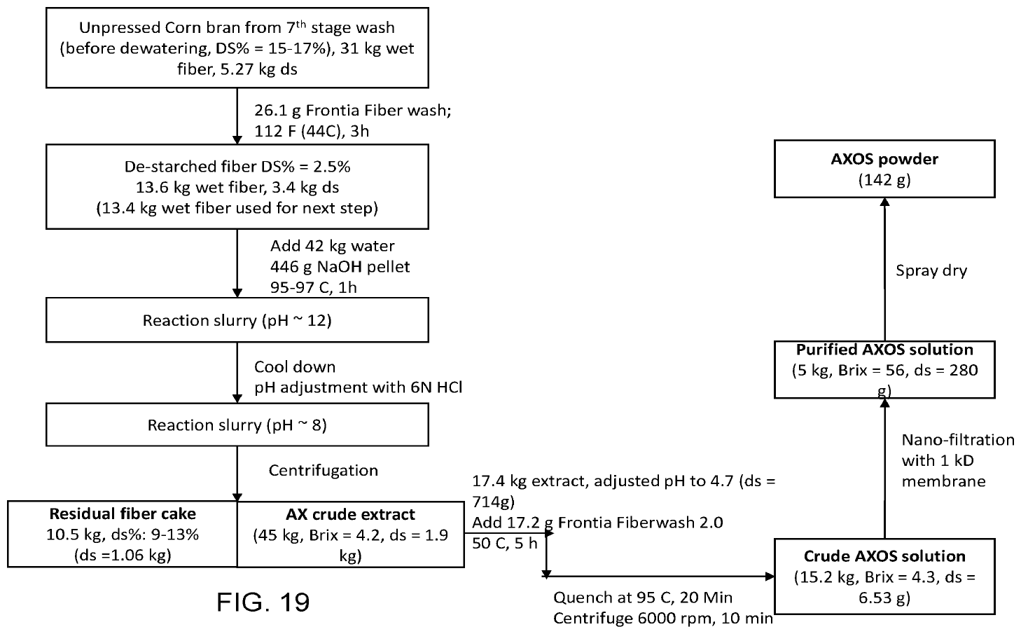

The arabinoxylan product produced in Example 15 was further treated to modify the viscosity of the final product. FIG. 19 outlines this process. The process produced an AX product with reduced viscosity and molecular weight. The solubility and color of this product is better than that of Example 15 and is pH dependent. The nanofiltration step significantly reduced the salt content in the product. There is some material loss in the spray-drying process, but the relative percentage of loss would be minimized when the production scale increases.

### Hydrolyzing with FRONTIA® Fiber Wash 2.0

An AX extract 17.4 kg with Brix of 4.2 (DS=714 g) from Example 15 was added to the 10-Gal kettle, pH was adjusted to 4.7 using 2N HCl, and the temperature was set to 50° C. FRONTIA® Fiber wash 2.0 (17.4 g) was added to the kettle. The reaction mixture was stirred for 5 h, the temperature was raised to 95° C. and held for 20 min to deactivate the enzyme. The mixture was then cooled down to room temperature and centrifuged at 6000 rpm for 10 min in multiple batches to precipitate the insoluble material. The supernatant was combined (15.2 kg, Brix of 4.3, DS=653 g) and subjected to nanofiltration, as described below.

### Filtration

The skid filtration (with a UA60 (1 kDa molecular weight cut-off) membrane) system was flushed twice with water. The device was then washed with 2 L of a pH 2 acid wash solution, 2 L of a pH 11 alkaline wash solution, and then flushed twice with water again to clean the system.

15.2 kg (ds=652 g) of the AXOS solution was processed by diafiltration. It was first concentrated to 3.2 kg via nanofiltration and 11.8 kg permeate was collected. Effort was made to concentrate the extract as much as possible before performing the diafiltration to increase the de-salting efficiency. For diafiltration, 1-2 kg of water was added when every 1 kg of permeate was collected. A total of 13 kg water was added in the process, and 5 kg of concentrate with Brix of 5.6 was collected for spray-drying, as described below.

### Spray-Drying

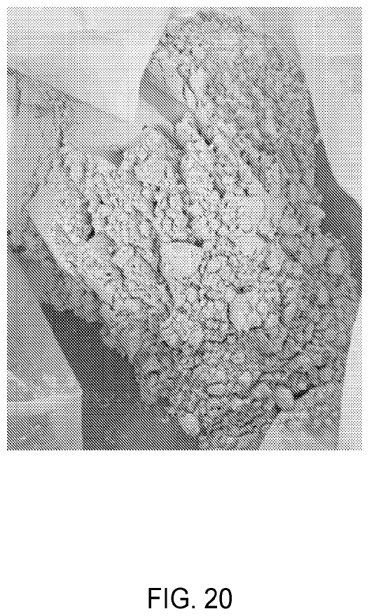

For spray dying, the inlet air temperature was set to 220° while the outlet air temperature was kept within 85° to 95°. The spray drier recovered approximately 140 g of powdered AXOS from 5 kg solution at Brix of 5.6 (280g ds), which gave the recovery yield of 50%. A picture of the resulting spray-dried product is shown in FIG. 20.

### Composition Analysis

Samples were collected at different points during the process described above and their composition was evaluated. All samples were dried in the oven at 50° C. and milled in the coffee grinder before analysis, except for the final retentate from diafiltration that was spray dried. Table 24 reports these compositions.

TABLE 24

| Sample Information | starch | protein | fat | ash | moisture |
|---|---|---|---|---|---|
| AX extract | 0 | 7.07% | Low | 34.60% | 5.35% |
| concentrate (dry) | | | | | |
| Spray dried AX | 0 | 10.30% | 1.17% | 4.43% | 5.39% |
| from Example 15 | | | | | |
| AXOS from Example 16 | 0 | 7.70% | 0.33% | 4.68% | 4.50% |

The AXOS recovery yield from the enzymatic conversion of AX to AXOS, followed by nanofiltration can be calculated as the following:

DS (g) recovered from nanofiltration*(1-ash %-protein%)/DS (g) before enzyme conversion*(1-ash %-protein%)=280*(1-4.68%-7.7%)/714*(1-34.6%-7.7%)=59%

Some mass loss occurred in the transfer of materials from the kettle to the bucket, and to and from the centrifuge bottles (e.g., 714-652=58 g solid lost after the centrifugation step. If assuming same composition in the lost material as in the AX extract, then 33 g AX was lost.

The carbohydrate composition analysis of the AXOS product was also conducted. Arabinose, xylose, galactose and glucose were quantified on GC before and after the acid hydrolysis. Without hydrolysis, no sugar monomers were detected. Table 25 reports these results.

TABLE 25

| Arabinose (%) | Xylose (%) | Galactose (%) | Glucose (%) |
|---|---|---|---|
| 25.4 ± 7.0 | 28.0 ± 6.4 | 3.3 ± 0.1 | 2.1 ± 0.1 |

### Molecular Weight and Rheology

Molecular weight of the arabinoxylan product prepared above was measured on GPC along with a comparative soluble arabinoxylan product and the arabinoxylan prepared previously before enzymatic hydrolysis (e.g., Example 15). The comparative soluble arabinoxylan product is a soluble corn fiber product that has a maximum moisture content of 9 wt. %, a maximum ash content of 6 wt. %, dietary fiber in the range of 74-90 wt. %, and a color value, L, of 30.00. The molecular weight of arabinoxylan prepared in Example 15 without enzymatic hydrolysis is 162431 Da, slightly higher than the comparative soluble arabinoxylan molecular weight of 147613 Da. After enzyme hydrolysis in the Example 16, the product molecular weight was reduced to 31339 Da. These results are shown in Table 26.

TABLE 26

| Sample Information | Mn | Mw | Polydispersity |
|---|---|---|---|
| Comparative soluble | 13079 | 147613 | 11.3 |
| arabinoxylan | | | |
| Corn bran arabinoxylan | 13298 | 162431 | 12.2 |
| prepared in Example | | | |
| 15 | | | |
| Corn bran arabinoxylan | 3785 | 31339 | 8.3 |
| prepared in Example | | | |
| 16 | | | |
| GPC Reference | 1190 | 2023 | 1.7 |

### Viscosity

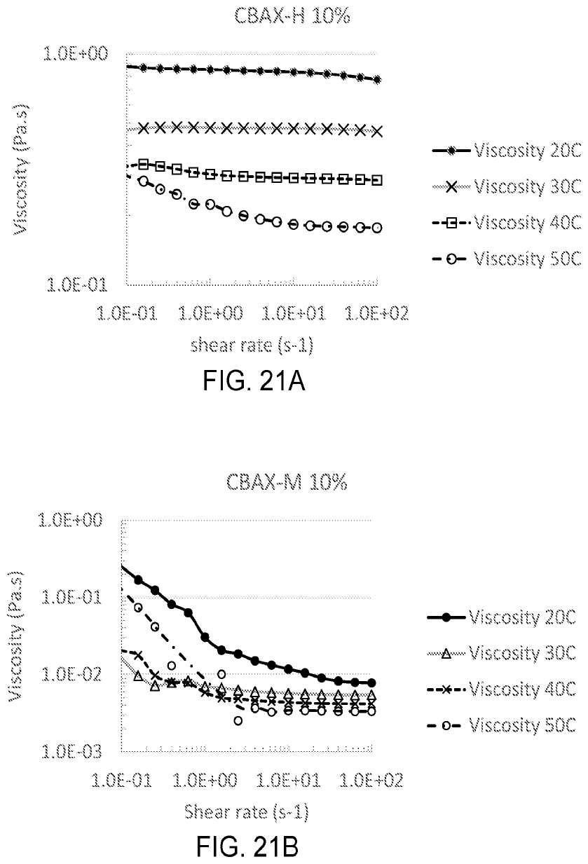

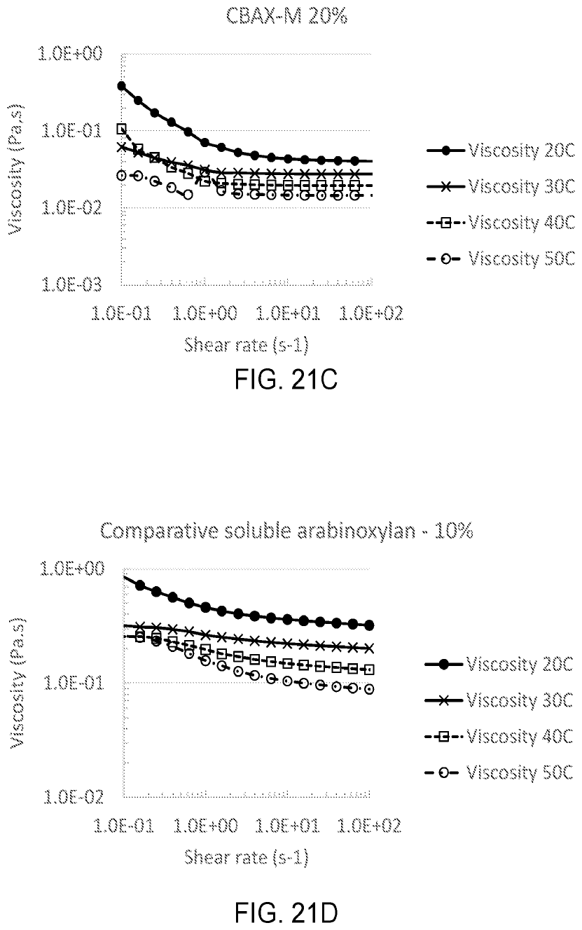

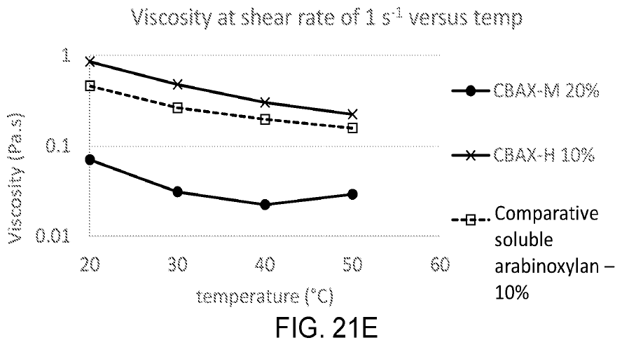

The viscosity of corn arabinoxylan prepared in Example 15 (CBAX-H, LIMS 525113) and the enzymatic hydrolyzed arabinoxylan prepared in as described above in Example 16 (CBAX-M, LIMS 527140) were measured at different temperature from 20° C. to 50° C., and shear rate from 0.1 to 100 s⁻¹. The comparative arabinoxylan product from was also measured as reference. The viscosity curves of these measurements are reported in FIG. 21A-21E. As expected, for all samples viscosity decreased with the increase of temperature.

### Solubility and Color

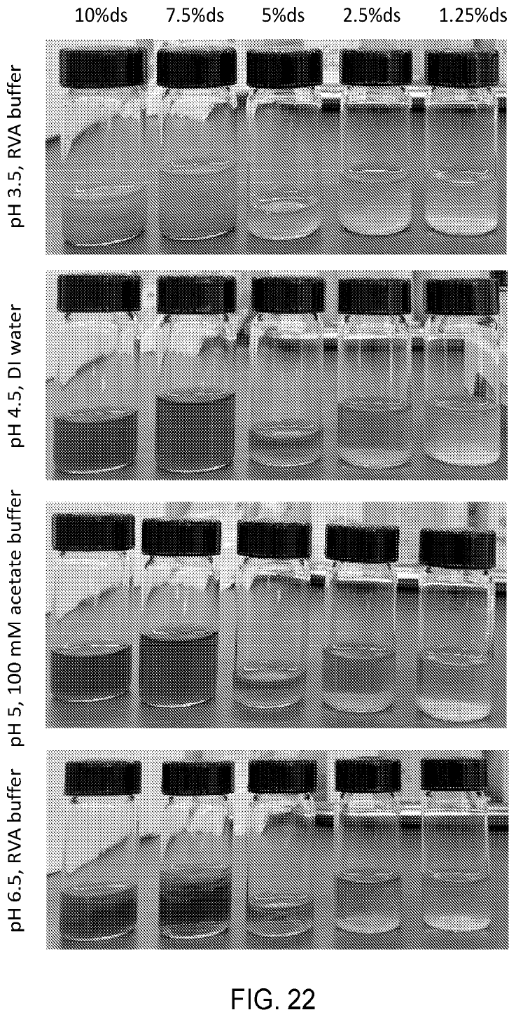

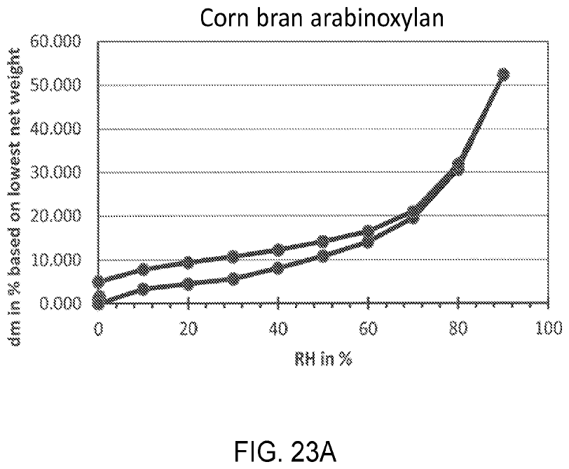

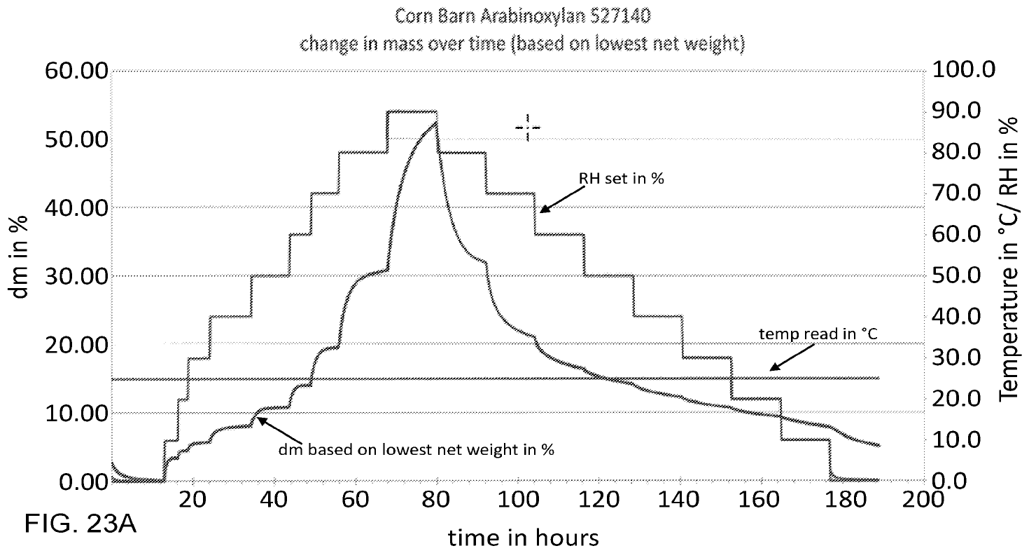

The AXOS product as prepared in Example 16 has brown colors when dissolved in buffers, and the intensity of color and solubility is pH dependent, as shown in FIG. 22. In FIG. 22, from left to right: 10% ds, 7.5% ds, 5% ds, 2.5% ds and 1.25% ds; From top to bottom: in pH 3.5 RVA buffer; in DI water (pH 4.5); in 100 mM acetate buffer pH 5.0; in pH6.5 RVA buffer. The arabinoxylan product of Example 15 has lower solubility at all pHs compared to that of Example 16. Both products showed increased solubility and color intensity when pH increases.

### Moisture Sorption Study

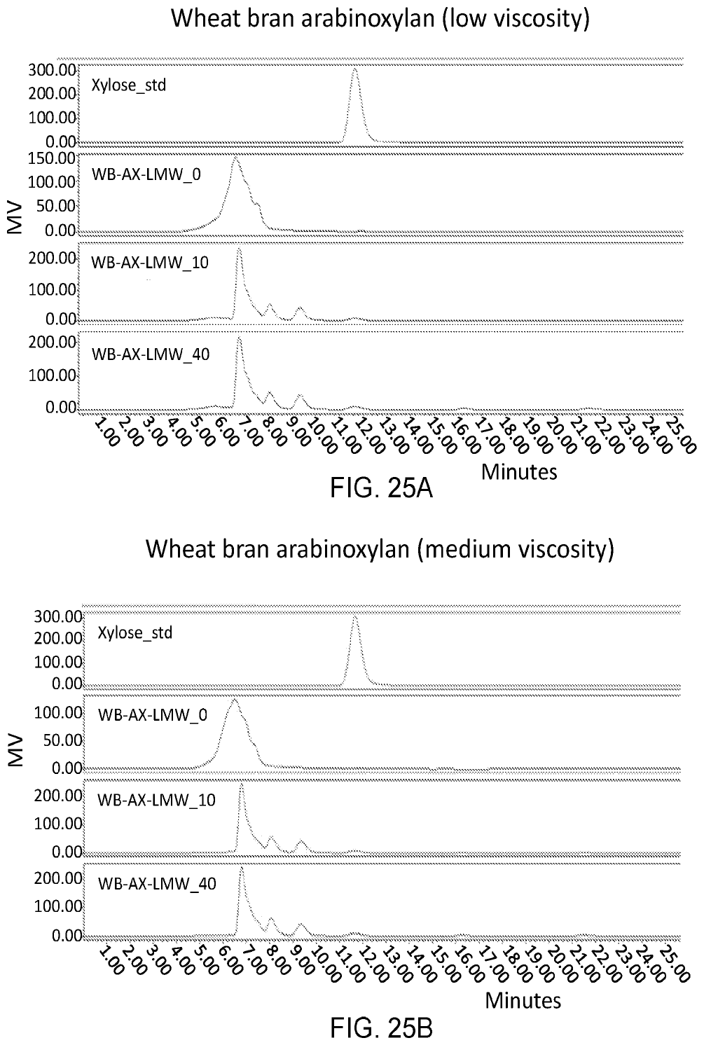

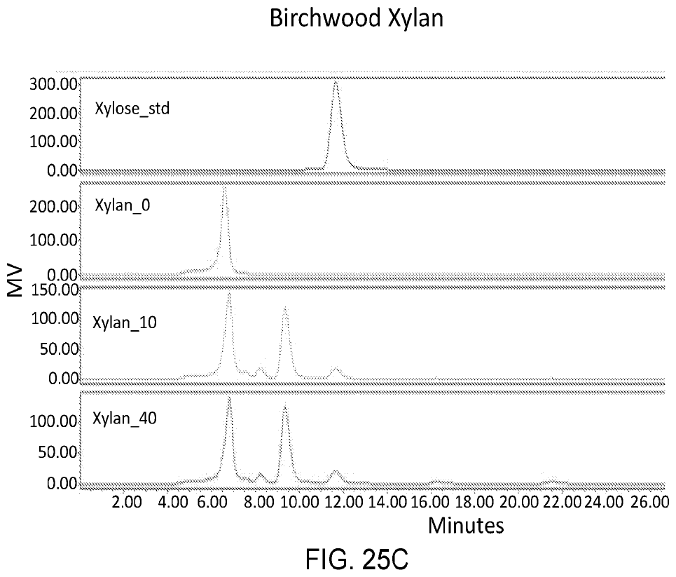

Water sorption data are collected for the AXOS product using dynamic vapor sorption (DVS) with humidity range 0-90% at 25° C. Deliquescence started at 80% humidity for the AXOS product described above. These results are shown in Table 27 and FIG. 25A and 25B.

TABLE 27

| Sorption | Sorption | Desorption | Desorption |
|---|---|---|---|
| Relative | Moisture | Relative | Moisture |
| Humidity (%) | Content (%) | Humidity (%) | Content (%) |
| 0.1 | 0.0116 | 0.1 | 5.0982 |
| 10.0 | 3.3963 | 10.0 | 7.8813 |
| 20.0 | 4.5136 | 20.0 | 9.3788 |
| 30.0 | 5.6965 | 30.0 | 10.7089 |
| 40.0 | 8.0889 | 40.0 | 12.2604 |
| 50.0 | 10.8159 | 50.0 | 14.1740 |
| 60.0 | 14.0353 | 60.0 | 16.4097 |
| 70.0 | 19.5327 | 70.0 | 20.9889 |
| 80.1 | 30.7044 | 80.1 | 31.8853 |
| 90.1 | 52.3987 | N/A | N/A |

### Example 17. Evaluation of Hydrolysis of Arabinoxylan by Different Xylanase

### Endoxylanase Test on Various Xylan Substrates

After extraction, an arabinoxylan products, as described above, was treated with an endoxylanase by following the below procedure:
1. Take ˜140 g AX extract before nano-filtration (the DS % is 4.2), and adjust pH to 5.0;
2. Divide into 3 jars with 40 g in each;
3. Into the 3 jars add SHEARZYMER in the following amounts: 0, 10 μl, 40 μl
4. Incubate at 50° C. overnight;
5. Quench the reaction by increasing the temperature to 90° C. and cook for 10 min

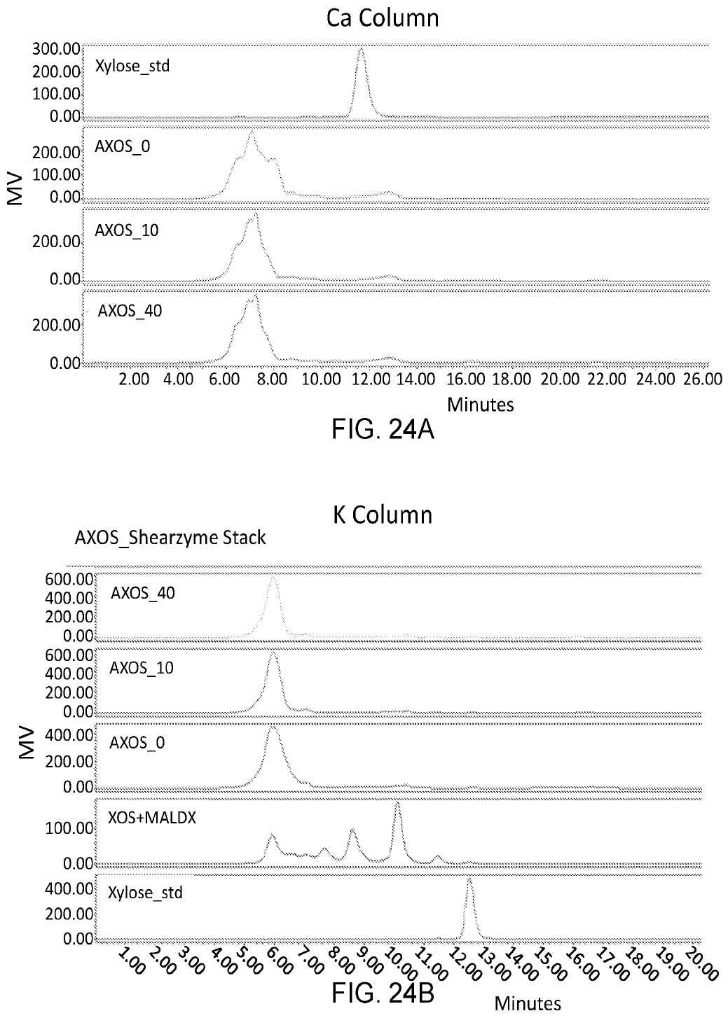

The samples where then characterized with HPLC on both an Aminex HPX-87C Ca column (@85 C, 0.6 ml/min water, RI) and an Aminex HPX-87K column (@85 C, 0.6 ml/min water, RI). Before characterization on the HPLC on the Ca column, the samples were subjected to a resin treatment to remove excess ash and a 0.45 μm filtration. Before characterization on the HPLC on the K column, 5 mL of each sample and filtered through the 3 kDa molecular weight cut-off centrifugal filter and dilute the retentate was diluted with aim to reduce ash load. It was observed that the “emulsion” was broken in the concentrated samples with SHEARZYME® treatment, but not for the non-treated sample. The resulting chromatograms are reporting in FIG. 24A and 24B.

For comparison, the same endoxylanase treatment was conducted on two wheat arabinoxylan with medium and low viscosity (Megazyme) and birch wood xylan (Sigma) and characterized on the HPLC with a Ca column (@85 C, 0.6 ml/min water, RI). All three substrates (at 1% ds) were degraded to a much higher extent than the extracted arabinoxylan from com (FIG. 25A, 25B, 25C). The result indicates that the higher arabinose branching on xylan poses severe steric hindrance for the endo-xylanase to act.

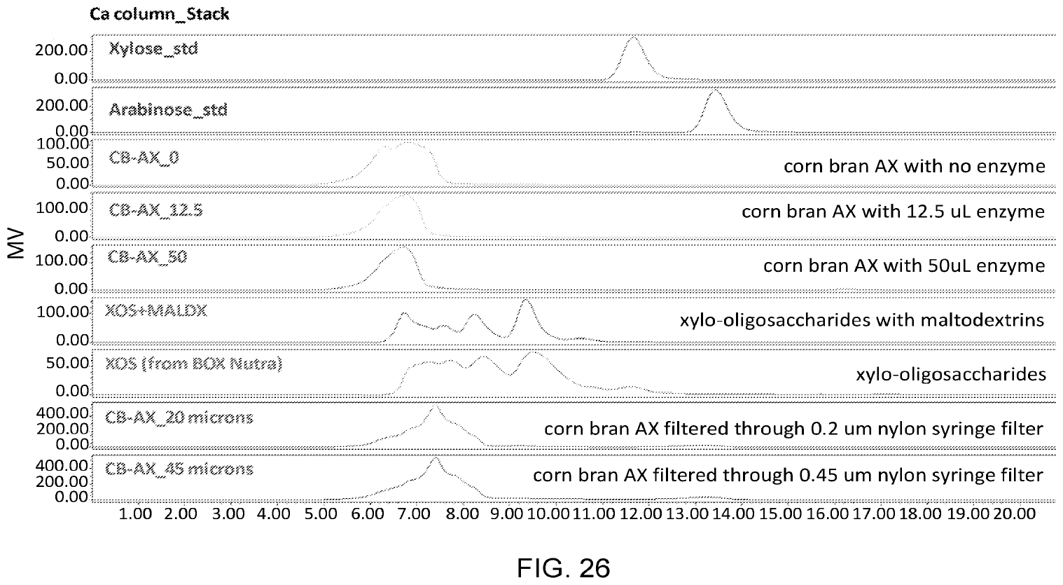

To evaluate if a purified corn bran AX would be more prone to hydrolyzing by SHEARZYMER, the spray-dried AX powder was dissolved at 1% ds in 10 mM acetate buffer at pH 5.0 with 5 mL total volume. Various doses of SHEARZYME® was added (0 μL, 12.5 μL, and 50 μL) and the reactions were performed at 50° C. for overnight. The resulting chromatograms are shown in FIG. 26. Once again, very little shift was observed on the HPLC chromatogram (Ca Column@85 C, 0.6 ml/min water, RI.), indicating that the high salt content in the AX extract did not cause the inefficiency of the enzyme.

### Other Enzymatic Treatment on Various Xylan Substrates

Enzyme C was obtained from Weiss Biotech, which is a mixture of endo-xylanase and glucanase. FRONTIA® Fiber Wash 2.0, provided by Novozymes, consisting an endo-xylanase and cellulase.

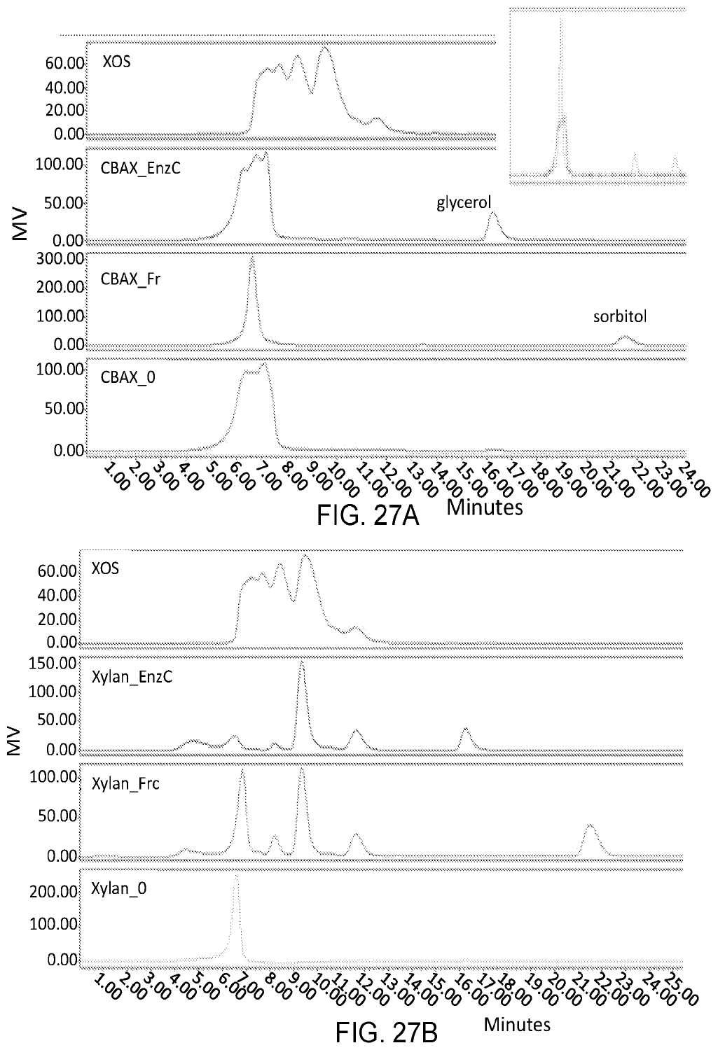

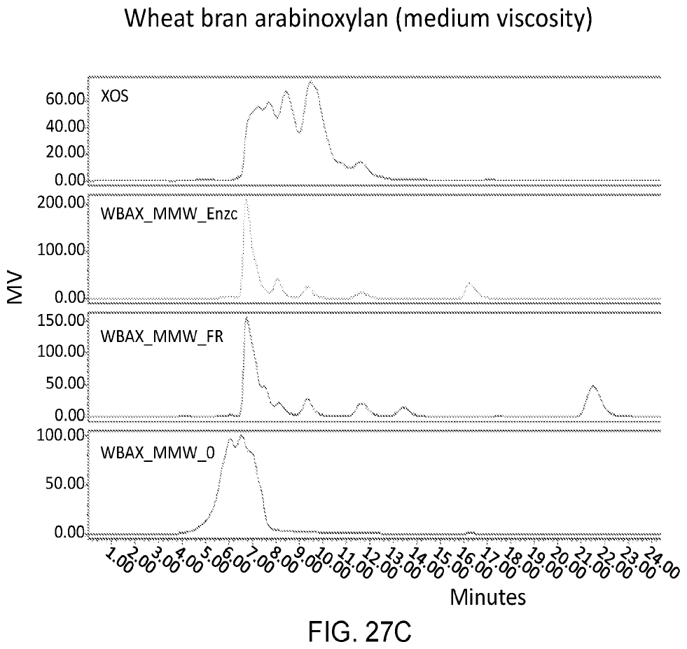

To perform the enzyme treatment, 10 μl of enzyme was added to 5 ml of a xylan or AX solution at 1% DS in 10 mM acetate buffer at pH 5.0. The reactions were conducted at 50° C. overnight, and enzyme activity was quenched by heating the samples at 95° C. for 10 min. For comparison, the same endoxylanase treatment was conducted on a wheat arabinoxylan with medium viscosity (Megazyme) and birch wood xylan (Sigma). The resulting products were characterized via HPLC (Ca column@85 C, 0.6 ml/min water, RI), the results of which are shown in FIGS. 27A, 27B, and 27C.

FRONTIA® Fiber wash 2.0 was observed to be more active than Enzyme C on all three substrates tested: spray-dried corn bran AX (FIG. 27A), birch wood xylan (FIG. 27B) and wheat bran AX (FIG. 26C). Glycerol (Retention time 16.5 min) and sorbitol (retention time 21.6 min) peaks are detected in the Enzyme C and FRONTIA® 2.0 samples respectively, which are the enzyme stabilizers used in the enzyme formulation.

### GPC Analysis of Corn Bran AX Before and After Enzyme Treatment

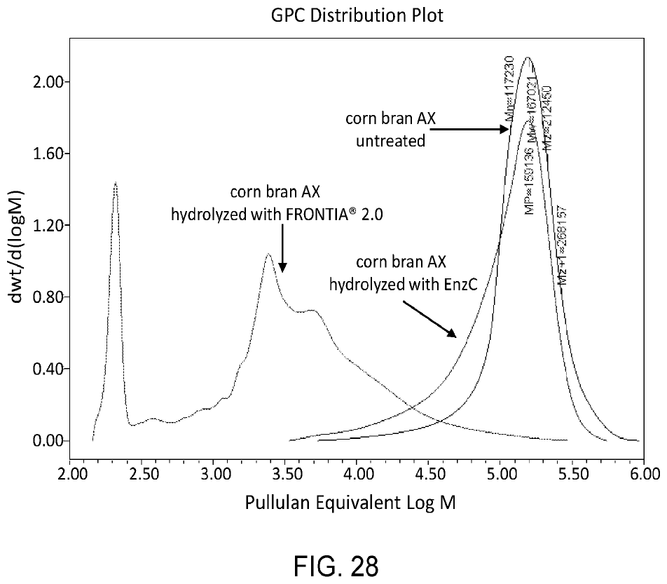

AX samples treated with Enzyme C and FRONTIA® Fiber wash 2.0, as described above, were submitted for molecular weight analysis on GPC. The results are report in Table 28 below and in FIG. 28.

TABLE 28

| | | Mw - AVG | |
|---|---|---|---|
| Description | Mn - AVG (Da) | (Da) | p/d |
| CBAX-untreated | 115,905 | 166,832 | 1.44 |
| CBAX-EnzC | 71,170 | 130,068 | 1.83 |
| CBAX-FRONTIA ® | 2526 | 9080 | 3.6 |
| 2.0 | | | |

Consistent with the results observed on HPLC Ca-column, FRONTIA® Fiber Wash 2.0 was able to reduce the molecular weight much more significantly than Enzyme C. The peak with M.W. around 200 (LogM 2.3) is attributed to sorbitol, which is introduced from the enzyme formula, and it's excluded in the MW calculation (FIG. 28).

### Time Course of Corn Bran AX (Crude Extract) Hydrolysis by FRONTIA® Fiber Wash 2.0

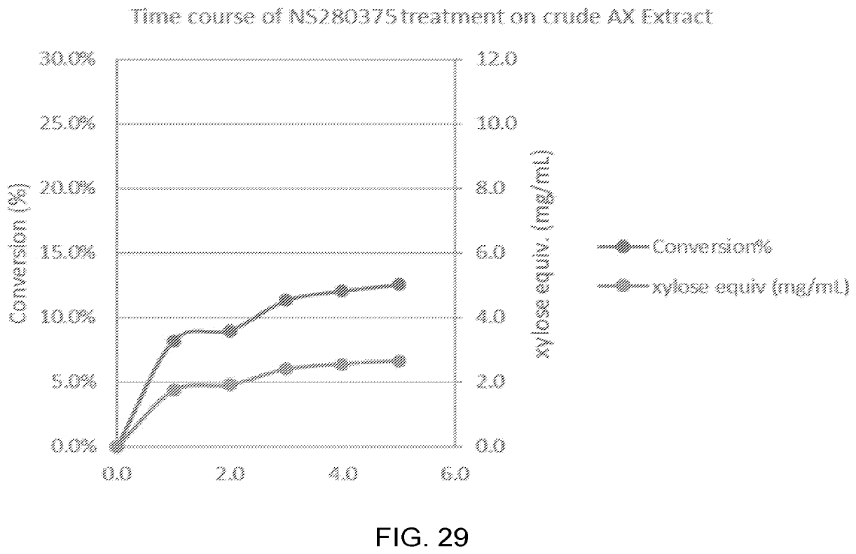

A volume of 5 mL of AX extract (DS %=4.2%, and carbohydrate content is 52%, initial substrate concentration 2.08%; 20.8 mg/mL) was adjusted to pH 5.0 and was pipetted into a 15 mL centrifuge tube. The solution was then placed in the thermomixer and heated at 50° C. and 300 rpm. A sample for baseline was collected by pipetting 50 μl of the AX solution into an Eppendorf tube with 750 μl DNSA reagent. 10 μl of FRONTIA® Fiber Wash 2.0 was added. At time zero (immediately after enzyme addition), 1 h, 2 h, 3 h, 4 h and 5 h, an aliquot of 50 μl or 25 μl sample was transferred to Eppendorf tubes containing 750 μl DNSA reagent. Into each tube, 450 μl or 475 μl water was added to bring the volume to 1.25 mL. The samples were then heated at 98° C. for 15 min, and 250 μl Rochelle's Salt was added subsequently in each tube. Absorption at 540 nm was recorded, and the xylose equivalent was calculated using a standard curve. The results are reports in Table 29 and FIG. 29.

TABLE 29

| | | xylose | | | |
|---|---|---|---|---|---|
| | | equiv | xylose | Net | |
| | Absorbance | amount | concentration | increase | Conversion |
| Time | at 540 | (μg) | (mg/mL) | (mg/mL) | % |
| 0 | 0.155 | 66.9 | 1.338 | 0.000 | 0.0% |
| 1 | 0.971 | 153.7 | 3.074 | 1.736 | 8.3% |
| 2 | 1.05 | 162.1 | 3.242 | 1.904 | 9.2% |
| 3 | 0.404 | 93.4 | 3.736 | 2.398 | 11.7% |
| 4 | 0.441 | 97.3 | 3.893 | 2.555 | 12.3% |
| 5 | 0.465 | 99.9 | 3.995 | 2.657 | 12.8% |

A second reaction was run with double the amount of enzyme used, the results of which are reported in Table 30.

TABLE 30

| | Abs at | | | net | |
|---|---|---|---|---|---|
| | 540 | xylose | xylose | increase | Conversion |
| Time (h) | nm | (μg) | (mg/mL) | (mg/ml) | % |
| Baseline | 0.151 | 56.95652174 | 1.14 | 0.00 | 0.0% |
| 0 | 0.15 | 56.84782609 | 1.14 | 0.00 | 0.0% |
| 1 | 0.998 | 149.0217391 | 2.98 | 1.84 | 8.9% |
| 2 | 1.21 | 172.0652174 | 3.44 | 2.30 | 11.1% |
| 3 | 0.401 | 84.13043478 | 3.37 | 2.23 | 10.7% |
| 4 | 0.372 | 80.97826087 | 3.24 | 2.10 | 10.1% |
| 5 | 0.365 | 80.2173913 | 3.21 | 2.07 | 9.9% |

No improvement in the degree of AX hydrolysis was observed, rather a slight decrease was observed, which could be due to operation errors. The conversion reached to the maximum at 2 h, faster than the previous reaction at lower enzyme concentration (3-4 h was needed to reach the max conversion).

Therefore, it seems that the max conversion (hydrolysis) of AX extract is between 10.7%-12.8%, and more enzyme was not able to push the conversion higher.

### Time Course of Corn Bran AX (Spray-Dried) Hydrolysis by FRONTIA® Fiber Wash 2.0

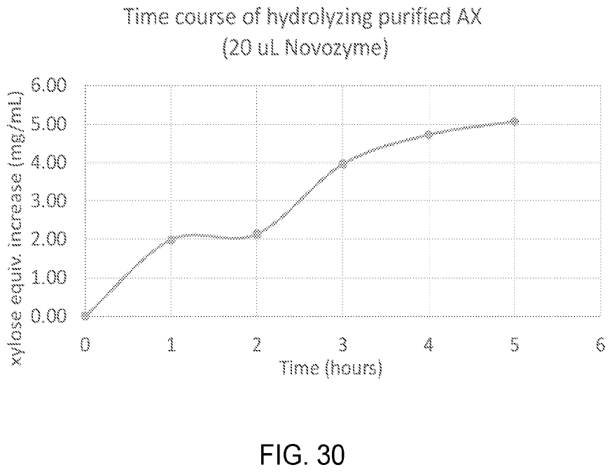

Spray-dried AX was dissolved in 5 ml of a 10 mM acetate buffer at pH 5.0 at 5% ds (substrate concentration 5%, 50 mg/mL) in a 15 mL centrifuge tube, and the tube was incubated at 50° C. Thermomixer for 10 min. 25 μl sample was drawn to quench in the DNSA reagent as the baseline, and 20 μl of enzyme was added to the rest of the sample. At time 0 h, 1 h, 2 h, 3 h, 4 h, and 5 h, 25 μl sample was drawn to quench in the DNSA reagent. The assay was performed as described above. The results are reported in Table 31 and FIG. 30. At 5 h, the degree of hydrolysis reached close to the maximum achieved previously with the AX crude extract, but it would be good to follow up with additional experiment including longer reaction time.

TABLE 31

| | | | | xylose | |
|---|---|---|---|---|---|
| | | xylose | xylose net | concentration | |
| | | equivalent | increase | increase | conversion |
| Time | Abs at 540 nm | (μg) | (μg) | (mg/mL) | (%) |
| Baseline | 0.142 | 57.64 | 0.00 | 0.00 | 0.0% |
| 0 | 0.145 | 57.97 | 0.33 | 0.01 | 0.0% |
| 1 | 0.6 | 107.42 | 49.78 | 1.99 | 4.0% |
| 2 | 0.634 | 111.12 | 53.48 | 2.14 | 4.3% |
| 3 | 1.052 | 156.55 | 98.91 | 3.96 | 7.9% |
| 4 | 1.227 | 175.58 | 117.93 | 4.72 | 9.4% |
| 5 | 1.307 | 184.27 | 126.63 | 5.07 | 10.1% |

### Impact of FRONTIA® 2.0 Treatment on the Viscosity, Protein Content and Appearance of Corn Bran AX

A 10% DS AXOS solution was prepared and treated with the enzyme to determine the impact of enzyme treatment on viscosity. Approximately 10 g of spray dried AXOS powder was measured into an 8 oz. glass jar and diluted to a total mass of 100 g with DI water. The pH of the AXOS solution was then adjusted dropwise to a pH of 5.

To understand if the small molecules (mostly sorbitol) in the enzyme formulation affect the viscosity measurement, a fractionation process was performed on the enzyme. Briefly, 1 mL of FRONTIA® 2.0 enzyme was pipetted into a 10 kDa Millipore filtration unit and centrifuged at 4000 rpm for 10 minutes. The resulting filtrate was labeled as Enz10Kd.

Six 15 mL centrifuge tubes were prepared and 15 mL of 10% DS AXOS solution was pipetted into each centrifuge tube. The first tube was treated with 60 μl of water and labeled Blank, the second tube was treated with 60 μl of Enz10Kd and labeled Enz, and the remaining 4 tubes were treated with 60 μl of FRONTIA® 2.0 and labeled T2, T4, T5, and T22 respectively.

The samples were then placed into a thermomixer and heated at 50° C. and 300 rpm. After 2 hours, the T2 sample was removed and quenched by placing it into a 95° C. water bath for 10 minutes. After 4 hours, the T4 sample was removed and quenched by placing it into a 95° C. water bath for 10 minutes. After 5 hours, the Blank, Enz, and T5 sample was removed and quenched by placing it into a 95° C. water bath for 10 minutes. The T22 sample was left overnight and removed the following morning after 22 hours and quenched by placing it into a 95° C. water bath for 10 minutes.

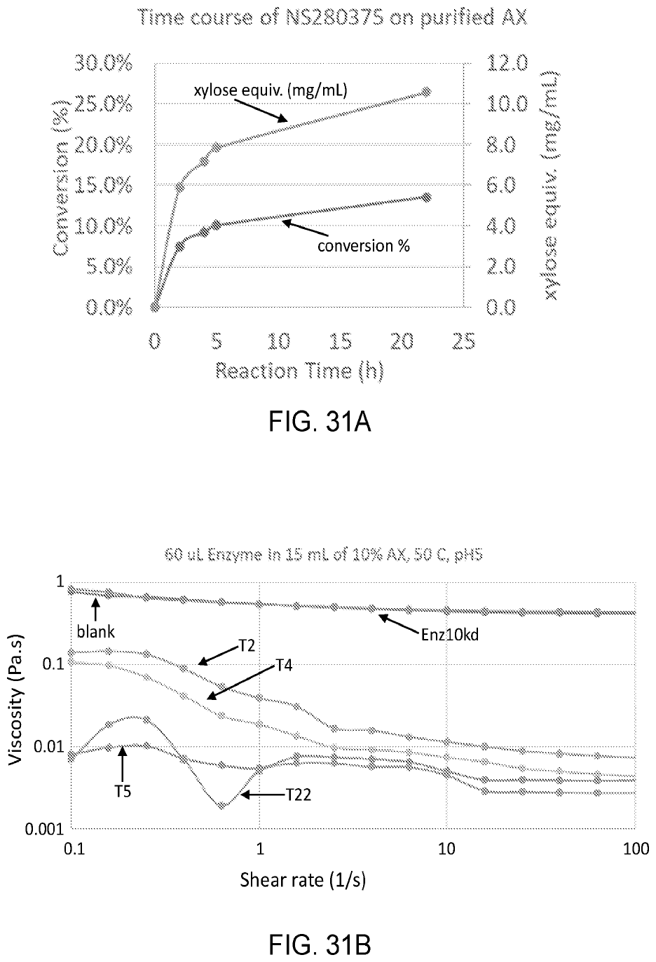

Each of the sample was analyzed for reducing end concentration (xylose equivalent and conversion, FIG. 31A), viscosity (FIG. 31B) and molecular weight. The max conversion achieved for this reaction was 13.6% at 22 h (Table 32).

The viscosity profile of AXOS solution with water and with the filtrate from enzyme sample overlapped, which indicates that the small molecule fraction does not impact the measured viscosity. The viscosity reached the lowest at 5 h, and there is no difference between the 5 h sample and the 22 h sample (FIG. 31B).

TABLE 32

| | | xylose | | xylose eqv. | |
|---|---|---|---|---|---|
| | | equivalent | Increase | Concentration | |
| Time (h) | A540 | (μg) | (μg) | (mg/mL) | Conversion % |
| 0 | 0.17 | 59.0 | 0.0 | 0.0 | 0.0% |
| 2 | 1.521 | 205.9 | 146.8 | 5.9 | 7.5% |
| 4 | 1.819 | 238.3 | 179.2 | 7.2 | 9.2% |
| 5 | 1.976 | 255.3 | 196.3 | 7.9 | 10.1% |
| 22 | 2.602 | 323.4 | 264.3 | 10.6 | 13.6% |

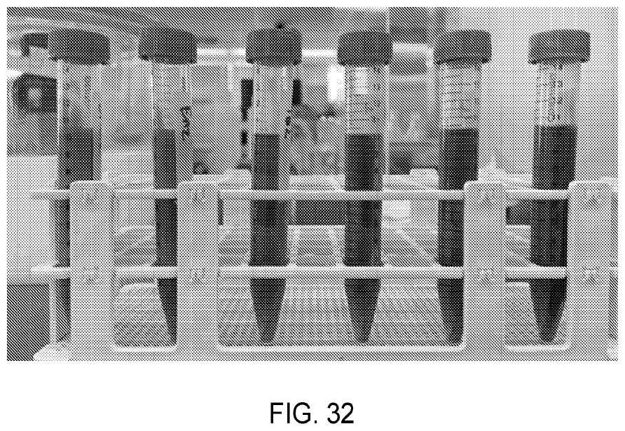

Fiber wash 2.0 treated AXOS can be clarified with centrifugation. After centrifugation at 6000 rpm for 10 min, all enzyme treated AXOS samples formed a clear supernatant, and a pellet at the bottom, while the non-treated samples did not (FIG. 32, from left to right: blank (no enzyme), with Enz10kD filtrate, with enzyme after 2 h reaction, with enzyme after 4 h reaction, with enzyme after 5 h reaction and with enzyme after 22 h reaction). The result indicated that lowering viscosity of the sample facilitated the precipitation of the insoluble. The Brix value of the supernatant obtained was between 9.5-10, very close to the initial solid content prepared (10%).

The supernatant from each enzyme treated sample was dried in the 70° C. oven overnight, and the protein content was measured. The blank and Enz10kD samples were dried as is. The protein content in each of the samples is listed in Table 33. Based on the results, protein content decreased in the AXOS sample from about 9.5-9.7% to 7.4-7.7% which suggests that the majority of protein is associated with the soluble AXOS. The molecular weight was also measured (Table 34, with DP1 included, Table 35, without DP1).

TABLE 33

| Description | % protein |
|---|---|
| blank | 9.785 |
| Enz10 kD | 9.5 |
| Dried supernatant after 2 h enzyme treatment | 7.471 |
| Dried supernatant after 4 h enzyme treatment | 7.632 |
| Dried supernatant after 5 h enzyme treatment | 7.701 |
| Dried supernatant after 22 h enzyme | 7.666 |
| treatment | |

TABLE 34

| Description | Mn | Mw | Polydispersity |
|---|---|---|---|
| Promitor 85 Control | 1208 | 2081 | 1.72 |
| Blank-AX no enzyme | 5447 | 161051 | 29.57 |
| AX with Enz10 kD | 5593 | 161752 | 28.97 |
| supernatant after 2 h enzyme treatment | 2001 | 31240 | 15.62 |
| supernatant after 4 h enzyme treatment | 1822 | 19803 | 10.87 |
| supernatant after 5 h enzyme treatment | 1829 | 20732 | 11.34 |
| supernatant after 22 h enzyme treatment | 1510 | 12714 | 8.42 |
| Example 16 | 2314 | 30246 | 13.08 |

TABLE 35

| Description | Mn | Mw | Polydispersity |
|---|---|---|---|
| Example 16 | 3754 | 31242 | 8.3 |
| supernatant after 22 h | 2834 | 13514 | 4.8 |
| enzyme treatment | | | |
| supernatant after 5 h | 3661 | 21911 | 6.0 |
| enzyme treatment | | | |
| supernatant after 4 h | 3664 | 20938 | 5.7 |
| enzyme treatment | | | |
| supernatant after 2 h | 4583 | 33095 | 7.2 |
| enzyme treatment | | | |
| AX with Enz10 kD | 10495 | 164080 | 15.7 |
| Blank-AX no enzyme | 10359 | 163476 | 15.8 |

### Enzyme C Reaction at Optimal Temperature

A volume of 20 mL of AX extract (DS %=4.2%, and carbohydrate content is 52%) adjusted to pH 5.0, and 5 mL was pipetted into two 15 mL centrifuge tubes. The solution was then placed in the thermomixer and heated at 70° C. and 300 rpm. A sample for baseline was collected by pipetting 50 μl of the AX solution into an Eppendorf tube with 750 μl DNSA reagent. 20 μl of Enzyme C was added to tube 1 and 40 μl Enzyme C was added to tube 2. At time zero (Immediately after enzyme addition), 1 h, 2 h, 3 h, 4 h and 5 h, an aliquot of 50 μl or 25 μl sample was transferred to Eppendorf tubes containing 750 μl DNSA reagent. Into each tube, 450 μl or 475 μl water was added to bring the volume to 1.25 mL. The samples were then heated at 98 C for 15 min, and 250 μl Rochelle's Salt was added subsequently in each tube. Absorption at 540 nm was recorded (Table 36).

TABLE 36

| | 20 μL Enzyme C | 40 μL Enzyme C |
|---|---|---|
| Time | Abs at 540 nm | Abs at 540 nm |
| Baseline | 0.139 | 0.136 |
| 0 | 0.143 | 0.142 |
| 1 | 0.154 | 0.157 |
| 2 | 0.161 | 0.163 |
| 3 | 0.128 | 0.129 |
| 4 | 0.132 | 0.126 |
| 5 | 0.132 | 0.125 |

No significant in the A540 increase at both enzyme concentrations, indicating that Enzyme C was not efficient in hydrolyzing AX under these conditions.

### Comparing FRONTIA® Fiber Wash and FRONTIA® Fiber wash 2.0 in the Hydrolysis of Corn Bran Arabinoxylan

Arabinoxylan products extracted as described herein were treated with an enzyme as follows:
1) Make 10% AX solution in DI water (5 g of Example 15 AX product dispersed in water to final weight of 50 g)
2) Adjust pH to 4.5;
3) Transfer 10 g of the solution to each of the four 15-mL centrifuge tube (save the rest of AX solution as time zero reference for later)
4) Add 10 μl FRONTIA® Fiber wash to each tube;
5) Place the tubes in the Thermomixer with the appropriate holder at 45° C. and 300 rpm;
6) At 1 h, 2 h, 4 h, 6 h, take 1 tube out to quench at 95 C for 10 min

The reaction product was analyzed on GPC for molecular weight, and the result was compared with that from the FRONTIA® Fiber wash 2.0 treatment (Table 34 above)

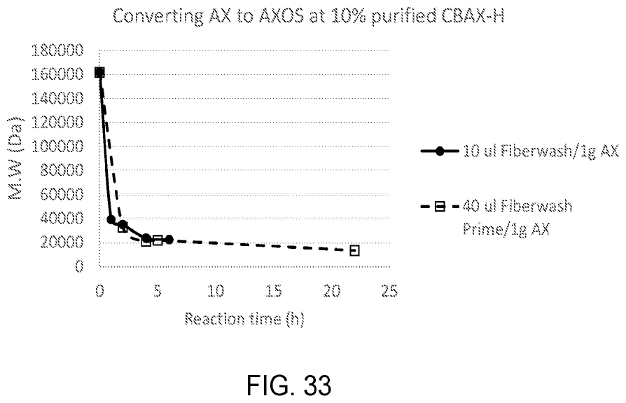

FRONTIA® Fiber wash was able to achieve similar molecular weight reduction at ¼ of the dosage needed for FRONTIA® Fiber wash 2.0. The time course of conversion of these two treated samples is reported in FIG. 33.

### Conclusions

The enzymes FRONTIA® Fiber wash and FRONTIA® Fiber wash 2.0 (Novozymes) were the most effective in hydrolyzing the corn bran arabinoxylan prepared. When the solution of corn bran AX is treated with these enzymes, viscosity of the solution was reduced and as a result, the insoluble contaminants was able to precipitate easily with centrifugation and improve the clarity of the solution. The other two enzymes (SHEARZYME® and Enzyme C) were not very effective in hydrolyzing corn bran AX.

### Example 18. Emulsifying Capability of Extracted Arabinoxylan

To evaluate the emulsification properties of the arabinoxylan extracted by the process as described herein, two emulsions were made. The first was prepared by adding 150 g of arabinoxylan extract (˜4.5% ds) at pH 7 to 50 g of oil and mixing (with IKA T25 ULTRATURAX Homogenizer) at 5000 rpm for 15 seconds and then homogenizing (with IKA T25 ULTRATURAX Homogenizer) at 11,000 rpm for 90 second. The second was prepared in the same manner but with the arabinoxylan extract being at a pH of 4. For comparison, an emulsion of Artesa 60, a chickpea protein, was also prepared. To make the comparison emulsion, 6 g of Artesa 60 was add to 144 g of DI water a mixed at 5000 rpm for 1 minute. To the solution, 50 g of oil was added and mixed as described above for the arabinoxylan emulsions.

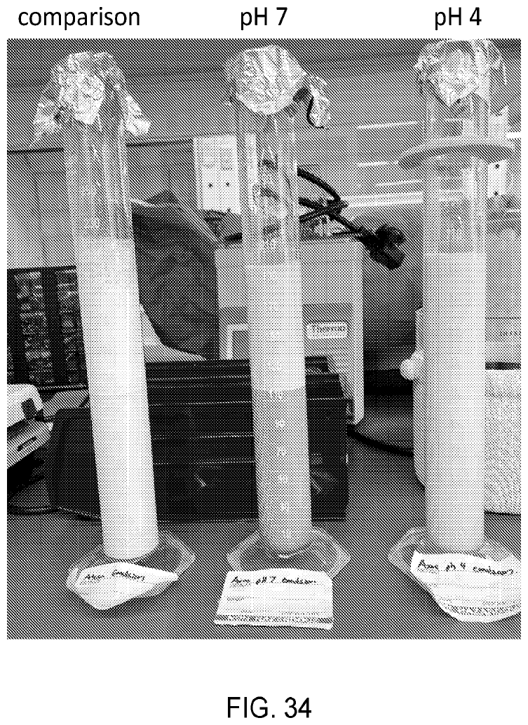

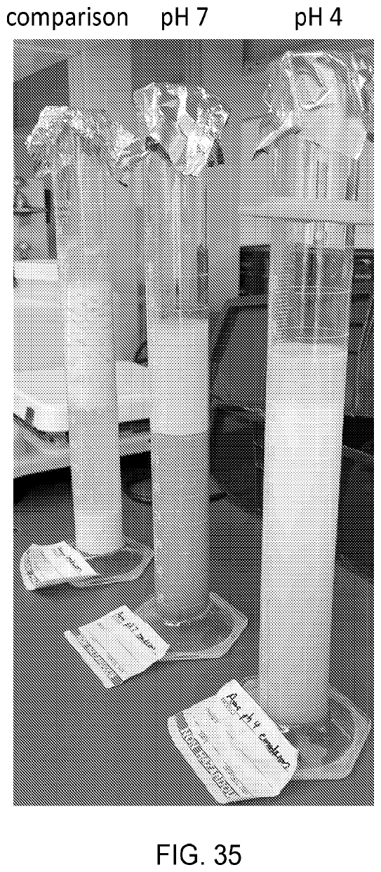

Each of these emulsions were transferred to a 100 mL graduated cylinder and the volume of cream layer and total volume was recorded. These volumes were recorded at 1 h, 2 h, 4 h, and 24 h, and the emulsion index was calculated for each reading. Table 37 reports these results. FIG. 34 is a picture of these three emulsions are 24 hours, and FIG. 35 is a picture after 6 days.

TABLE 37

| | Arabinoxylan Emulsion pH 7 | | | Arabinoxylan Emulsion pH 4 | | | Comparison Emulsion | | |
|---|---|---|---|---|---|---|---|---|---|
| | Cream | Total | Emulsion | Cream | Total | Emulsion | Cream | Total | Emulsion |
| Time | Volume | Volume | Index | Volume | Volume | Index | Volume | Volume | Index |
| 0 | 200 | 200 | 1 | 200 | 200 | 1 | 188 | 226 | 0.83 |
| 1 | 56 | 200 | 0.28 | 200 | 200 | 1 | 188 | 226 | 0.83 |
| 2 | 76 | 200 | 0.38 | 200 | 200 | 1 | 188 | 226 | 0.83 |
| 3 | 90 | 200 | 0.45 | 200 | 200 | 1 | 188 | 226 | 0.83 |
| 4 | 96 | 200 | 0.48 | 200 | 200 | 1 | 188 | 226 | 0.83 |
| 24 | 116 | 200 | 0.58 | 200 | 200 | 1 | 120 | 226 | 0.530 |

The arabinoxylan emulsion at pH 4 shows higher emulsion capacity than it is at pH 7.0, and much better stability. After 24 h, the emulsion at pH 4.0 does not show any phase separation, as shown in FIG. 34. In contrast, at pH 7.0, phase separation occurred within the first hour. The emulsion capability (emulsion index) of arabinoxylan emulsion at pH 4.0 is also better than that of the comparison emulsion.

Additional aspects of the disclosure are provided by the following non-limiting embodiments, which can be combined in any number and in any fashion not logically or technically inconsistent.

Embodiment 1. A method of providing an arabinoxylan product from corn fiber, the method comprising:
providing a corn material (e.g., a corn fiber);
de-starching the corn material to provide a de-starched com material (e.g., a de-starched corn fiber);
maintaining an aqueous mixture of the de-starched corn material at an extraction pH in the range of 5-14 for a time and at a temperature effective to extract arabinoxylan from the de-starched corn material into an aqueous phase of the aqueous mixture and to provide a corn residue (e.g., a corn fiber residue);
separating the aqueous phase containing the extracted arabinoxylan from the corn residue; and
providing the arabinoxylan product from the aqueous phase.

Embodiment 2. The method of embodiment 1, wherein the corn material is corn fiber, e.g., as a by-product of com wet-milling.

Embodiment 3. The method of embodiment 2, wherein the corn material is corn pericarp.

Embodiment 4. The method of any of embodiments 1-3, wherein the corn material is corn fiber provided as an aqueous mixture having a degree of solids in the range of 5-40% (e.g., in the range of 5-35%, or 5-30%, or 10-40%, or 10-35%, or 10-30%, or 15-40%, or 15-35%. or 15-30%, or 20-40%, or 20-35%, or 20-30%, or 25-40%, or 25-35%, or 25-30%).

Embodiment 5. The method of any of embodiments 1-4, wherein de-starching the com material comprises washing the com material with a washing aqueous medium (e.g., water) having a temperature up to 80° C., e.g., up to 75° C. or up to 70° C.

Embodiment 6. The method of embodiment 5, wherein the washing aqueous medium has a temperature in the range of 20-80° C., e.g., or 20-75° C., or 20-70° C., or 30-80° C., or 30-75° C., or 30-70° C., or 40-80° C., or 40-75° C., or 40-70° C., or 50-80° C., or 50-75° C. or 50-70° C.

Embodiment 7. The method of embodiments 5 or 6, wherein the mass of the washing aqueous medium is at least 5 times the mass on a dry solids basis of the corn material, e.g., at least 10 times or at least 20 times.

Embodiment 8. The method of any of embodiments 5-7, wherein the washing is performed for a time that is no more than an hour, e.g., no more than 45 minutes, or no more than 30 minutes.

Embodiment 9. The method of any of embodiments 1-8, wherein de-starching the com material comprises treating the corn material with one or more enzymes in an enzyme treatment aqueous medium.

Embodiment 10. The method of embodiment 9, wherein the one or more enzymes includes one or more amylases, one or more glucoamylases, one or more xylanases, one or more arabinofuranosidases, and/or one or more cellulases.

Embodiment 11. The method of embodiments 9 or 10, wherein treating the com material with one or more enzymes in an enzyme treatment aqueous medium is conducted for a time of at least 30 minutes (e.g., at least 1 hour, at least 1.5 hours, at least 2 hours, at least 2.5 hours, at least 3 hours, or at least 3.5 hours).

Embodiment 12. The method of any of embodiments 9-11, wherein treating the corn material with one or more enzymes in an enzyme treatment aqueous medium is conducted for a temperature in the range of 25° C. to 70° C. (e.g., in the range of 25° C. to 60° C., or 30° C. to 70° C., or 30° C. to 60° C.).

Embodiment 13. The method of any of embodiments 9-12, wherein treating the corn material with one or more enzymes in an enzyme treatment aqueous medium is conducted at a pH in the range of 3 to 9. (e.g. in the range of 3 to 8, or 3 to 7, or 4 to 9, or 4 to 8, or 4 to 7).

Embodiment 14. The method of any of embodiments 9-13, wherein after treating the corn material with one or more enzyme treatment aqueous medium, the de-starched corn material is washed with an aqueous material.

Embodiment 15. The method of embodiment 14, wherein the mass of the washing aqueous medium is in the range of 75%-200% of the mass of the corn material.

Embodiment 16. The method of embodiment 14, wherein the mass of the washing aqueous medium is at least 2 times, e.g., at least 3, the mass of the com material.

Embodiment 17. The method of any of embodiments 1-16, wherein the de-starching reduces a starch content of the corn material by at least 1 wt. % (e.g., at least 5 wt. %, 10 wt. %, or 15 wt. %).

Embodiment 18. The method of any of embodiments 1-17, wherein the de-starched corn material has a starch content of no more than 20 wt. % (e.g., no more than 15 wt. %, or no more than 12 wt. %, or no more than 10 wt. %, or no more than 8 wt. %, or no more than 5 wt. %) on a dry solids basis.

Embodiment 19. The method of any of embodiments 1-18, wherein the extraction pH is in the range 5 to 13, or 5 to 12.5, or 5 to 12, or 5 to 11.7, or 5 to 11, or 5 to 10, or 5 to 9, or 6 to 14, or 6 to 13, or 6 to 12.5, or 6 to 12, or 6 to 11.7, or 6 to 11, or 6 to 10, or 7 to 14, or 7 to 13, or 7 to 12.5, or 7 to 12, or 7 to 11.7, or 7 to 11, or 7 to 10, or 8 to 14, or 8 to 13, or 8 to 12.5, or 8 to 12, or 8 to 11.7, or 8 to 11, or 8 to 10.

Embodiment 20. The method of any of embodiments 1-18, wherein the extraction pH is in the range of 6.2 to 14, e.g., in the range of 6.2 to 13, or 6.2 to 12, or 6.2 to 11.7, or 6.2 to 11, or 6.2 to 10, or 6.2 to 9, or 7.2 to 14, or 7.2 to 13, or 7.2 to 12.5, or 7.2 to 12, or 7.2 to 11.7, or 7.2 to 11, or 7.2 to 10, or 8.2 to 14, or 8.2 to 13, or 8.2 to 12.5, or 8.2 to 12, or 8.2 to 11.7, or 8.2 to 11, or 8.2 to 10.

Embodiment 21. The method of any of embodiments 1-18, wherein the extraction pH is in the range of 6.5 to 14, e.g., in the range of 6.5 to 13, or 6.5 to 12, 6.5 to 11.7, or 6.5 to 11, or 6.5 to 10, or 6.5 to 9, or 7.5 to 14, or 7.5 to 13, or 7.5 to 12.5, or 7.5 to 12, or 7.5 to 11.7, or 7.5 to 11, or 7.5 to 10, or 8.5 to 14, or 8.5 to 13, or 8.5 to 12.5, or 8.5 to 12, or 8.5 to 11.7, or 8.5 to 11, or 8.5 to 10.

Embodiment 22. The method of any of embodiments 1-18, wherein the extraction pH is in the range of 9 to 14, e.g., in the range of 9 to 13, or 9 to 12.5, or 9 to 12, or 9 to 11.7, or 9 to 11, or 10 to 14, or 10 to 13, or 10 to 12.5, or 10 to 12, or 10 to 11.7, or 10.5 to 14, or 10.5 to 13, or 10.5 to 12.5, or 10.5 to 12, or 11 to 14, or 11 to 13, or 11 to 12.5, or 11.7 to 14, or 11.7 to 13, or 11.7 to 12.5.

Embodiment 23. The method of any of embodiments 1-18, wherein the extraction pH is in the range of 5 to 9, e.g., in the range of 5 to 8.5, or 5 to 8.2, or 5 to 8, or 5 to 7.5, or 5 to 7.2, or 5 to 7.

Embodiment 24. The method of any of embodiments 1-18, wherein the extraction pH is in the range of 6 to 9, e.g., in the range of 6 to 8.5, or 6 to 8.2, or 6 to 8, or 6 to 7.5, or 6 to 7.2, or 6 to 7.

Embodiment 25. The method of any of embodiments 1-18, wherein the extraction pH is in the range of 6.2 to 9, e.g., in the range of 6.2 to 8.5, or 6.2 to 8.2, or 6.2 to 8, or 6.2 to 7.5, or 6.2 to 7.2, or 6.2 to 7.

Embodiment 26. The method of any of embodiments 1-18, wherein the extraction pH is in the range of 6.5 to 9, e.g., in the range of 6.5 to 8.5, or 6.5 to 8.2, or 6.5 to 8, or 6.5 to 7.5, or 6.5 to 7.2, or 6.5 to 7.

Embodiment 27. The method of any of embodiments 1-18, wherein the extraction pH is in the range of 10-14, for example, 10-13.7, or 10-13.5, or 10-13.3, or 10-13, or 10-12.7, or 10-12.5, or 10-12.3, or 10-12, or 10-11.7, or 10-11.5, or 10-11.3, or 10-11.

Embodiment 28. The method of any of embodiments 1-18, wherein the extraction pH is in the range of 10.3-14, for example, 10.3-13.7, or 10.3-13.5, or 10.3-13.3, or 10.3-13, or 10.3-12.7, or 10.3-12.5, or 10.3-12.3, or 10.3-12, or 10.3-11.7, or 10.3-11.5, or 10.3-11.3, or 10.3-11.

Embodiment 29. The method of any of embodiments 1-18, wherein the extraction pH is in the range of 11.5-14, for example, 10.5-13.7, or 10.5-13.5, or 10.5-13.3, or 10.5-13, or 10.5-12.7, or 10.5-12.5, or 10.5-12.3, or 10.5-12, or 10.5-11.7, or 10.5-11.5, or 10.5-11.3, or 10.5-11.

Embodiment 30. The method of any of embodiments 1-18, wherein the extraction pH is in the range of 10.7-14, for example, 10.7-13.7, or 10.7-13.5, or 10.7-13.3, or 10.7-13, or 10.7-12.7, or 10.7-12.5, or 10.7-12.3, or 10.7-12, or 10.7-11.7, or 10.7-11.5, or 10.7-11.3.

Embodiment 31. The method of any of embodiments 1-18, wherein the extraction pH is in the range of 11-14, for example, 11-13.7, or 11-13.5, or 11-13.3, or 11-13, or 11-12.7, or 11-12.5, or 11-12.3, or 11-12, or 11-11.7, or 11-11.5.

Embodiment 32. The method of any of embodiments 1-18, wherein the extraction pH is in the range of 11.3-14, for example, 11.3-13.7, or 11.3-13.5, or 11.3-13.3.

Embodiment 33. The method of any of embodiments 1-18, wherein the extraction pH is in the range of 11.3-13, for example, 11.3-12.7, or 11.3-12.5, or 11.3-12.3, or 11.3-12, or 11.3-11.7.

Embodiment 34. The method of any of embodiments 1-18, wherein the extraction pH is in the range of 11.5-14, for example, 11.5-13.7, or 10-13.5, or 11.5-13.3, or 11.5-13, or 11.5-12.7, or 11.5-12.5, or 11.5-12.3, or 11.5-12.

Embodiment 35. The method of any of embodiments 1-18, wherein the extraction pH is in the range of 11.7-14, for example, 11.7-13.7, or 10-13.5, or 11.7-13.3, or 11.7-13, or 11.7-12.7, or 11.7-12.5, or 11.7-12.3.

Embodiment 36. The method of any of embodiments 1-25, wherein one or more pH adjustments are performed on the aqueous mixture of de-starched corn material to provide the aqueous mixture with the extraction pH.

Embodiment 37. The method of embodiment 36, wherein the one or more pH adjustments are performed by the addition of an alkaline solution.

Embodiment 38. The method of embodiment 36, wherein the one or more pH adjustments are performed by the addition of an alkaline solid (e.g., sodium hydroxide, calcium hydroxide, or potassium hydroxide).

Embodiment 39. The method of embodiment 36, wherein the one or more pH adjustments are performed by addition of water having a pH in the range of 5-9.

Embodiment 40. The method of any of embodiments 1-39, wherein the maintaining step is conducted for a time of at least 1 hour (e.g., at least 2 hours, or at least 3 hours).

Embodiment 41. The method of any of embodiments 1-39, wherein the maintaining step is conducted for a time in the range of 1 to 6 hours (e.g., in the range of 1 to 4 hours, or 2 to 6 hours, or 2 to 4 hours, or 3 to 6 hours).

Embodiment 42. The method of any of embodiments 1-41, wherein the maintaining step is conducted at a temperature in the range of 70° C. to 150° C. (e.g., in the range of 70° C. to 140° C., or 70° C. to 130° C., 70° C. to 120° C., or 70° C. to 110° C., or 70° C. to 100° C., or 80° C. to 150° C., or 80° C. to 140° C., or 80° C. to 130° C., or 80° C. to 120° C., or 80° C. to 110° C., or 80° C. to 100° C., or 90° C. to 150° C., or 90° C. to 140° C., or 90° C. to 130° C., to 90° C. to 120° C., or 90° C. to 110° C., or 90° C. to 100° C.).

Embodiment 43. The method of any of embodiments 1-42, further comprising adjusting the pH of the aqueous mixture to a pH in the range of 7 to 10, e.g., in the range of 7 to 9.5, or 7 to 9.2, or 7 to 9, after the maintaining step.

Embodiment 44. The method of any of embodiments 1-43, wherein the separating step is accomplished by filtration, centrifugation, or a combination thereof.

Embodiment 45. The method of any of embodiments 1-44, wherein no further cleaning steps are performed after the separating step.

Embodiment 46. The method of any of embodiments 1-44, wherein after the separating step, the extracted arabinoxylan is filtered (e.g., via nanofiltration and/or diafiltration).

Embodiment 47. The method of embodiment 46, wherein the extracted arabinoxylan is filtered with a filter having a molecular weight cut-off of at least 0.5 kDa (e.g., at least 1 kDa).

Embodiment 48. The method of any of embodiments 1-47, wherein providing the arabinoxylan product from the aqueous phase comprises concentrating the aqueous phase containing the extracted arabinoxylan oligosaccharide.

Embodiment 49. The method of any of embodiments 1-48, wherein providing the arabinoxylan product from the aqueous phase comprises spray-drying the aqueous phase containing the extracted arabinoxylan oligosaccharide.

Embodiment 50. The method of any of embodiments 1-49, wherein the arabinoxylan product is provided as a solid.

Embodiment 51. The method of embodiment 50, wherein the arabinoxylan product has a total dry solids content in an amount of at least 90% by weight (e.g., at least 95 wt %).

Embodiment 52. The method of any of embodiments 1-51, wherein the arabinoxylan product is provided as a syrup.

Embodiment 53. The method of embodiment 52, wherein the arabinoxylan product has a total dry solids content in an amount of at least 50% (e.g., at least 60%, at least 70%, or at least 80%) by weight.

Embodiment 54. The method of embodiment 53, wherein the arabinoxylan product has a total dry solids content in an amount in the range of 50-90% (e.g., in the range of 50-85%, or 50-80%, or 50-75%, or 50-80%, or 55-90%, or 55-85%, or 55-80%, or 55-75%, or 60-90%, or 60-85%, or 60-80%, or 60-75%, or 65-90%, or 65-85%, or 65-80%, or 65-75%) by weight.

Embodiment 55. The method of any of embodiments 1-54, wherein the arabinoxylan product has a number average molecular weight in the range of 2000 to 6000 g/mol (e.g., in the range of 3000 to 6000 g/mol, or 4000 to 6000 g/mol, or 2000 to 5000 g/mol, or 3000 to 5000 g/mol).

Embodiment 56. The method of any of embodiments 1-55, wherein the extracted arabinoxylan oligosaccharides have a weight average molecular weight in the range of 50,0000 to 400,000 g/mol (e.g., in the range of 100,000 to 400,000 g/mol, or 150,000 to 400,000 g/mol, or 200,000 to 400,000 g/mol, or 50,000 to 300,000 g/mol, or 100,000 to 300,000 g/mol, or 150,000 to 300,000 g/mol, or 200.000 to 300,000 g/mol, or 50,000 to 200,000 g/mol, or 100,000 or 200,000 g/mol, or 150,000 or 200,000 g/mol).

Embodiment 57. The method of any of embodiments 1-56, wherein the arabinoxylan product has a polydispersity in the range of 5-25 (e.g., in the range of 8-25, or 10-25, or 12-25, or 15-25, or 5-20, or 8-20, or 10-20, or 15-20).

Embodiment 58. The method of any of embodiments 1-57, further comprising hydrolyzing the arabinoxylan product to reduce its weight average molecular weight.

Embodiment 59. The method of any of embodiments 1-57, further comprising condensing the arabinoxylan product to increase its weight average molecular weight.

Embodiment 60. The method of any of embodiments 1-59, further comprising treating the arabinoxylan product to decrease its viscosity.

Embodiment 61. The method of embodiment 60, wherein treating the arabinoxylan product comprises contacting the arabinoxylan product with one or more enzymes in a second enzyme treatment aqueous medium.

Embodiment 62. The method of embodiment 61, wherein the one or more enzymes includes one or more amylases, one or more glucoamylases, one or more xylanases, one or more arabinofuranosidases, and/or one or more cellulases.

Embodiment 63. The method of embodiment 61, wherein the one or more enzymes includes one or more endo-xylanases.

Embodiment 64. The method of embodiments 61-63, wherein treating the arabinoxylan product comprises contacting the arabinoxylan product with one or more enzymes in a second enzyme treatment aqueous medium is conducted for a time of at least 15 minutes (e.g., at least 30 minutes, at least 45 minutes, or at least 60 minutes).

Embodiment 65. The method of embodiments 61-63, wherein treating the arabinoxylan product comprises contacting the arabinoxylan product with one or more enzymes in a second enzyme treatment aqueous medium is conducted for a time of at least 2 hours (e.g., at least 3 hours, at least 4 hours, or at least 5 hours).

Embodiment 66. The method of any of embodiments 61-65, wherein treating the arabinoxylan product comprises contacting the arabinoxylan product with one or more enzymes in a second enzyme treatment aqueous medium is conducted for a temperature in the range of 25° C. to 70° C. (e.g., in the range of 25° C. to 60° C., or 30° C. to 70° C., or 30° C. to 60° C.).

Embodiment 67. The method of any of embodiments 61-66, wherein treating the arabinoxylan product comprises contacting the arabinoxylan product with one or more enzymes in a second enzyme treatment aqueous medium is conducted at a pH in the range of 3 to 9, (e.g. in the range of 3 to 8, or 3 to 7, or 4 to 9, or 4 to 8, or 4 to 7).

Embodiment 68. The method of any of embodiments 1-67, wherein the arabinoxylan product comprises:
a dietary fiber content in the range of 60-95% by weight on a dry solids basis;
a digestible carbohydrate content of less than 20% by weight on a dry solids basis;
a protein content of less than 15% by weight on a dry solids basis; and
a fat content of less than 10% by weight on a dry solids basis.

Embodiment 69. An arabinoxylan product prepared by the method of any of embodiments 1-68.

Embodiment 70. The arabinoxylan product of embodiment 69, wherein the arabinoxylan product has:
a dietary fiber content in the range of 60-95% by weight on a dry solids basis;
a digestible carbohydrate content of less than 20% by weight on a dry solids basis;
a protein content of less than 15% by weight on a dry solids basis; and
a fat content of less than 10% by weight on a dry solids basis.

Embodiment 71. The method or arabinoxylan product of any of embodiments 1-70, wherein the dietary fiber content is in the range of 60-90% (e.g., in the range of 60-85%, or 60-80%, or 65-85%, or 65-80%, or 70-85%, or 70-80%, or 75-90%, or 75-85%, or 75-80%, or 80-90%, or 80-85%) by weight on a dry solids basis.

Embodiment 72. The method or arabinoxylan product of any of embodiments 1-71, wherein the digestible carbohydrate content is less than 15% (e.g., less than 12%, or 10%) by weight on a dry solids basis.

Embodiment 73. The method or arabinoxylan product of any of embodiments 1-72, wherein a ratio of dietary fiber to digestible carbohydrate is at least 4:1 (e.g., at least 5:1, or 6:1, or 7:1, or 8:1) by weight on a dry solids basis.

Embodiment 74. The method or arabinoxylan product of any of embodiments 1-73, wherein a ratio of dietary fiber to digestible carbohydrate is in the range of 4-20:1 (e.g., in the range of 5-20:1, or 7-20:1, or 4-10:1, or 5-10:1, or 7-10:1).

Embodiment 75. The method or arabinoxylan product of any of embodiments 1-74, wherein the protein content is less than 12% (e.g., less than 8% or less than 5%) by weight on a dry solids basis.

Embodiment 76. The method or arabinoxylan product of any of embodiments 1-74, wherein the protein content is in the range of 1-15% (e.g., in the range of 1-12%, or 1-10%, or 1-8%, or 1-6%, or 1-4%, or 2-8% or 2-6%, or 2-4%) by weight on a dry solids basis.

Embodiment 77. The method or arabinoxylan product of any of embodiments 1-76, wherein the fat content is less than 8% (e.g., less than 5%, or 3%, or 2%) by weight on a dry solids basis.

Embodiment 78. The method or arabinoxylan product of any of embodiments 1-77, wherein the arabinoxylan product comprises an ash content of less than 10% by weight.

Embodiment 79. The method or arabinoxylan product of any of embodiments 1-78, wherein the arabinoxylan product comprises arabinose, xylose, galactose, and glucose.

Embodiment 80. The method or arabinoxylan product of any of embodiments 1-79, wherein the arabinoxylan product comprises arabinose in an amount of no more than 50% by weight (e.g., no more than 45% by weight, or no more than 40% by weight) on a dry solids basis.

Embodiment 81. The method or arabinoxylan product of any of embodiments 1-80, wherein the arabinoxylan product comprises xylose in an amount of no more than 50% by weight (e.g., no more than 45% by weight, or no more than 40% by weight) on a dry solids basis.

Embodiment 82. The method or arabinoxylan product of any of embodiments 1-81, wherein the arabinoxylan product comprises galactose in an amount of less than 5% by weight (e.g., less than 4.5% by weight, less than 4% by weight, or less than 3.5% by weight, or less than 3% by weight) on a dry solids basis.

Embodiment 83. The method or arabinoxylan product of any of embodiments 1-82, wherein the arabinoxylan product comprises glucose in an amount of less than 5% by weight (e.g., less than 4% by weight, or less than 3% by weight, or less than 2% by weight) on a dry solids basis.

Embodiment 84. The method or arabinoxylan product of any of embodiments 1-83, wherein the arabinoxylan product comprises xylose and arabinose in a weight ratio of no more than 1:5:1 (e.g., no more than 1.4:1, or no more than 1:3:1, or no more than 1.2:1, or no more than 1.1:1) on dry solids basis.

Embodiment 85. The method or arabinoxylan product of any of embodiments 1-84, wherein the arabinoxylan product comprises ferulic acid in an amount of at least 500 μg/g of arabinoxylan product.

Embodiment 86. The method or arabinoxylan product of any of embodiments 1-85, wherein the arabinoxylan product comprises ferulic acid in an amount of no more 15,000 μg/g of arabinoxylan product.

Embodiment 87. The arabinoxylan product of any of embodiments 69-86, wherein the arabinoxylan product is provided as a solid.

Embodiment 88. The arabinoxylan product of embodiment 87, wherein the arabinoxylan product has a total dry solids content in an amount of at least 90% by weight (e.g., at least 95 wt %).

Embodiment 89. The arabinoxylan product of any of embodiments 69-86, wherein the arabinoxylan product is provided as a syrup.

Embodiment 90. The arabinoxylan product of embodiment 89, wherein the arabinoxylan product has a total dry solids content in an amount of at least 50% (e.g., at least 60%, at least 70%, or at least 80%) by weight.

Embodiment 91. The arabinoxylan product of embodiment 89, wherein the arabinoxylan product has a total dry solids content in an amount in the range of 50-90% (e.g., in the range of 50-85%, or 50-80%, or 50-75%, or 50-80%, or 55-90%, or 55-85%, or 55-80%, or 55-75%, or 60-90%, or 60-85%, or 60-80%, or 60-75%, or 65-90%, or 65-85%, or 65-80%, or 65-75%) by weight.

Embodiment 92. The arabinoxylan product of any of embodiments 69-86, wherein the arabinoxylan product is provided as an emulsion (e.g., an oil-in-water emulsion).

Embodiment 93. The arabinoxylan product of embodiment 92, wherein the emulsion comprises an aqueous phase comprising the arabinoxylan product and oil in a weight ratio of at least 1:1 (e.g., at least 2:1, or at least 3:1).

Embodiment 94. The arabinoxylan product of embodiment 93, wherein the aqueous phase comprising the arabinoxylan product has a pH of at least 3 (e.g., at least 3.2, at least 3.5, or at least 4).

Embodiment 95. The arabinoxylan product of embodiment 93, wherein the aqueous phase comprising the arabinoxylan product has a pH in the range of 3-8 (e.g., in the range of 3-7, or 3-6, or 4-8, or 4-7, or 4-6).

Embodiment 96. The arabinoxylan product of any of embodiments 93-95, wherein the aqueous phase comprises the arabinoxylan product in an amount of at least 3% degree of solids (e.g., at least 3.5% degree of solids, at least 4.0% degree of solids, or at least 4.5% degree of solids).

Embodiment 97. The arabinoxylan product of any of embodiments 93-96, wherein the aqueous phase comprises the arabinoxylan product in an amount of no more than 10% degree of solids (e.g., no more than 8% degree of solids, or no more than 5% degree of solids).

Embodiment 98. The arabinoxylan product of any of embodiments 69-97, wherein the arabinoxylan product has a number average molecular weight in the range of 2000 to 6000 g/mol (e.g., in the range of 3000 to 6000 g/mol, or 4000 to 6000 g/mol, or 2000 to 5000 g/mol, or 3000 to 5000 g/mol).

Embodiment 99. The arabinoxylan product of any of embodiments 69-97, wherein the extracted arabinoxylan oligosaccharides have a weight average molecular weight in the range of 50,0000 to 400,000 g/mol (e.g., in the range of 100,000 to 400,000 g/mol, or 150,000 to 400,000 g/mol, or 200,000 to 400,000 g/mol, or 50,000 to 300,000 g/mol, or 100,000 to 300,000 g/mol, or 150,000 to 300,000 g/mol, or 200,000 to 300,000 g/mol, or 50,000 to 200,000 g/mol, or 100,000 or 200,000 g/mol, or 150,000 or 200,000 g/mol).

Embodiment 100. The arabinoxylan product of any of embodiments 69-99, wherein the arabinoxylan product has a polydispersity in the range of 5-25 (e.g., in the range of 8-25, or 10-25, or 12-25, or 15-25, or 5-20, or 8-20, or 10-20, or 15-20).

Embodiment 101. A method for making a food product, the method comprising:
providing an arabinoxylan product according to any of embodiments 69-100 or made by a method according to any of embodiments 1-68, and
combining the arabinoxylan product with one or more other food ingredients.

Embodiment 102. A food product made by a method according to embodiment 101.

Embodiment 103. A food product comprising an arabinoxylan product according to any of embodiments 69-100 or made by a method according to any of embodiments 1-68.

Embodiment 104. The food product of embodiment 103, made by a method according to embodiment 95.

Embodiment 105. The method or food product according to any of embodiments 101-104, wherein the food product comprises a confectionary composition, e.g., a chocolate composition, in which the arabinoxylan product is disposed.

Embodiment 106. The method or food product according to embodiment 105, wherein the food product is a candy, a bar (e.g., energy bar, snack bar, breakfast bar), a frozen dessert or a baked good comprising the confectionary composition (e.g., chocolate composition, confectionary coating composition, enrobed baked good, or baked good with inclusions of chocolate or confectionary coating composition).

Embodiment 107. The method or food product according to any of embodiments 101-104, wherein the food product is a fatty spread.

Embodiment 108. The method or food product according to any of embodiments 101-104, wherein the food product is a cereal, a granola, a muesli, a topping, a coating, a baked good (e.g., cookie, a biscuit, a bread, a pastry, a pizza crust, a flatbread), a bar (e.g., snack bar, cereal bar, granola bar, energy bar), a meat alternative, a filling (e.g., a fruit filling or a crème filling), a fruit snack such as a fruit leather, a pasta, a sweetener, a frozen dessert, a dairy product (e.g., a yogurt, a quark, an ice cream), a dairy alternative product (e.g. yoghurt alternative), a glaze, a frosting, a beverage, a syrup, a pet food, a medical food, a flavoring, or a dry blend.

Embodiment 109. The method or food product according to any of embodiments 101-104, wherein the food product is a meat alternative or meat substitute.

Embodiment 110. The method or food product according to any of embodiments 101-104, wherein the food product is a beverage (e.g., a dairy drink, tea, coffee, water, slimming beverages, coarse grain drinks, fermented beverages, or malted beverages).

1-30. (canceled)

31. A method of providing an arabinoxylan product from corn fiber, the method comprising:
providing a corn material;
de-starching the corn material to provide a de-starched corn material;
maintaining an aqueous mixture of the de-starched corn material at an extraction pH in the range of 5-14 for a time and at a temperature effective to extract arabinoxylan from the de-starched corn material into an aqueous phase of the aqueous mixture and to provide a corn residue;
separating the aqueous phase containing the extracted arabinoxylan from the corn residue; and
providing the arabinoxylan product from the aqueous phase.

32. The method of claim 31, wherein the corn material is corn fiber or is corn pericarp.

33. The method of claim 31, wherein de-starching the corn material comprises treating the corn material with one or more enzymes in an enzyme treatment aqueous medium, wherein the one or more enzymes includes one or more amylases, one or more glucoamylases, one or more xylanases, one or more arabinofuranosidases, and/or one or more cellulases.

34. The method of claim 31, wherein the de-starching reduces a starch content of the corn material by at least 10 wt. %.

35. The method of claim 31, wherein the extraction pH is in the range of 5 to 9.

36. The method of claim 31, wherein the extraction pH is in the range of 9 to 14.

37. The method of claim 31, wherein the maintaining step is conducted for a time of at least 1 hour and at a temperature in the range of 70° C. to 150° C.

38. The method of claim 31, wherein the separating step is accomplished by filtration, centrifugation, or a combination thereof.

39. The method of claim 31, wherein after the separating step, the extracted arabinoxylan is filtered.

40. The method of claim 31, wherein the arabinoxylan product has a number average molecular weight in the range of 2000 to 6000 g/mol.

41. The method of claim 31, wherein the extracted arabinoxylan oligosaccharides have a weight average molecular weight in the range of 50,000 to 400,000 g/mol.

42. The method of claim 31, wherein the arabinoxylan product has a polydispersity in the range of 5-25.

43. The method of claim 31, further comprising hydrolyzing the arabinoxylan product to reduce its weight average molecular weight.

44. The method of claim 31, further comprising treating the arabinoxylan product to decrease its viscosity by contacting the arabinoxylan product with one or more enzymes in a second enzyme treatment aqueous medium, the one or more enzymes including one or more amylases, one or more glucoamylases, one or more xylanases, one or more arabinofuranosidases, and/or one or more cellulases.

45. The method of claim 31, further comprising treating the arabinoxylan product to decrease its viscosity by contacting the arabinoxylan product with one or more enzymes in a second enzyme treatment aqueous medium, the one or more enzymes including one or more endo-xylanases.

46. The method of claim 31, wherein the arabinoxylan product comprises:
a dietary fiber content in the range of 60-95% by weight on a dry solids basis;
a digestible carbohydrate content of less than 20% by weight on a dry solids basis;
a protein content of less than 15% by weight on a dry solids basis; and
a fat content of less than 10% by weight on a dry solids basis.

47. An arabinoxylan product prepared by the method of claim 31.

48. An arabinoxylan product comprising:
a dietary fiber content in the range of 60-95% by weight on a dry solids basis;
a digestible carbohydrate content of less than 20% by weight on a dry solids basis;
a protein content of less than 15% by weight on a dry solids basis; and
a fat content of less than 10% by weight on a dry solids basis.

49. A method for making a food product, the method comprising:
providing an arabinoxylan product made by a method according to claim 31, and
combining the arabinoxylan product with one or more other food ingredients.

50. A food product comprising an arabinoxylan product made by a method according to claim 31.
